(19) United States
(12) Patent Application Publication
(10) Pub. No.: US 2013/0115322 A1
(43) Pub. Date: May 9, 2013

(54) Therapeutic Compositions and Methods for Treating Cell Dysplasia Using Extracts From Raspberry and Strawberry

(71) Applicants: Gary D. Stoner, Columbus, OH (US); John M. Cassady, Columbus, OH (US)

(72) Inventors: Gary D. Stoner, Columbus, OH (US); John M. Cassady, Columbus, OH (US)

(21) Appl. No.: 12/137,952

(22) Filed: Jun. 12, 2008

Related U.S. Application Data
(63) Continuation-in-part of application No. 10/951,413, filed on Sep. 28, 2004, now abandoned.

(30) Foreign Application Priority Data
Feb. 28, 2003 (US) PCT/US03/06279

(51) Int. Cl. A61K 36/73

(52) U.S. Cl. 424/765

(57) ABSTRACT

Isolated fruit extracts and components along with a method for treating or preventing a disease or condition by providing a fruit extract fraction having a therapeutically effective amount of activity in modulating undesired signal transduction activity useful for reducing the frequency, duration or severity of a disease or condition in a subject. The fruit extracts are preferably derived from a plant of one or more of the genera Fragaria or Rubus. The isolated fruit extracts or compositions provide anti-dysplastic activity and modulate signal transduction activity by inhibiting one or more of AP-1, NFkB, Akt, COX-2, and VEGF.

### CROSS-REFERENCE TO RELATED APPLICATIONS

This application is a continuation-in-part of U.S. patent application Ser. No. 10/951,413, which is an application under 35 U.S.C. §371 of PCT application No. PCT/US03/06279, filed Feb. 28, 2003 which claims priority to U.S. Provisional Application No. 60/360,783 filed on Mar. 1, 2002, U.S. Provisional Application No. 60/369,160 filed on Mar. 29, 2002, and U.S. Provisional Application No. 60/425,829 filed on Nov. 12, 2002, and the contents of all of the foregoing are incorporated herein by reference.

### STATEMENT REGARDING FEDERALLY SPONSORED RESEARCH

This work was supported, in part, by grants from the National Cancer Institute (RO1CA103180; RO1 CA96130), National Institute of Dental and Craniofacial Research (PO1 DE12704), and U.S. Department of Agriculture grants 2003-34501-13965, 2005-38903-02313 and 2006-38903-3560.

### BACKGROUND OF THE INVENTION

For thousands of years, humankind has relied on plant derivatives for the prevention and treatment of a wide variety of ailments. For example, in China, various teas have been used as a crude medicine for over 4,000 years. And more recently, there has been considerable interest in taking advantage of various plant extracts as a source of health promoting substances such as natural oxidants, phenolic compounds, flavonoids, tocochromanols, and beneficial fatty acids. In part, this trend is due to a growing body of evidence demonstrating that some of these compounds have beneficial properties that may be advantageous in preventing or delaying the onset of disease.

Indeed, several epidemiological studies considering the effect of diet on disease such as, e.g., cancer and cardiovascular disease associated with high cholesterol, have provided leads in the search for naturally-occurring anti-cancer or anti-cholesterol agents. For example, some studies suggest that plant-based diets, rich in whole grains, legumes, fruits and vegetables, may reduce the risk of various types of cancer (Steinmetz et al., Cancer Causes Control. 2:325-357 (1991); World Health Report 2000, World Health Organization, Geneva, Switzerland (2000).

Similarly, other studies report that populations consuming large amounts of fruit and vegetables have a lower incidence of cardiovascular disease and reduced risk of several types of cancer. Such studies have attributed the beneficial properties of diets rich in fruits and vegetables to the presence of naturally occurring compounds, including various vitamins and minerals, and these compounds have been found in a wide variety of plant sources (Rijnkels et al., Cancer Lett., 114:297-298 (1997); Narisawa et al., P.S.E.B.M. 224:116-122 (2000); Miyagi et al., Nutr. Cancer, 36:224-229 (2000); Reddy et al., Carcinogenesis, 2:21-25 (1981); Kawamori et al., Cancer Res. 59:597-601 (1999); Levi et al., Cancer, 36:2115-2119 (2000); Wang et al., Cancer Lett., 98:63-69 (1995); Kim et al., Chemoprevention Rev., 54:259-279 (1996) and; Quereshi et al., Am. J. Clin. Nutr., 53:1021 S-6S (1991)).

Cancer is one of the leading causes of death in adult humans. Cancers of the gastrointestinal tract include some cancers with a particularly high incidence of morbidity. Esophageal cancer is the third most common gastrointestinal malignancy and the sixth most frequent cause of cancer death in the world. Esophageal squamous cell carcinoma (SCC) is one of the most common malignant neoplasms worldwide. The incidence rate of this disease varies dramatically in different geographical areas of the world. The highest incidence areas occur within the “Esophageal Cancer Belt” which includes areas in eastern Turkey, the former Soviet Union, Iraq, Iran, China, Japan, South Africa, and France. More than one-half of all cases of esophageal SCC worldwide occur in China, with approximately 250,000 new cases each year. (Lu, et al., IARC Scientific Publ., 105: 11-17 (1991)). In the United States, approximately 13,300 Americans are expected to die of esophageal cancer in 2004. More than one-half of these deaths will be due to esophageal adenocarcinoma, since this disease now accounts for more esophageal cancer deaths in the United States than SCC.

Esophageal SCC has a complex etiology. In the Western world, tobacco use and alcohol consumption are the major etiological factors for the disease. In the Far East, in addition to the use of tobacco and alcohol, the disease is associated with the intake of salty food and of food contaminated with various mycotoxins, deficiencies in dietary vitamins and minerals, and thermal injuries due to the consumption of hot beverages. Nitrosamine carcinogens in tobacco smoke, in the diet, and those produced in the acidic conditions of the stomach, appear to be important causative agents of esophageal SCC (see for example, Daly, et al., J. Am. Coll. Surg., 190: 562-572 (2000)). Among these is N-Nitrosomethylbenzylamine (NMBA), present in the diet in China, and likely the most potent nitrosamine carcinogen for the rat esophagus. NMBA-induced tumors in the rat esophagus have been used as a model for esophageal squamous cell carcinoma in humans. (Stoner, et al., Toxil. Appl. Pharmacol., 224: 337-349 (2000)).

Esophogeal SCC exhibits a relatively fast growth rate, as compared to other gastrointestinal malignancies. Patients with Esophogeal SCC thus have very poor prognoses. Although surgery, chemotherapy and radiotherapy alone or with combined modality approaches have been utilized for treatment, the overall 5-year survival rate for this disease is still very low, ranging from 5% to 15%, with 75% of patients dying within 1 year of initial diagnosis (Boring, et al., CA Cancer J. Clin., 44:7-26 (1994)). The major ultimate causes of death are typically hematogeneous metastasis to liver and lung and lymph metastasis.

The process of metastasis development is under broad study for seeking effective treatment for malignancies. Angiogenesis is recognized as necessary for metastasis, both for transmission of metastases to other tissues, and for development of tumors in situ. Angiogenesis is the formation of new capillaries from preexisting blood vessels, and angiogenesis along with further vascular development is essential for tumor growth and expansion because it enhances the opportunities for tumor cells to reach the general circulation and metastasize. Those solid tumors of less than 2 mm in diameter can obtain oxygen and nutrient supplies from neighboring blood vessels by simple passive diffusion. Beyond this size, the formation of new vasculature is required for tumor cell growth and survival in order to provide sufficient blood to nourish and oxygenate the tumor cells. Several growth factors have been reported to have angiogenic activity including vascular endothelial growth factor (VEGF), basic fibroblast growth factor (bFGF) and platelet derived growth factors (PDGF). Unlike the other listed angiogenic growth factors, VEGF is a highly specific mitogen (cell division inducing agent) for vascular endothelial cells and induces their proliferation and migration. VEGF is thus regarded as the major angiogenesis factor during carcinogenesis and tumor metastases. There are several isoforms of VEGF, and the VEGF family includes VEGF-A, -B, -C, -D and -E. Differing regulation and tissue distribution suggest that the VEGF isoforms exhibit different roles in angiogenesis. VEGF-A was reported to be overly expressed in human colon cancer. VEGF-C was up-regulated in various human cancers of lung, breast, head and neck, thyroid, stomach, uterus, prostate, colon and ESCC. The expression of VEGF-D was shown to be elevated in human colorectal cancer. For further discussion of the VEGF family and its expression, see: Veikkola, et al., Cancer Res., 60: 203-212 (2000); Easwaran, et al., Cancer Res., 63: 3145-3153 (2003); Kajita, et al., Br. J. Cancer, 85: 255-260 (2001); Ichikura, et al., J. Surg. Oncol., 78, 132-137 (2001); Noguchi, et al., Oncol. Rep., 9(5), 995-9 (2002).

Examination of microvessel density (MVD) in histological sections is a common method by which to estimate the degree of new blood vessel formation, and thus measure the amount of angiogenesis that has occurred. Certain studies have used immunohistochemistry to highlight vascular endothelial cells with antibodies against a number of growth factors and angiogenesis related molecules, for instance, platelet endothelial cell adhesion molecular-1 (PECAM-1, CD31), CD34, CD36, CD105, Ulex europaeus agglutinin 1 (UEA-1) and von Willebrand factor (VWF). In particular, numerous previous studies have demonstrated a positive correlation between VEGF expression and MVD in human esophageal SCC. See Shih, et al., Clinical Cancer Res., 6: 1161-1168 (2000).

Nitric oxide (NO) is a small endogenous biological mediator. NO has many physiological and pathophysiological actions. NO is synthesized in mammalian cells from the conversion of L-arginine to citrulline by a family of three nitric oxide synthase enzymes (NOS; Ref. 13). Historically, NOS enzymes have been classified into two categories: constitutive (nNOS-neuron-produced and eNOS-endothelial cell-produced) and inducible (iNOS). The two constitutive isoforms are calcium-dependent, requiring activation by calmodulin to produce NO and produce only a low level of nitric oxide.

In contrast, the inducible isoform of nitric oxide synthase, (iNOS), is calcium- and calmodulin-independent and, when induced by cytokines or other factors, generates a much higher concentration of NO. Increased NO production is associated with many disorders including cancer. Up-regulation of iNOS has been reported in several types of human cancer including breast, head and neck, lung, colon, melanoma, prostate, skin, and esophageal SCC. Numerous experimental and clinical reports indicate that iNOS mRNA expression is up-regulated in chronic inflammatory diseases as well as in cancer. iNOS protein has been detected in both premalignant and malignant clinical biopsies from the human stomach, colon, lung, esophagus, and prostate; and, increased iNOS activity was observed in human esophagus, colorectal, breast, lung, head and neck, and central nervous system tumors.

Activation of iNOS can lead to the generation of high concentrations of NO. Because NO is a free radical with an unpaired electron, it can donate or accept an electron to become a nitrosonium cation (NO⁺) or a nitroxyl anion (NO⁻), the formation of which lead to nitrosative stress and oxidative stress, respectively. Nitrosative stress can further lead to the formation of nitrosamine carcinogens, deamination of DNA bases and inactivation of DNA repair proteins, all actions contributing to carcinogenesis. For example, iNOS mRNA levels are significantly elevated in NMBA-induced preneoplastic esophageal lesions and in papillomas when compared with normal rat esophagus. Similarly, oxidative stress can lead to the formation of peroxynitrite, which can damage DNA leading to carcinogenesis.

With the correlation of increased iNOS levels with preneoplastic lesions and with advanced cancers, there is a continuing desire to identify methods for eliminating or reducing the activity of such cellular signals that may be involved in the promotion of disease progression from preneoplastic lesions to metastatic cancer. As one example, S,S-1,4-phenylene-bis(1,2-ethanediyl)bis-isothiourea (PBIT) is a selective inhibitor of iNOS; its specificity for iNOS was established in cytokine-induced colorectal adenocarcinoma DLD cells in which it was found to selectively inhibit iNOS but not endothelial cell-produced NOS or neuron-produced NOS. In a study by Rao et al., PBIT suppressed azoxymethane-induced aberrant crypt foci formation, crypt multiplicity, and iNOS activity in the rat colon. Unfortunately the identification of chemopreventive agents that inhibit tumor progression in the esophagus of rats that have been preinitiated with NMBA has proven to be difficult. However, PBIT was shown to inhibit NMBA-induced tumors in the rat esophagus. (Chen, et al., Cancer Res., 65: 3714-3717 (2004).

Prostaglandin endoperoxide synthase (PGHS), also called cyclooxygenase, is an enzyme that catalyzes the formation of prostanoids including prostaglandins A₂, D₂, E₂, F_{2α}, I₂, J₂, and thromboxane A₂.from arachidonic acid. Two isoforms of cyclooxygenase have been cloned: COX-1 and COX-2. COX-1 is constitutively expressed in most mammalian cells and is responsible for a variety of physiological functions. In contrast, COX-2 is preferentially expressed in preneoplastic lesions, in tumors and in response to certain stimuli such as growth factors, tumor promoters, hormones, and cytokines. COX-2 is important for tumorigenesis because prostaglandins, especially prostaglandin E₂ (PGE₂), affect cell proliferation, differentiation, apoptosis, angiogenesis, and metastasis. A correlation has been recognized in the up-regulation of COX-2, VEGF, and iNOS with cancer development in various cancers including colon, stomach, breast, skin, pancreas, lung, head and neck, urinary bladder and in esophageal SCC. See, for example, Eibl, et al., Biochem. Biophysic. Res. Communi., 306: 887-897 (2003); Ichinoe, et al., Histopathology, 45: 612-618. (2004). The expression of VEGF, cyclooxygenase-2 (COX-2) and inducible nitric oxide synthase (iNOS) are thus recognized as critical inducible substances involved in the development and progression of many human cancers including esophageal SCC. Providing a therapeutic substance and or method to reduce the expression of these inducible cancer and carcinogenesis related substances is expected to be effective for slowing or stopping the progression of cellular dysplasia and dysplastic lesions into dangerous metastatic cancers.

One strategy for cancer prevention is chemoprevention, which is defined as the use of either naturally occurring or synthetic dietary constituents to limit cancer initiation and progression. For instance, fruits and vegetable contain many identifiable chemopreventative agents, including carotenoids, isothiocyanaates, monterpenes, flavanoids and catechins. Fruit products are thus widely recognized in the food science art as a source of a number of health promoting phytochemicals. (Johns et al., Recent Advances in Phytochemistry, pp. 31-52, Plenum Press (1997)).

In certain instances, consumption of fresh or preserved fruits and vegetables may be effective for providing a chemopreventative benefit. More commonly, beneficial substances present in fruits and vegetables are present in very small concentrations in the food. Providing for the addition of substances derived from fruits and vegetables in therapeutically effective concentrations would allow for the consumption of beneficial chemopreventative substances without excessively increasing the calorie content or volume of food consumed. Thus, in light of the known correlations between diet and incidence of cancer, there is a need to provide dietary supplements that deliver beneficial phytochemicals at concentrations sufficient to modulate cell dysplasia, reduce cancer incidence and inhibit the progression of precancerous lesions (i.e. dysplastic lesions) to cancer.

For instance, the correlation between diet, smoking and other environmental factors with esophageal cancer suggests that dietary supplements may prove useful for reducing the incidence of cancers of the aerodigestive tract. Given that cancer and cardiovascular disease (e.g., cholesterol-related diseases) are two of the major causes of death in the United States, providing for the identification and concentration of fruit-derived therapeutic compounds which, for example, are useful in treating or preventing such diseases, would be of great benefit to human and animal health.

### BRIEF SUMMARY OF THE INVENTION

Various objects of the invention will, in part, be obvious and will, in part, appear hereinafter. The invention, accordingly, comprises the compositions, methods and system possessing the properties disclosed herein in the summary, the appended claims, and which are exemplified in the following detailed description.

The invention first arose through research to determine whether inclusion of certain commonly consumed fruits in the diet could provide measurable health benefits. The fruits specifically studied included strawberry and caneberries such as raspberry. Using animal models for oral esophageal, colon and skin cancers it was discovered that the inclusion of these fruits and extracts from these fruits in the animal diet could reduce the incidence and/or slow the progression of these cancers. While health benefits have been suggested from the consumption of fruits for decades, embodiments herein disclosed demonstrate specific health benefits for specific families of fruits. The disclosure is further embodied in the recognition that particular fractions of the fruit provide health benefits and by separating the therapeutically beneficial fractions from the rest of the fruit, a composition useful as a practical dietary supplement is produced. Thus, the organic solvent fraction of a fruit extract, as disclosed herein, provides a composition that can be added to the human diet and provide health benefits far exceeding those benefits that could be obtained from eating fruit alone. A preferred embodiment is a product according to process utilizing an ethanol/water fruit extract, thus enriched for particular phytochemical activity, and that phytochemical activity may contribute to the specific health benefits, either alone or in conjunction with other compounds present in the ethanol/water extract. Among the most prevalent compounds in the claimed extract are the anthocyanins, i.e. phenolic compounds, which exhibit among their properties strong antioxidant activity. The beneficial activities of the fruit extracts are correlated with anthocyanin composition.

The disclosure enables the use of a new composition enriched in ability to prevent and treat disease. That composition is derived from the fractionation of fruit pulps with an organic solvent/water mixture, and the beneficial components of the fruit pulp preferentially are accumulated in the organic solvent fraction. The present invention provides novel compounds and therapeutic compositions (e.g., formulations) derived from fruits, in particular berries, and more particularly strawberry and raspberry, as well as novel uses for the compounds and compositions. In particular embodiments, the compounds are formulated as a pharmaceutical, a foodstuff (e.g., added to a foodstuff to enhance its nutritional and/or medical value), or a dietary supplement. In all cases, the compounds and compositions contain, or are enriched for, health promoting components (e.g., antioxidants, carotenoids, phenolic compounds, phytosterols, and associated minerals such as calcium, selenium and potassium) that are useful in treating or preventing a variety of health-related disorders and diseases. In addition, the invention provides methods of efficiently producing berry, e.g., strawberry and raspberry extracts (and fractions thereof) enriched for antioxidant activity (and other desirable components) such that the extracts (or fractions) can be added to foodstuffs or used as a dietary supplement or a pharmaceutical composition.

Accordingly, in another embodiment, the present invention provides a method for treating or preventing a disease in a subject, particularly a malignancy (e.g., a cancer), by administering to the subject (e.g., orally or, when appropriate, by other routes) a therapeutically-effective amount of a compound or composition (e.g., an extract or extract fraction) of the invention. The malignancy can be, for example, metastatic, an aerodigestive tract cancer, or a metastatic aerodigestive tract cancer (e.g., an oral, pharyngeal, laryngeal, esophageal, stomach, or colon cancer). In another embodiment, the present invention provides a method for treating or preventing other diseases or disorders associated with oxidative damage such as skin cancer, cardiovascular disease (e.g., due to high cholesterol, i.e., hypercholesterolemia), neurodegenerative disease (e.g., stroke), immunological diseases or conditions, inflammatory diseases or conditions such as arthritis, dermatological conditions, and opthalmological conditions, in a subject, by administering to the subject a therapeutically-effective amount of a compound or composition of the invention (e.g., an extract or extract fraction of the invention). The compounds or compositions of the invention, when administered to a subject, may also be used to retard aging. The factors contributing to aging being, for example, oxidative mechanisms and compounds, for example, oxidative radicals, which can damage cellular lipids, proteins, and genetic material.

Novel compositions of the invention are derived (e.g., isolated) from, or contain components of strawberry and raspberry fruits, for example, strawberry, blackberry and black raspberry, and combinations thereof. Particular compositions identified by way of the present invention as having significant preventative and therapeutic value include and/or are derived from strawberry and/or raspberry (e.g., black raspberry) fruits which have been, for example, pureed, freeze-dried (referred to as a berry extract), organically extracted (e.g., by solvent extraction of a berry extract, thereby resulting in a berry extract fraction), and combinations thereof. Such berry extracts and fractions thereof, can then be formulated in a variety of manners, such as a dietary supplement, a pharmaceutical, or as an additive to a foodstuff. They may also contain additional desirable compounds such as carbohydrates, some proteins, fiber (e.g., cellulose, lignin), and combinations thereof.

In a related embodiment, the present invention further provides therapeutic compositions containing novel combinations and/or ratios of health-promoting compounds derived (e.g., isolated) from strawberry and raspberry (e.g., black raspberry). Such compounds can be isolated from, for example, strawberry and raspberry (e.g., black raspberry), extracts and/or fractions. By way of non-limiting illustration, such compounds can include antioxidants, vitamins (e.g., vitamin A, vitamin E (tocochromonals), vitamin C (ascorbic acid), folic acid, carotenoids, phenolic compounds, phytosterols, minerals, or combinations thereof.

In another aspect, the invention provides a method for isolating berry extracts, and optionally, fractions thereof, so that the extracts and/or fractions can be administered to a patient or to an animal as a therapeutic agent. In one embodiment, the method involves freeze drying the berries, followed by pulverization into a powder, then exposing the resultant extract to low temperature, and removing an amount of water content, e.g., under a vacuum (e.g., about half an atmosphere, e.g., 380 millitorr, e.g., by sublimation), thereby resulting in a freeze-dried extract enriched for antioxidant activity and other beneficial compounds. In a related embodiment, the berry extract is then exposed to an organic solvent to produce an extract/solvent mixture, and the solvent portion of the extract/solvent mixture is then removed, thereby producing an isolated berry extract fraction substantially free of solvent, e.g., greater than 95% free of solvent, preferably, greater than 99% free of solvent. When the solvent is well tolerated by an animal, e.g., ethanol, the solvent concentration can remain as high as appropriate to deliver the beneficial components to the animal (e.g., fractions in 50% ethanol). Other solvents include dichloromethane, methanol, ethanol, acetone, and combinations thereof, with preferred combinations being about a 1:1 combination of dichloromethane and methanol, about a 1:1 combination of dichloromethane and ethanol, about a 1:1 combination of acetone and methanol, or about a 1:1 combination of acetone and ethanol. Fractions derived from an extract (e.g., a freeze-dried extract) preferably represent at least about 50 to 55% of the starting extract material.

In a related embodiment, the berry extract or extract fraction of the above method is enriched, by about 1-5 fold, preferably 5-10 fold, more preferably by about 10 fold or greater, for antineoplastic activity and the presence of, e.g., one or more of the following: a vitamin (e.g., vitamin A, vitamin E, vitamin C, folic acid), carotenoid (e.g., α-carotene, β-carotene, zeaxanthin, and lutein), a phenolic compound (e.g., ellagic acid, ferulic acid, coumaric acid, anthocyanidins such as cyaniding and quercetin, pelargonidin, and analogs thereof), a phytosterol (e.g., β-sitosterol, campesterol, kaempferol, stigmasterol, and analogs thereof), and a mineral (e.g., calcium, magnesium, potassium, zinc, and selenium).

A preferred embodiment is an isolated organic solvent fruit extract fraction having a therapeutically effective amount of activity in modulating undesired signal transduction activity of one or more of the molecule NF-Kβ, the molecule AP-1, the molecule Akt, the molecule iNOS, and the molecule VEGF, and therefore useful for reducing the frequency, duration or severity of a neoplastic disease or condition in a subject, said fruit extract being derived from a plant of one or more of the genera Fragaria or Rubus. Preferably the fruit extract fraction is from one or more of strawberry, raspberry, red raspberry, black raspberry, ligonberry, cloudberry, blackberry and blackberry. A further embodiment is wherein said amount of activity useful for modulating undesired signal transduction activity is present in an amount at least about 100% greater than present in a native fruit, or the undisrupted fruit.

Another preferred embodiment is a dietary supplement comprising a consumable supplement fortified with a fruit extract fraction capable of delivering a chemopreventative agent to the gastrointestinal tract, and where the free sugars such as sucrose and or fructose have been diminished, with less than one half the free sugar of the native fruit.

A further embodiment is a method for treating or preventing a disease or condition in a subject comprising the step of administering to said subject a therapeutically-effective amount of a foodstuff, dietary supplement or pharmaceutical composition fortified with an organic solvent fruit extract fraction having a therapeutically effective amount of activity in modulating undesired signal transduction activity.

Yet another embodiment is a fruit extract fraction product prepared according to process comprising,

a) harvesting fruit from a plant of one or more of the genera Fragaria or Rubus and chilling said fruit to about 4° C. within four hours;

b) physically disrupting an amount of chilled fruit;

b) maintaining the disrupted fruit at a low temperature of less than about 4° C. until fractionated;

c) removing an amount of water content from the disrupted fruit by sublimation under a vacuum of less than about 400 millitorr;

d) adding to the fruit extract an organic solvent to produce an extract/solvent mixture; and

e) removing the solvent portion of the extract/solvent mixture thereby producing isolated fruit extract fraction substantially free of solvent,

wherein the activity of the fruit extract fraction is has at least about a three fold increase in anti-dysplastic activity compared to the undisrupted fruit as measured by the activity of the fruit extract fraction in inhibiting one or more of AP-1, NFKB, Akt, COX-2, and VEGF.

Furthermore, the said vacuum of the above process may be increased to at least about 200 millitorr, and the low temperature is preferably less than about −20° C.

In another embodiment, the extracts of the methods (or fractions thereof) enriched for, e.g., antioxidant activity, are suitable for use in a foodstuff, a dietary supplement, or a pharmaceutical composition. Accordingly, the extracts of the invention (or fractions thereof) can be used in the treatment of a subject in need of an antioxidant therapy or having an antioxidant responsive disease or condition, such that treatment is achieved. The invention provides a practical dietary supplement, by providing for an enriched fraction of the original fruit, enabling subjects to consume larger quantities of beneficial composition without consuming a large mass of fruit. Furthermore, the extract fraction can be added to pharmaceutical preparations in a manner that would be entirely impractical with whole fruit or fruit juice.

Other features and advantages of the invention will be apparent from the following detailed description and claims.

### BRIEF DESCRIPTION OF THE DRAWINGS

FIG. 1 shows interactions of the fruit extract composition with cell signaling pathways;

FIG. 2A shows a flow chart of a method for extracting desirable fractions from freeze-dried fruits. Fractions containing desirable compounds that can be further partitioned are also shown. Abbreviations: RU=black raspberry (Rubus occidentalis); FA=strawberry;

FIG. 2B shows additional chromatographic steps and alternative fractionation approaches for extracting desirable fractions;

FIG. 3 shows the inhibition of oral cancer in the hamster cheek pouch (HCP) in hamsters fed diets containing 5% and 10% black raspberry extracts as compared to controls;

FIG. 4 shows the inhibition of BPDE-induced activator protein-1 (AP-1) activity in mouse epidermal cells (i.e., JB-6 clone 41) transfected with AP-1 (P+1-1 cells) treated with black raspberry extract fractions as compared to controls;

FIG. 5 shows the dose-dependent inhibition of BPDE induced AP-1 activity in cells (P+1-1 cells) treated with the methanol extract fraction of black raspberries;

FIG. 6 shows the inhibition of BPDE-induced NFκB (b) activity in cells (mass1 cells) treated with black raspberry extract fraction (RU-ME);

FIG. 7 shows the dose-dependent inhibition of BPDE induced NFκB activity in cells (mass1 cells) treated with the methanol extract fraction of black raspberries;

FIG. 8 shows the inhibition of BPDE induced activation of MAPKs and IκBα phosphorylation and degradation in mouse epidermal cells (JB-6 clone 41) treated with black raspberry extract fraction (RU-ME) as compared to controls as determined by immunoblot;

FIG. 9 shows that the inhibitory activity of the black raspberry extract fractions is independent of BPDE induced p53-dependent transcription activity in mouse epidermal cells (JB-6 clone 41) transfected with p53 (mass1 cells);

FIG. 10 shows a chromatogram obtained upon HPLC analysis of the methanol fraction of lyophilized black raspberry extracts;

FIG. 11 shows the chemical structure of several active compounds identified in the lyophilized black raspberry extracts of the invention, i.e., cyanidin, quercetin, pelargonidin, and kaempferol;

FIG. 12 shows the generalized chemical structure of anthocyanins, including the substituents present on the anthocyanins cyanidin, and pelargonidin, among others;

FIGS. 13-15 show LC-ESI-MS chromatograms obtained upon analysis of the methanol fraction of lyophilized black raspberries for the presence of cyanidin, quercetin, pelargonidin, and kaempferol, and sugar conjugates thereof;

FIG. 16. shows changes in VEGF-C mRNA expression in rat esophagus resulting from treatment with fruit extract fractions;

FIG. 17 shows microvessel density staining pattern in subepithelial plexus of normal epithelium (A), NMBA-treated epithelium (B) and NMBA+BRB-treated epithelium (C);

FIG. 18 shows an immunoblot demonstrating that Rubus fractions inhibit B[a]PDE-induced AP-1 activation through inhibiting activation of the PI-3K/Akt pathway;

FIG. 19 shows the identified cell metabolic targets of the isolated composition;

FIG. 20 shows the correlation of VEGF, COX-2 and iNOS in NMBA-treated rat esophagus (A) and NMBA+BRB-treated rat esophagus (B);

FIG. 21 shows the suppression of iNOS mRNA in preneoplastic lesions (A) and shows suppression of the expression of COX-2 mRNA expression following fruit extract treatment (B and C).

### DETAILED DESCRIPTION OF THE INVENTION

The present invention is based on the identification of therapeutic berry extracts, for example, strawberry and black raspberry extracts, and fractions or compounds isolated from the extracts (e.g., a berry extract fraction), having novel therapeutic and/or health promoting value. In particular, therapeutic berry extracts of the invention (and fractions thereof) are shown herein to exhibit significant anti-cancer and anti-hypercholesterolemia activity when administered to a subject in vivo and when tested in vitro.

In a particular embodiment, therapeutic methods of the invention employ physically disrupted berry fruit, preferably a puree free of cap stems, which is freeze-dried to produce a berry extract substantially free of water content and is enriched for a number of health promoting compounds and exhibits e.g., significant anti-cancer properties when administered to a subject. Moreover, a variety of particular health promoting compounds derived from prepared berry have been identified and are discussed below.

In another embodiment, therapeutic methods of the invention employ berry products (e.g., extracts, or fractions thereof) which are novel sources of compounds having significant therapeutic value, in, for example, the prevention or treatment of cancer, particularly, aerodigestive cancers. In addition, as described herein, a subset of these berry derivatives are also enriched with compounds suitable for treating cardiovascular disease related to, for example, high cholesterol (hypercholesterolemia).

In another embodiment, therapeutic methods of the invention employ compounds derived from berry extracts which, as shown herein, have anticancer activity, e.g., reduced conversion of preneoplastic lesions (dysplasia) to cancer, e.g., when prepared in concentrated form and administered to a mammal in vivo. For neoplasia, malignancies and cancers, particularly cancers of the aerodigestive tract, and more particularly esophageal cancer and colon cancer, chemoprevention could be an important strategy, because high-risk populations for this disease can be identified. Moreover, the chemopreventative agent can be delivered in close proximity to those neoplastic and preneoplastic cells which are the target for treatment. Absorption into the bloodstream of the subject patient may be desirable, but certain chemopreventative agents that are not readily absorbed may still provide therapeutic benefit to the aerodigestive tract.

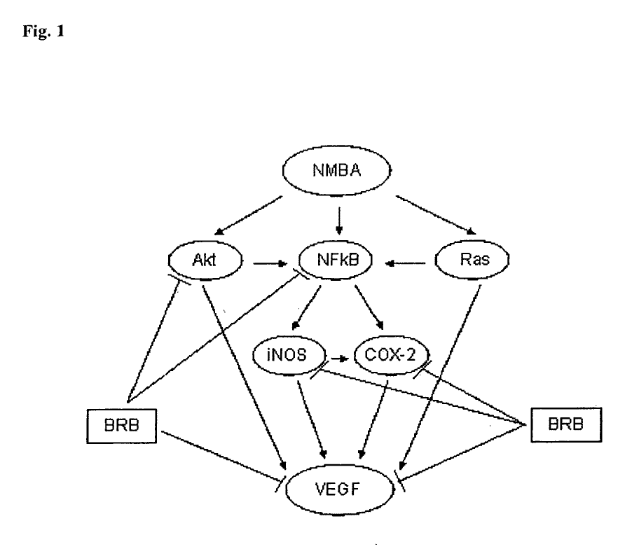

Specific prophylactic and therapeutic health benefits are provided by an organic solvent fraction/water extract of fruits. As shown in FIG. 1, the fruit extract from black raspberry, for instance, interacts with a number of components of the cell signaling mechanisms of the animal body. As described, activation of angiogenesis by induction of VEGF (vascular epidermal growth factor) is an important step in the transition from cell dysplasia to neoplasia and potentially cancer. The fruit extract from black raspberry (BRB) may act directly upon the activation of VEGF. Moreover intermediate components of a cell signaling cascade such as iNOS and COX-2 are also affected by BRB. Two components of the cell signaling systems, that may act further upstream Akt and NFKB are shown herein to be down modulated by the fruit extract from black raspberry, BRB. The activity of carcinogenic agents, such as NMBA, on Akt and NFKB are demonstrated herein to be blocked by BRB. In a demonstration that the activity of

BRB fractions is specific, it is also shown that the RAS signaling pathway is not inactivated by BRB and or BRB fractions. Accordingly, the identification of particular beneficial compounds in berry extracts and derivatives thereof has allowed for the development of convenient methods and compositions (e.g., formulations) for administering therapeutic compounds to treat or prevent particular diseases. Moreover, the therapeutic compounds and compositions described herein have the additional advantage of being readily manufactured into palatable forms (e.g., as foodstuffs such as juices and food bars or as dietary supplements) for convenient oral administration.

Methods for obtaining and preparing the berry extracts of the invention, identifying (e.g., characterizing) and obtaining therapeutic components of the products, evaluating biological activity in vitro and in vivo of the products and components, and methods of using the products and novel compositions containing the products or combinations of components isolated from the products, are discussed in the following subsections.

Methods for Preparing Berry Extracts and Fractions Thereof

Berry extracts of the invention (and fractions thereof) may be isolated from whole berry, preferably freshly harvested berries, using any suitable art recognized method. It is recognized that certain of the important chemoprotective compounds or complexes have reduced stability at ambient temperatures in the whole fruit or fruit pulp. Thus, chilling the fresh fruit as soon as practicable is preferred. Preferably, the fruit is cooled with an ice bath or cold water wash or other method to about 4° C. within four hours of harvest, even more preferably within about 2 hours of harvest. Preferred derivatives include berry extract, or fractions thereof, that optionally, have been freeze-dried. The berries may be freeze-dried using any art recognized method. In a particular method, the berries are freeze-dried by first physically disrupting the berries resulting in a puree which is then further processed to be substantially free of impurities or undesired solids, e.g., stems. The puree is then poured into a shallow vessel and quickly exposed to low temperature, i.e., flash frozen, for example at −20° C. or lower, preferably under a vacuum for removal of water content (lyophilization). The resultant berry extract, as compared to the native fruit by weight, is typically enriched for, e.g., antioxidant activity, antioxidant compounds, and other compounds described herein, by a factor of at least about 1-5 (i.e., ˜100-500%), preferably, by a factor of at least about 5-10 (i.e., ˜500-1000%), more preferably by a factor of at least about 10 or more (i.e., ˜1000% or more).

The resultant extract (i.e., lyophilized) may be, optionally, fractionated by adding an organic solvent to produce an extract/solvent mixture, and removing the solvent portion of the extract/solvent mixture such that an isolated berry extract substantially free of solvent results. By selection of particular solvents, as described below, fractions enriched for particular compounds with health promoting activities, can be obtained. In one embodiment of the extraction method, an isolated berry extract results that is suitable for use in a foodstuff, dietary supplement, or pharmaceutical composition.

In all cases, the berry extracts or fractions are preferably obtained in a form suitable for use in a foodstuff, dietary supplement, or pharmaceutical composition. Further, it is understood that with regard to any of the techniques for preparing a berry extract or derivative described herein, it may also be desirable to avoid exposing the derivative, or component thereof, to oxygen by, e.g., protective blanketing of the derivative or component with an inert gas (e.g., carbon dioxide or nitrogen gas), or by, e.g., exposing the derivative or component, where appropriate, to low temperature, a ‘stabilizer, or a combination of these conditions.

Berry Extracts and Fractions Thereof

It has been recognized by layman and medical professionals for quite some time that the consumption of fruits as part of the diet may provide a medical benefit. An old adage recommends the daily consumption of a fleshy fruit from a member of the Roseaceae family as a means of avoiding the need for medical attention. Nonetheless, the present disclosure provides specific teachings as to how consumption of particular components of fleshy fruits may be used in the prophylaxis against and therapeutic treatment of particular medical conditions. Artisans have recognized the presence of a variety of compounds with potential to provide a benefit, and these so-called “healthy” compounds include such previously identified compounds such as vitamins and anti-oxidants present in fruit, and in particular fruits of the Roseaceae family, such as strawberry (Fragaria sp.), ligonberry (Eriobotrya japonica), blackberry (e.g., Rubus. fructicosus) and raspberry (e.g., Rubus occidentalis). The Rubus genus contains a number of wild and cultivated species of similar botanical and biochemical characteristics. Other important Rubus species include cloud berry (Rubus chamaemorus), and salmonberry (Rubus spectabilis). Disclosed herein is a method to extract from fruits certain compositions useful for treating particular diseases or conditions, such as cell dysplasia and oral cancer. As part of the disclosure, several berry extracts, including strawberry and black raspberry (including fractions thereof) are shown to possess health promoting antioxidant activity and other various beneficial compounds. These berry extracts were analyzed using both chemical analysis and bioactivity assays as described herein. In addition, a number of berry extract fractions were also studied for their in vitro and in vivo therapeutic activity and analyzed for health promoting compounds (see Tables 1-3).

Thus, while it was known, for instance, that pureed fruit from several species of the genus Rubus possess antioxidant activity and contain vitamins such as Vitamin C, along with anthocyanins and other phenolics, disclosed herein are particular fractions of the processed fruit that are preferred for use in the diet of subjects. Prior to the present disclosure, there was a dearth of specific knowledge regarding the in vivo activity of compounds derived from the fruits of Rubus or other Roseacean plants in the animal body. The disclosure provides specifically identified activity useful for modulating a number of components of cell signaling pathways, including Akt, NFKB, iNOS, COX-2 and VEGF. With the present disclosure, it is now clear that certain compositions derived from these fruits are useful for treating or preventing particular diseases or conditions, such as cardiovascular disorders, cell dysplasia, skin cancer, oral cancer, esophageal cancer, and colon cancer.

Accordingly, by way of the studies described herein, it was shown that particular berry extracts are novel sources of therapeutically beneficial phytochemical activity (e.g., as measured by the oxygen radical absorbance capacity (ORAC) of the extracts) as well as for compounds such as a vitamin A, vitamin E (tocochromanols), vitamin C (ascorbic acid), folic acid, carotenoids, anthocyananins and other phenolic compounds, phytosterols, minerals, and combinations thereof. The berry extracts prepared as described herein provide several advantages over currently known sources of such therapeutically beneficial compounds including, for example, remarkably high levels of antioxidant activity as well as the presence of many desirable components. A number of means may be used to standardize and or quantify the predicted chemotherapeutic activity of the fruit extract fractions disclosed herein. One such means is through use of ORAC values as a marker for concentration of beneficial components. While antioxidant activity alone may not be the only, or even primary beneficial activity of the fruit extract fractions, ORAC values may be used to gauge the relative concentrations of co-eluted constituents. Thus and ORAC value of 5 or greater is desired, as is a value of 10, and preferably 15 or even 20. It is recognized by artisans that increasing concentration of active ingredient may be valued for certain applications, such as for pharmaceuticals, such as an ORAC of 20, while a lower ORAC of, for instance 5, may be valued for use in foodstuffs. Accordingly, the berry extracts of the invention, or components thereof, can be used in foodstuffs, dietary supplements, and pharmaceutical compositions.

Accordingly, in one embodiment provided, a fruit extract, for example, a strawberry or black raspberry extract, or a composition comprising one or more components of such an extract, as listed, respectively, in Tables 2 and 3, which promotes health in a human or other animal. The berry extracts or composition derived therefrom are also preferably substantially enriched for phytochemical activity, including for antioxidant activity, for example, possessing a high value for oxygen radical absorbance capacity (ORAC) as shown in Table 1. The berry extracts or composition derived therefrom also can contain one or more exogenous (i.e., externally added) compounds to further enhance the therapeutic value of the berry extracts or composition derived therefrom, for example, by acting in synergism with one or more native components of the berry extract.

The strawberry and black raspberry extracts of the invention can contain one or more of the following compounds: vitamins (e.g., vitamin A, vitamin E, vitamin C, and folic acid); carotenoids (e.g., α-carotene, β-carotene, zeaxanthin, lutein); phenolic compounds (e.g., ellagic acid, ferulic acid, anthocyanins, such as cyanidin and pelargonidin, quercetin, kaempferol, and analogs thereof); phytosterols (e.g., β-sitosterol, campesterol, and stigmasterol, and analogs thereof); and minerals (e.g., calcium, magnesium, potassium, zinc, and selenium). Certain of the beneficial compounds may be present in small quantities relative to the entire composition, yet exert a substantial beneficial effect. For instance the presence of antioxidant vitamins may confer a therapeutic benefit that enhances the therapeutic benefit conferred by a substance with other mode of action, for example an anthocyanin capable of modulating gene expression in a beneficial manner. In addition, exogenous compounds, such as other vitamins (e.g., vitamins underrepresented) and/or chemotherapeutic agents, can be added to the berry extracts of the invention and compositions derived therefrom, to achieve a synergistic effect.

In addition, the fruit extracts of the invention contain high levels of antioxidant activity as measured by the oxygen radical absorbance capacity (ORAC) of the extracts, and are enriched for certain classes of anthocyanins. In particular, black raspberry extracts are especially enriched for such antioxidants, particularly the anthocyanins. Accordingly, the berry extracts have a high antioxidant activity (in addition to other properties discussed herein).

The therapeutic benefit of the antioxidant activity and other compounds of the extracts is further disclosed and summarized under the following subsections.

Antioxidant Activity

An important activity found in the berry extracts of the invention is antioxidant activity, e.g., as determined by the oxygen radical absorbance capacity (ORAC) value found for each extract. Thus, the berry extracts of the invention (and fractions thereof) have the advantage of being potent delivery systems for antioxidants. Antioxidants, as discussed below, include, e.g., vitamin E, vitamin C, and phenolic compounds. While the beneficial activities of the disclosed fruit extracts are not believed to be due solely to the enrichment for antioxidants such as vitamins, the inclusion of antioxidants such as vitamins may contribute to the activity and or bioavailability of the compounds providing beneficial activity.

Vitamins

The berry extracts of the invention also contain vitamin A which generally includes any member (or combination thereof) of a family of fat-soluble vitamins such as retinol, retinal, and retinoic acid. These compounds play an important role in vision, bone growth, reproduction, cell division and differentiation, immunoregulation, and lowering cancer risk.

The berry extracts of the invention also contain vitamin E which generally comprises tocochromanols (a class of compounds that includes tocopherols and tocotrienols). A large body of research has shown the importance of tocopherols and tocotrienols in the defense against numerous biological disorders.

Accordingly, the berry extracts of the invention and compositions derived therefrom (e.g., fractions rich in vitamin E) can be used to treat respiratory, inflammatory, neurological, dermatological, opthalmological, and gastroenterological diseases. Surprisingly, the amount of vitamin E (tocochromanols) determined to be in the berry extracts of the invention is present at high levels in both strawberry and black raspberry extracts (respectively, 5-6 mg/100 gm; ˜11 mg/100 gm).

The use of vitamin E as an anticarcinogenic agent has been recognized for a number of years (Haenszel et al., Int. J. Cancer, 36:43-48 (1985); Menkes et al., N. Engl. J. Med., 315:1250-1204 (1986); Stahelin et al., Ann. NY Acad. Sci., 570:391-399 (1989)). In addition, in vitro and in vivo studies, including human studies, have demonstrated that vitamin E interferes with the development of carcinogenesis that results from exposure to various environmental factors known to enhance oxidant stress (Borek et al., In, Mechanisms of cellular transformation by carcinogenic agents, New York, Pergamon (1987), Borek et al., In, Medical, biochemical and chemical aspects of free radicals, Amsterdam, Elsevier, (1989); Borek et al., Proc. Natl. Acad. Sci. USA, 83:1490-1494 (1986); Proc. Natl. Acad. Sci. USA, 88:1953-1957 (1991)). (Ames et al., Science 230:271-279 (1987); Doll et al., J. Natl. Cancer Inst. 66:1193-1194 (1981): Greenwald et al., Cancer 65:1483-1490 (1990); Menzel et al., J. Agr. Food Chem., 20:481-486 (1972)).

The berry extracts of the invention also contain vitamin C (ascorbic acid) which can function as an antioxidant. Vitamin C is also useful for promoting healthy teeth and gums, absorption of iron, maintenance of connective tissue and the immune system.

Folic Acid

Berry extracts of the invention also contain measurable levels of folic acid which acts a coenzyme (with other vitamins (vitamins B-12 and vitamin C) in the metabolism of proteins and in the synthesis of new proteins) and is necessary for the production of red blood cells and the synthesis of DNA, tissue growth and cell function. Adequate levels of folic acid are required to prevent neural tube defects during human embryogenesis.

Carotenoids

Berry extracts of the invention also contain measurable levels of carotenoids. Typical carotenoids found within the berry extracts of the invention include α-carotene, β-carotene, zeaxanthin, and lutein. The health promoting effects of the carotenoids of the invention include reducing the risk of developing several kinds of cancer, including skin, oral, stomach, colorectal, esophagus, larynx, and lung cancer.

Phenolic Compounds

Berry extracts of the invention also can contain one or more simple phenolic compounds, such as ellagic acid, ferulic acid, and coumaric acid (but also, e.g., hydrobenzoic acid, hydroxycinnamic acid), complex phenols such as flavonoids (e.g., anthocyanidins), anthocyanins (e.g., cyanidin, pelargonidin), quercetin, kaempferol, and analogs thereof), flavanols, flavan-3-ols, and/or tannins). Such phenolic compounds can act as potent antioxidants and, therefore, can prevent or delay oxidation reactions which cause various diseases.

Accordingly, the berry extracts of the invention and compositions derived therefrom (e.g., certain extract fractions) can be used as antioxidants. For example, they can inhibit lipid peroxidation, scavenge free radicals and active oxygen, inactivate lipoxygenase, and chelate iron ions. Moreover, epidemiological studies have demonstrated that the consumption of phenolic compounds is associated with a reduced risk of cancer. Accordingly, the berry extracts of the invention and compositions derived therefrom (e.g., fractions rich in phenolic compounds) can be used to prevent cancer with few side effects.

In particular, the black raspberry and strawberry extracts of the invention contain significant quantities of various polyphenols including ellagic acid, ferulic acid, and multiple anthocyanins. Ellagic acid alone has demonstrated inhibitory effects against skin, lung, liver, esophagus and colon cancer in animals. In addition, ellagic acid activates Hageman factor (involved in blood clotting); inhibits replication of certain DNA viruses such as adenovirus and herpesvirus; inhibits enzymes involved in the synthesis of retroviruses such as HIV (AIDs) virus; inhibits the bioactivation and stimulates the detoxification of certain chemical carcinogens; scavenges the ultimate carcinogenic metabolite of benzo(a)pyrene, a ubiquitous environmental carcinogen; exhibits antimutagenic activity in the AMES mutagenesis assay; and, has therapeutic effects against tumors in animals. In addition, ellagic acid is a strong antioxidant. Ferulic acid also exhibits antimutagenic and antioxidant activity. The anthocyanins impart color to berries and are polyphenols that exhibit measurable antioxidant activity, along with additional cell signaling activities. In general, the darker the fruit, the higher the levels of anthocyanins present and the higher the associated antioxidant activity.

Phytosterols

In particular, the berry extracts of the invention can contain one or more phytosterols (plant sterols), including, but not limited to, β-sitosterol, campesterol, and stigmasterol, and analogs thereof.

Phytosterols have been shown to inhibit the absorption of cholesterol from the intestine, and decrease blood serum cholesterol. It has been proposed that, in the intestine, phytosterols act by reducing the solubility of cholesterol in the lipid and micellar phases with a consequential decrease in cholesterol absorption. Plant sterols are also reported to inhibit colon cancer and breast cancer development.

Accordingly, the berry extracts of the invention and compositions derived therefrom (e.g., fractions rich in phytosterols) can be used, for example, in the treatment of patients with cardiovascular disease or as chemopreventative agents against colon cancer and breast cancer.

Minerals

Berry extracts of the invention also contain high levels of minerals. Typical minerals found within the berry extracts of the invention include calcium, magnesium, potassium, zinc, and selenium. The health promoting effects of minerals found within the extracts of the invention include, for example, reducing osteoporosis and cancer risk (calcium), maintaining electrolyte balance (magnesium and potassium), maintaining immune system function (zinc), and reducing cancer risk (selenium).

Methods for Isolating, Identifying, and Analyzing Specific Components from Berry Extracts

To isolate and analyze constituent therapeutic components (compounds) from the berry extracts of the invention, a variety of art-recognized techniques and assays can be employed. For example, phenolic compounds of the strawberry and raspberry extracts and derivatives of the invention can be analyzed and extracted using HPLC analysis and solvent extraction, respectively. The isolated extracts can be dissolved in an organic solvent, for example, methanol (or ethanol, which can be administered to animals, e.g., humans) and then extracted with a methanol/water solution (or ethanol/water) followed by centrifugation. The extract can then be dried, and the residue can be resuspended in methanol/water for HPLC analysis.

Other components of the extracts, for example, carotenoids, phenolic compounds, phytosterols can be extracted and analyzed using, for example, thin layer chromatography and high-performance liquid chromatography. For example, the material can be fractionated on thin-layer chromatography (TLC) plates where the individual bands that are subsequently resolved can be scraped and extracted with a chloroform/methanol solvent. These resultant samples can then be analyzed using, e.g., gas and high-performance liquid chromatography (HPLC).

Such isolated components, which can be separated as “value added” fractions (e.g., fractions having therapeutic value), are typically rich in at least one beneficial component identified from the berry extracts or factions thereof described herein. These isolated components or fractions may be further combined to provide a composition rich in more than one component or, e.g., a desired combinations thereof.

In addition, a particular formulation intended for the treatment or prevention of a particular disease or condition may be formulated to be rich in those components having a therapeutic effect on the disease or condition (e.g., associated with affecting a change in any of the mechanisms associated with that particular disease or condition). For example, a formulation suitable for administering to a subject with cancer is preferably rich in berry extract-derived components having antioxidant activity and other anti-cancer properties, whereas a formulation for administering to a subject with cardiovascular disease (e.g., hypercholesterolemia) is preferably rich in phytosterols. A subject with a dietary need, may be administered a formulation rich in, for example, beneficial vitamins or minerals.

Methods for Evaluating Therapeutic Properties of Berry Extracts And Components Derived Therefrom

In another embodiment, the strawberry or raspberry extracts of the invention, and compositions derived therefrom, can be tested for their in vivo therapeutic effect by administering (e.g., orally) the extracts or compositions in a suitable form (e.g., as a food stuff, dietary supplement, or pharmaceutical composition) to a human or other animal, and then observing the physiological effect (e.g., compared to a control). The human or animal can be, for example, suffering from a disease or condition, such as those described herein (e.g., cancer or hypercholesterolemia). Thus, a reduction in the physical symptoms of the disease can be measured as an indication of the therapeutic efficacy of the strawberry or raspberry extracts or compositions derived therefrom.

In another approach for evaluating anti-tumor activity, strawberry or raspberry extracts of the invention or compositions derived therefrom (e.g., a fraction thereof) can be used in a controlled animal study where tumors are induced in the animal via diet (or by other appropriate routes such as injection, e.g., by intraperitoneal, subcutaneous, or intravenous injection), by applying a chemical tumor promoter to the skin, or by the implantation of tumor cells in the presence or absence of the test agent. Various assays, such as those described below, can then be used to examine the progression of carcinogenesis in the presence or absence of the administration of the extracts or compositions of the invention.

The health promoting properties of berry extracts of the invention and compositions derived therefrom also can be evaluated using a variety of art-recognized cell-based assays. For example, the antioxidant effects on cells caused by exposure to a berry extract of the invention or a composition derived therefrom can be determined by an oxygen radical absorbance capacity (ORAC) assay or electron spin resonance technology as described herein. Typically, the extracts of the invention have enriched antioxidant activity as measured by either of these technologies.

Methods of Use

Treatment of Cancer

In one embodiment, a berry extract of the invention and compositions derived therefrom (particularly those having antioxidant activity) can be administered to a human or other animal to treat or prevent a variety of cancers. In particular, the extracts of the invention are especially well-suited for inhibiting the development of cancers of the aerodigestive tract in animals and humans such as oral, laryngeal, pharyngeal, esophageal (squamous cell carcinoma and adenocarcinoma), stomach, and colon cancer. Other disease indications include preneoplastic lesions in humans such as epithelial dysplasia of the esophagus, development of Barrett's esophagus, oral leukoplakia and erythroplakia, and colonic polyps. The extract and compositions derived therefrom also can be administered in combination with other anti-cancer agents. In particular, the berry extracts of the invention and compositions derived therefrom can be administered with other nutrients, chemotherapy, and/or radiotherapy for the treatment of, for example, an aerodigestive cancer.

Other chemopreventive agents suitable for coadministration for inhibiting development of tumors, e.g., tumors of the oral cavity, when administered before, during, or after initiation by chemical carcinogens include glutathione, beta-carotene, limonin, retinyl acetate, Ocimum sanctum, diallyl sulfide, vitamin E, protease inhibitors from soybeans, ibuprofen, green coffee beans, green tea polyphenols, curcumin, quercetin, and mint. Of these, beta-carotene, retinyl acetate, Ocimum sanctum, diallyl sulfide, retinoids, protease inhibitors, green tea, curcumin, and similar synthetic compounds are suitable for preventing tumor formation when given post-initiation, i.e., after exposure to a chemical carcinogen.

The inhibition of tumor development by fruit or berry extract including the extracts from raspberry, black raspberry, red raspberry, and strawberry fruits derived from plants of the genera Rubus and Fragaria, fruits derived from plants of the Roseaceae family, and the fractionated constituents of these extracts, including ethanol/water or alcohol/water extract fractions are believed to function through a variety of mechanisms, including for instance, through the suppression of DNA adduct formation; inhibition of cell proliferation; down-regulation of COX-2; down-regulation of iNOS; and down-regulation of transcription factor c-Jun (a component of the AP-1 complex), down regulation of VEGF, and a variety of other regulatory genes. In fact, the fruit extract as disclosed exhibit genome-wide effects on the modulation (e.g., down- or up-regulation) of genes associated with cancer development.

An important embodiment of the invention is an isolated organic solvent fruit extract fraction having a therapeutically effective amount of activity in modulating undesired signal transduction activity. Undesired signal transduction activity may be considered as shown in FIG. 1, wherein chemopreventative benefit may be derived by inhibiting (down-regulating) signal transduction components such as that arising from the molecule NF-Kβ, the molecule AP-1, or its subcomponent the molecule c-Jun, the molecule Akt, the molecule iNOS, and the molecule VEGF. Such modulation of undesired signal transduction activity is believed to be useful for reducing the frequency, duration and or severity of a neoplastic disease or condition in a subject patient. With evidence herein disclosing such as benefit when the said fruit extract is derived from a plant of one or more of the genera Fragaria or Rubus.

The biodirected fractionation studies disclosed herein identify several of the most active inhibitory components. Several BRB extracts and anthocyanins have been administered in JB6 CI 41 mouse epidermal cell lines and RE-149 DHD rat epithelium cancer cell lines, and such model systems are predictive of in vivo biological activity. As further discussed below, the BRB methanol fraction inhibits benzo(a)pyrene-7,8-diol-9,10-epoxide (B(a)PDE)-induced transactivation of transcription factor AP-1 and nuclear factor κB (NFκB, NFKB) activity in JB6 CI 41 cell lines; BRB ethanol extracts inhibited cell proliferation in RE-149 DHD cell lines; and the anthocyanins, cyanidin-3-O-glucoside and cyanidin-3-O-rutinoside derived from BRB ethanol extracts, suppressed the expression of COX-2 and iNOS in RE-149 DHD cell lines. A further embodiment is that even the unpurified components of the BRB ethanol extracts are believed to be safe for human consumption, being derived from a consumable foodstuff using consumable extraction solvents.

Demonstration of Anti-Cancer Properties of an extract from Rubus occidentalis.

The anti-cancer properties of the fruit extracts, and particularly the black raspberry extract according to the present disclosure is demonstrated both by in vivo animal studies in vitro cell based studies and studies involving the modulation of gene expression in response to delivery of a composition comprising the fruit extract fraction.

Briefly, freeze-dried black raspberries prepared as described above were evaluated for their ability to inhibit chemically-induced tumors in rodents. Using the rat model of squamous cell carcinoma of the esophagus, freeze-dried black raspberries added at 5% and 10% of the diet 2 weeks before, during, and after subcutaneous administration of the carcinogen, N-nitrosomethylbenzylamine (NMBA) caused significant reductions in esophageal tumor multiplicity of 39 and 49%, respectively. These reductions in tumor multiplicity are very similar to what was obtained with freeze-dried strawberry extracts. In addition, black raspberry extracts added at 5% and 10% of the diet were shown to influence the metabolism of NMBA to DNA damaging species as indicated by the observation that they reduced the formation of O⁶-methylguanine adducts in esophageal DNA by 73 and 80%, respectively. When added at 5% and 10% of the diet following subcutaneous treatment of rats with NMBA, the black raspberries significantly reduced the formation of both premalignant lesions (i.e., low- and high-grade dysplasia) and the number of esophageal tumors per rat by 62 and 43%, respectively. In addition, the berries were shown to reduce the rate of proliferation of premalignant cells as evidenced by significant reductions in the percentage of cells stained positively for proliferating cell nuclear antigen (PCNA). Thus, one mechanism by which black raspberries inhibit tumor progression in the rat esophagus is by reducing the rate of growth of epithelial cells in the esophagus of NMBA-treated animals.

In another study, freeze-dried black raspberries were evaluated for their ability to inhibit the progression of chemically-induced cancer in the rat colon. Rats were given intraperitoneal injections of the colon carcinogen, azoxymethane, once per week for two weeks. One day after the final injection, rats were administered 2.5%, 5%, and 10% freeze-dried black raspberry extracts in the diet. After 33 weeks of dietary administration, 2.5, 5 and 10% black raspberry extracts, reduced total colon tumor numbers (adenoma and adenocarcinoma) by 42, 45, and 71%, respectively. In addition, in the same treatment groups, the number of adenocarcinomas decreased by 28, 35, and 80%, respectively. This is very significant because adenocarcinomas represent malignant tumors in the colon of rats and, also, of humans. All reductions in tumor number were statistically significant. This study also revealed that urinary 8-hydroxydeoxyguanosine (8-OHdG) levels were reduced by 73, 81 and 83%, respectively, in rats administered 2.5, 5 and 10% berry extracts in the diet. Therefore, black raspberry extracts also modulated an important marker of oxidative stress in azoxymethane-treated rats.

In another study, the anti-oral cancer properties of the black raspberry extracts of the invention, and fractions derived therefrom, were examined. In particular, the hamster cheek pouch (HCP) animal model was used to evaluate the ability of black raspberries to inhibit oral cavity tumors. Male Syrian Golden hamsters, 3-4 weeks of age, were fed 5% and 10% lyophilized black raspberries (LBR) in the diet for two weeks prior to treatment with a cancer inducing agent (i.e., 0.2% 7,12-dimethylbenz(a)anthracene in dimethylsulfoxide; hereafter DMBA) and for 10 weeks thereafter. The diets comprising 5% and 10% black raspberry extracts were prepared as described above and determined to comprise the components indicated in Table 1.

The cancer agent was applied to the oral cavities of the animals for eight weeks after which the animals were sacrificed 12-13 weeks from the beginning of DMBA treatment (Table 5) and the number and volume of tumors (mm³) was determined (Table 6). There was a significant difference (p=0.02) in the number of tumors observed between the 5% black raspberry extract and control groups (27 tumors/14 animals and 48 tumors/15 animals, respectively) and an intermediate number of tumors was observed in the 10% berry-treated animals (39 tumors/15 animals). These experiments show that dietary black raspberries will inhibit tumor formation in the oral cavity.

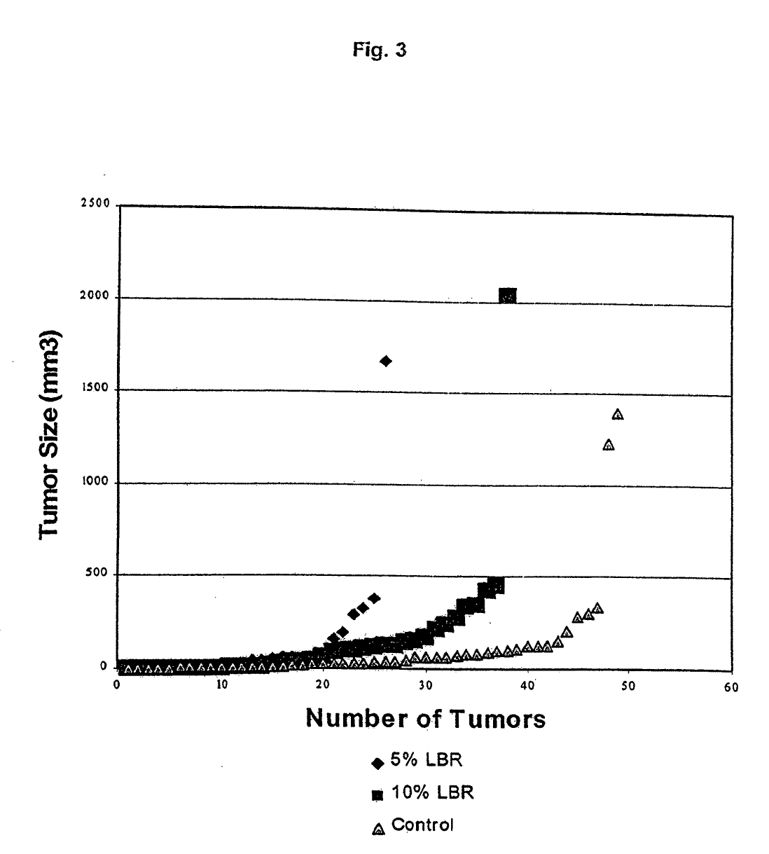

The inhibition of oral cancer, i.e., cheek pouch tumors both in size and numbers by lyophilized black raspberries is shown in FIG. 3. There was no difference in tumor size or incidence between the three groups at time of sacrifice. Except for one animal in the 5% extract supplement group all of the hamsters had at least one tumor arise in one or both pouches. However, only 9 of the 29 animals (31%) ingesting berries had 3 or more tumors compared with 10 of the 15 (67%) hamsters in the control group (p=0.03). Moreover, there were significantly fewer tumors per animal in the 5% LBR group (Table 6) when compared to the control group (27 tumors in 14 animals treated with 5% LBR and 48 tumors in 15 animals in the control group, p=0.02). The 15 animals treated with 10% lyophilized black raspberries had an intermediate number of tumors (39 tumors in 15 animals). No statistically significant differences in body weight or food consumption were observed between the control animals and animals given test diets comprising 5% or 10% lyophilized black raspberries.

TABLE 1
Carcinogen initiation and chemoprevention in vivo protocol*

| Group | Wk. 1-2 | Wk. 2-8 | Wk 9-10 | Wk. 12-13 |
|---|---|---|---|---|
| 1 | 5% LBR | DMBA | 5% LBR diet | Tumor |
| (N = 15) | | 3x/wk + | | harvest |
| | | 5% LBR diet | | |
| 2 | 10% LBR | DMBA | 10% LBR diet | Tumor |
| (N = 15) | | 3x/week + | | harvest |
| | | 10% LBR diet | | |
| 3 | Control diet | DMBA | Control diet | Tumor |
| (N = 15) | | 3x/week + | | harvest |
| | | control diet | | |

*DMBA in DMSO solvent was administered via cheek pouch painting for 8 weeks (3x/week) beginning 2 weeks after LBR diets were started. Hamsters were given 5% and 10% LBR diets during the 8 weeks of DMBA treatment and for two weeks following treatment. At 12-13 weeks, animals in the DMBA treated and control groups (Groups 1, 2 and 3) were sacrificed and cheek pouches analyzed for tumors, tumor size, and histopathologic changes.

TABLE 2
Inhibition of hamster cheek pouch tumors
by dietary consumption of LBR

| Group | | | #Tumors/ | Tumor |
|---|---|---|---|---|
| (N = 15) | Treatment^{a} | % Incidence^{b} | # Animals^{c} | Multiplicity |
| 1 | DMBA + | 93% | 27/14 | 1.93^{d} |
| | 5% LBR | (13/14) | | |
| 2 | DMBA + | 100% | 39/15 | 2.60 |
| | 10% LBR | (15/15) | | |
| 3 | DMBA | 100% | 48/15 | 3.20 |
| | control | (15/15) | | |

^{a}Hamsters were given LBR in the diet for 2 weeks, treated with DMBA and LBR for 8 weeks, and given LBR in the diet for 2 additional weeks.
^{b}Cumulative number of tumors in treated animals. One animal in Group 1 died of unknown cause.
^{c}Total tumors in both pouches. Nine of 29 animals in the berry groups had less than 3 tumors, whereas 10/15 animals in control group had more than 3 tumors (p = 0.03)
^{d}Significantly different than DMBA control (p = 0.02)

The following studies were performed to demonstrate the molecular mechanisms involved in the inhibition of carcinogenesis by a berry extract of the invention.

Accordingly, a black raspberry extract was investigated for its ability to modulate transactivation of the cell signaling intermediates AP-1 and NFκB as induced by benzo(a)pyrene diol-epoxide (BPDE), the resultant carcinogen of B(a)P, in mouse epidermal cells (i.e., JB-6 clone 41 cells).

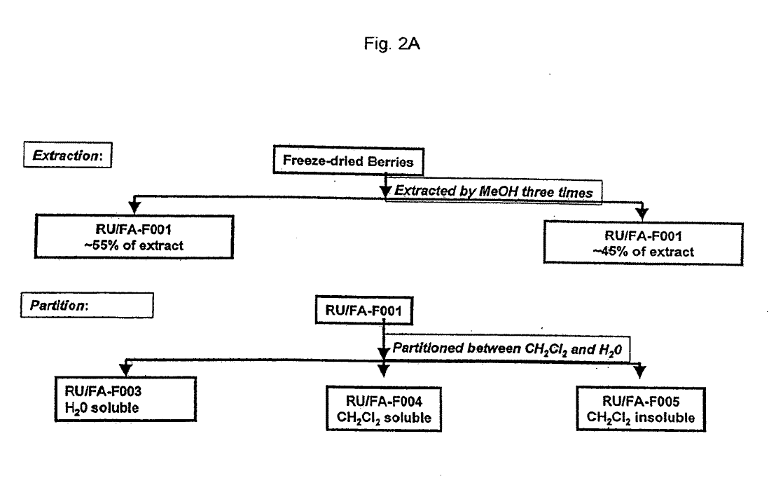

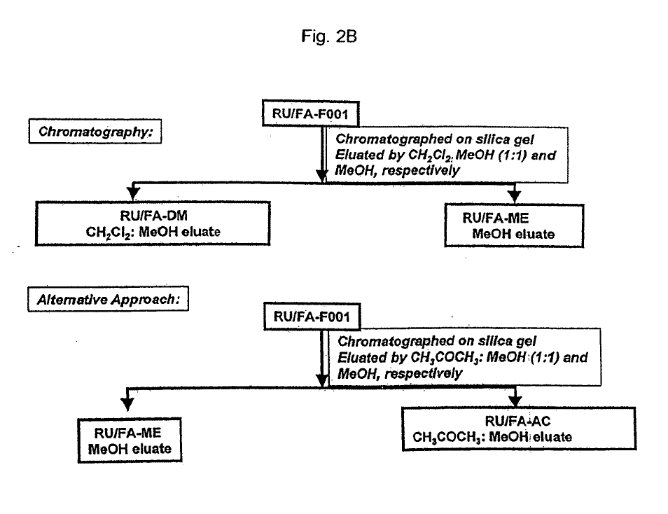

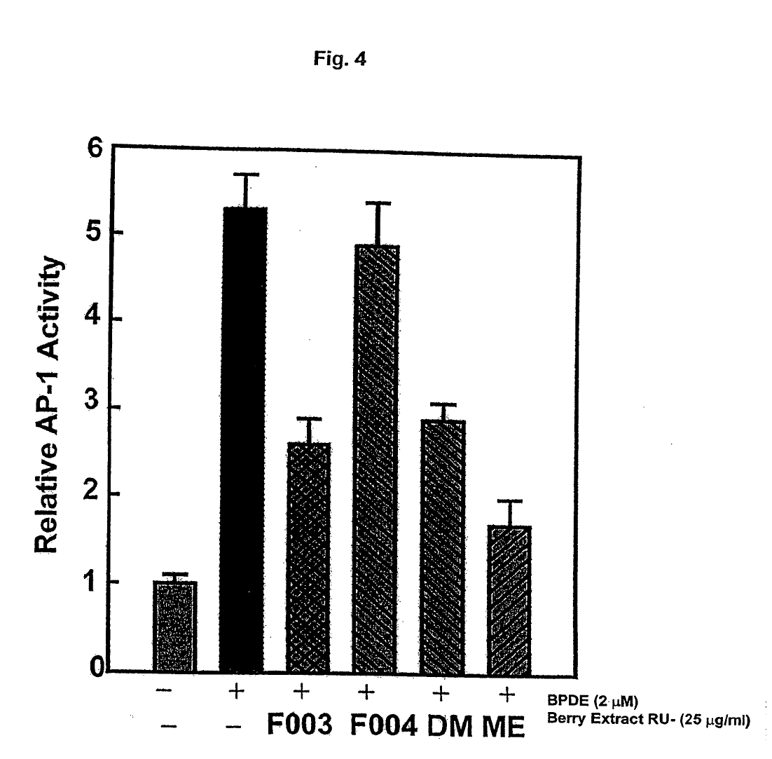

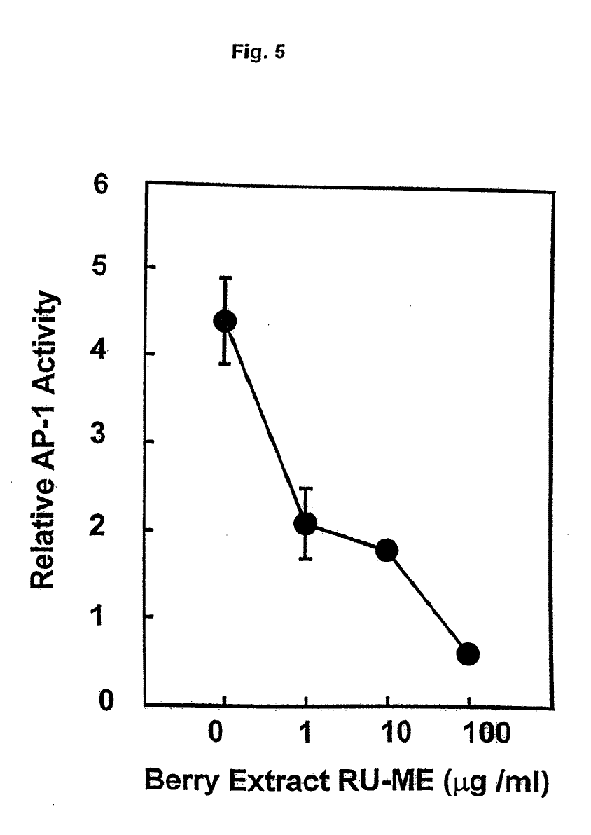

In particular, the potential effects of the black raspberry fractions (i.e., RU-F003, RU-F004, RU-DM, and RU-ME, see, e.g., FIGS. 2A, 2B) on BPDE-induced AP-1 activation on mouse P⁺1-1 cells were examined. Specifically, P⁺1-1 cells were pretreated with each of four fractions (RU-F003, RU-F004, RU-DM and RU-ME) at 25 μg/ml for 30 min, and then exposed to 2 μM BPDE to induce AP-1. Pretreatment of P⁺1-1 cells with either the RU-F003, RU-DM or RU-ME fractions resulted in a significant inhibition (P<0.05) of BPDE-induced AP-1 activity, while the RU-F004 extract had no effect (FIG. 4). The RU-ME fraction was the most potent inhibitor of AP-1 activity among the extracts tested, which is consistent with its potency as an inhibitor of B(a)P-induced cell transformation. The RU-ME fraction was inhibitory when added to the medium at only 1 μg/ml (FIG. 5). Note in FIG. 5, that the addition of 1 μg/ml of RU-ME to the medium resulted in an approximately 40% reduction of the measured activity of AP-1. Thus, these studies demonstrate that the therapeutic benefits of the fruit extracts co-elute with a fruit extract that is demonstrably soluble in an alcohol/water fraction as shown by these alcohol water extracts inhibiting AP-1 activity. While the specific compounds present in the fruit extract may also be present, possibly in a slightly different chemical form, in water and or alcohol/water insoluble residues of the fruit pulp, the particular biochemical activity of the therapeutically effective composition can be correlated with the ability of the alcohol/water fruit extract fraction to a reduction in the activity of cell signaling molecules such as AP-1.

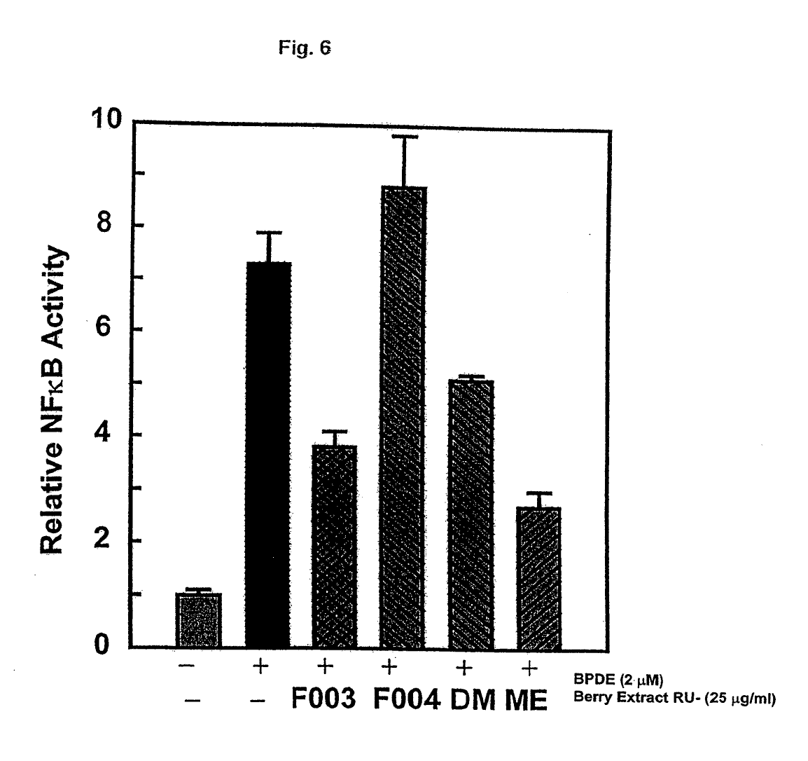

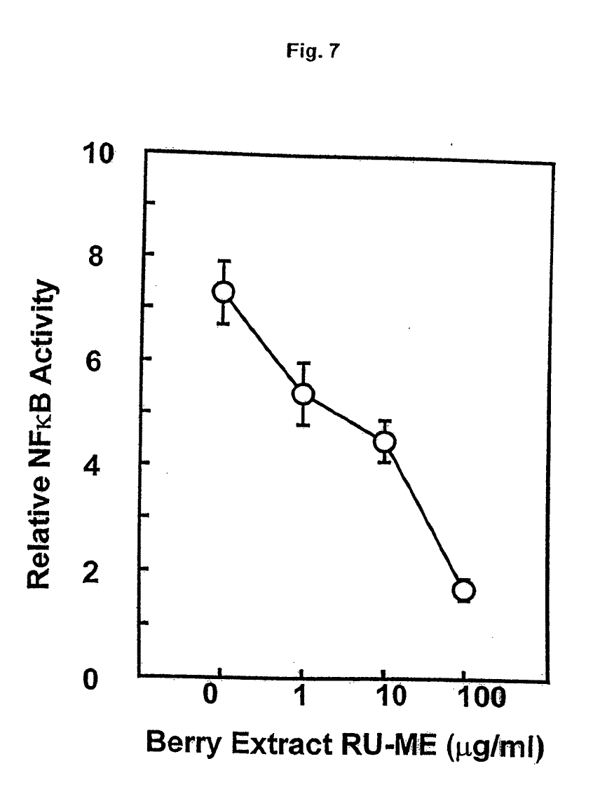

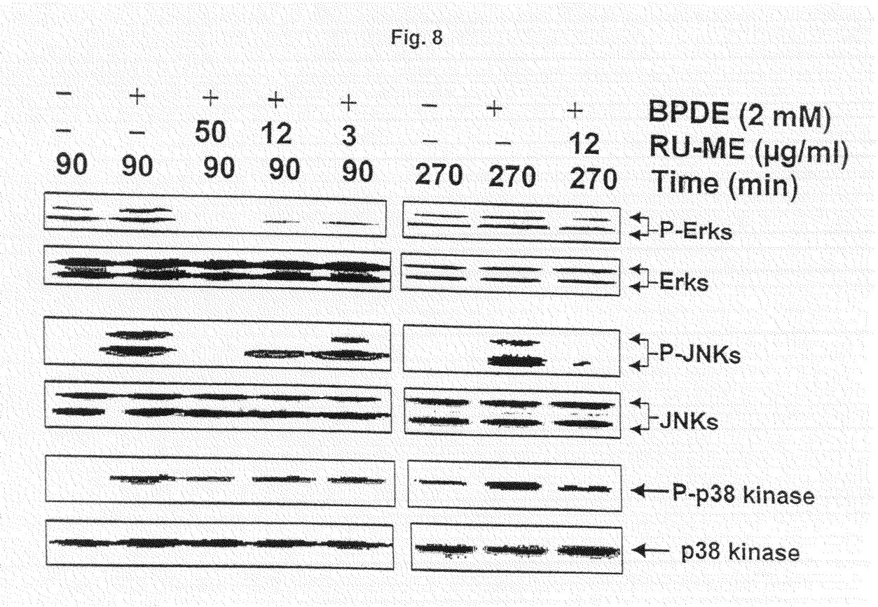

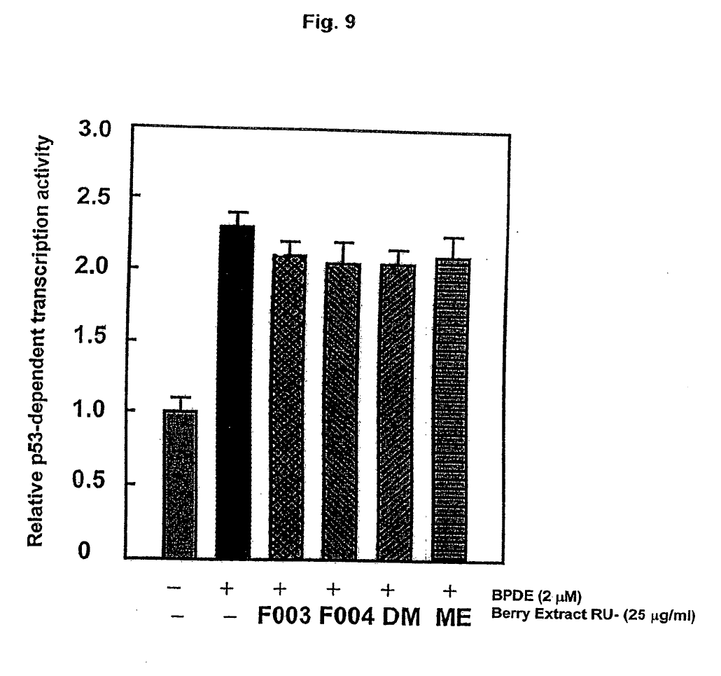

In another study, the effect of fruit extract fractions on the induction of NFκB by BPDE in mass1 cells, was examined. Specifically, pre-incubation of the cells with either the RU-F003, RU-DM or the RU-ME fraction led to a significant inhibition (P<0.05) of BPDE-induced NFκB activity in the cells (FIG. 6). In contrast, fraction RU-F004 did not inhibit NFκB activity. The RU-ME fraction was the most potent inhibitor of NFκB activity among the fractions tested. The inhibitory effect of RU-ME on BPDE-induced NFκB activity was observed to be in dose- and time-dependent manner (FIG. 7, FIG. 8). As seen in the studies above, BPDE-induced p53-dependent activation was not affected by any of the fractions tested on mass1 cells (FIG. 9).

To determine the conditions under which the berry fractions inhibit BPDE-induced activation of AP-1 and NFκB in CI 41 cells, the most active fraction, RU-ME, was added to cultured CI41 cells at different times before or after exposure of the cells to 2 μM BPDE. The inhibitory effect of the RU-ME fraction on both AP-1 and NFκB occurred only when RU-ME was added either before or along with the BPDE. RU-ME was not effective when added to the cells 3 hours after treatment with BPDE.

These data indicate that pre-treatment or simultaneous co-incubation of RU-ME with BPDE is required for inhibition of BPDE-induced activation of AP-1 and NFκB. Thus, these studies demonstrate that the therapeutic benefits of the fruit extracts co-elute with a fruit extract that is demonstrably soluble in an alcohol/water fraction as shown by these alcohol water extracts inhibiting AP-1 activity. While the specific compounds present in the fruit extract may also be present, possibly in a slightly different chemical form, in water and or alcohol/water insoluble residues of the fruit pulp, the particular biochemical activity of the therapeutically effective composition can be correlated with the ability of the alcohol/water fruit extract fraction to a reduction in the activity of cell signaling molecules such as AP-1. Moreover, it is shown by these studies that it is preferable to provide the disclosed fruit extract fraction prior to or near the initiation of active carcinomas. Thus, the alcohol/water fruit extract fraction provides either a chemopreventative benefit, by limiting the initiation of cancer, or limits the progression of initiated neoplasms to cancerous tumors.

Black raspberries contain multiple compounds with known chemopreventive activity. Among these, ellagic acid can react with BPDE to form covalently linked cis and trans adducts in which the reactive epoxide ring of the pyrene is open, rendering the BPDE harmless. In order to determine whether inhibition of BPDE-induced activation of AP-1 and NFκB by the RU-ME fraction might be due to a similar reaction of compounds in RU-ME with BPDE, the effect of RU-ME on BPDE-induced DNA adduct formation was tested. If compounds in RU-ME react with BPDE, then one might expect lowered levels of BPDE binding to CI 41 cell DNA. To determine the effect of RU-ME on BPDE-DNA adduct formation, cultured CI 41 cells were treated with [³H]-BPDE or [³H]-BPDE and RU-ME mixture. The ³H count in a known quantity of purified genomic DNA was determined. The number of BPDE-induced DNA adducts in a 10 kb genomic DNA fragment was then calculated. The results demonstrated that pre-incubation of the RU-ME fraction with BPDE did not reduce BPDE-DNA adduct formation in CI 41 cells. Accordingly, the mechanism of action of the extracts is not by the binding of extract components to BPDE, which would inhibit BPDE binding to cellular DNA.

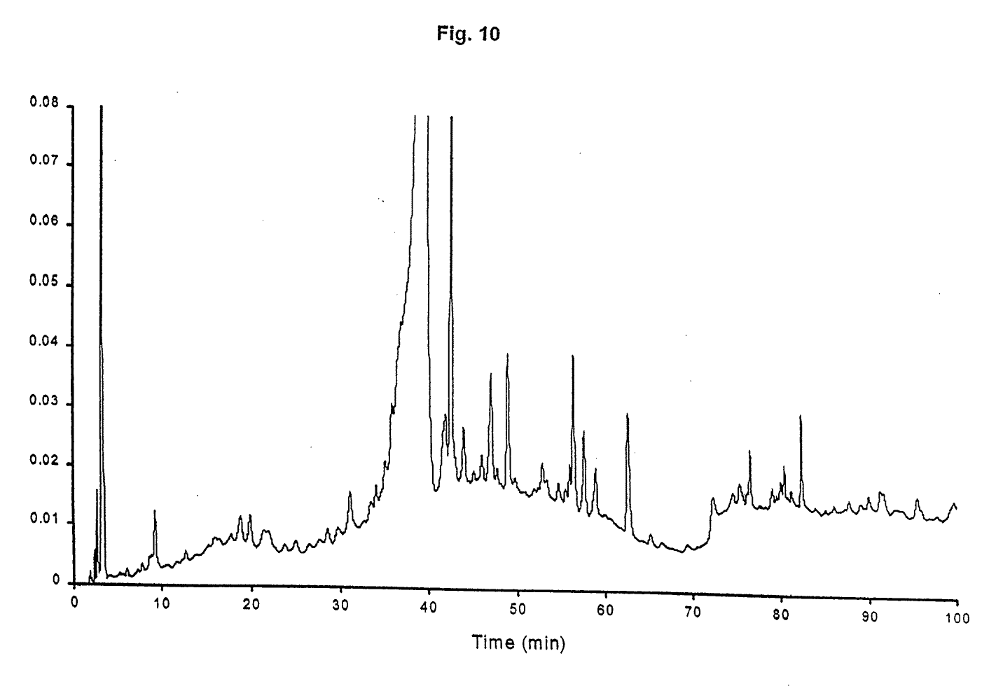

To test the effects of the RU-ME fraction on BPDE-induced activation of the ERKs, JNKs, and P38 kinases in CI 41 cells, the effects of RU-ME on phosphorylation of the MAP kinase family were tested. The results showed that pretreatment of cells with RU-ME led to a significant inhibition of phosphorylation of ERKs, JNKs and p38 kinase (FIG. 10), indicating that all three MAP kinase family members are involved in the inhibitory effect of RU-ME on AP-1 activation.

To determine whether inhibition of BPDE-induced NFκB by RU-ME is caused by inhibition of IκBα phosphorylation and degradation, IκBα phosphorylation in cells exposed to BPDE and RU-ME using phospho-specific antibody was determined. Results obtained indicate that pretreatment of cells with RU-ME inhibited BPDE-induced increase in phosphorylation of IκBα at 90 min, and degradation of IκBα protein at 270 min, after BPDE treatment.

Thus, the RU-ME fraction was determined to be the most potent inhibitor of BPDE-induced AP-1 and NFκB activities among the fractions tested, which is consistent with its potency as an inhibitor of B(a)P-induced cell transformation. In addition, the inhibitory effects of RU-ME on BPDE-induced activation of AP-1 and NFκB can be mediated via inhibition of MAP kinase activity and IκBα phosphorylation, respectively.

Accordingly, in view of the important roles of AP-1 and NFκB in tumor promotion, these results indicate that RU-ME is a major fraction for chemopreventive activity in black raspberry extracts, and that the anti-tumor progression activity of black raspberries can be mediated by impairing signal transduction pathways leading to activation of AP-1 and NFκB.

A preferred embodiment is modulation of undesired signal transduction activity. While chemopreventative benefit may be derived directly by the activity of the fruit extract fractions on by inhibiting (down-regulating) signal transduction components such as NF-Kβ, AP-1, Akt, iNOS, and or VEGF, as shown herein the fractions have anti-neoplastic activity that is closely correlated with the ability of those fractions to modulate signal transduction activity. Thus, while the therapeutic benefit may not arise solely from the modulation of said signal transduction components, it is apparent that those components are also useful as markers of the beneficial therapeutic activity of the fruit extract fractions. As is shown in the disclosure, including in the examples that follow, a two fold, five fold and even ten fold increase in the inhibitory activity against components of the signal transduction pathways is seen from use of the fruit extract fractions disclosed herein. Thus activity against AP-1, for instance, may be used either to standardize extracts, or as a marker for enhanced chemotherapeutic benefit.

Treatment of Other Diseases and Disorders

In yet another embodiment, the berry extracts and compositions derived therefrom (particularly those having an enhanced ability to modulate the regulation of cellular metabolism and or antioxidant activity) can be used in the treatment or prevention of a wide range of other diseases and disorders that include, respiratory, inflammatory, neurological, dermatological (e.g., actinic keratosis and dysplastic nevi of the skin, skin cancer), cardiovascular disease, stroke, inflammatory diseases (e.g., arthritis), as well as inhibiting aging. Indeed, a large volume of reported research provides evidence that disruption of the gene regulatory pathways involved in carcinogenesis also play a critical role in the above-mentioned conditions.

Accordingly, the berry extracts of the invention and compositions derived therefrom having both of these properties are especially well suited for the prevention and/or treatment of a broad spectrum of biological conditions. Moreover, such extracts and compositions of the invention also are well suited to the treatment of any yet to be characterized biological disorders or diseases that, at some level, are affected by or controlled by a mechanism associated with these properties.

In another embodiment, berry extracts of the invention and compositions derived therefrom (particularly those having high antioxidant activity) can be used to treat or prevent heart disease. Indeed, the efficacy of vitamin E (tocochromonals) in reducing cholesterol levels in animals, including humans, is well supported in the scientific literature.

Accordingly, the berry extracts of the invention, and compositions derived therefrom, can be used in the treatment of high cholesterol (cholesterolemia) and other associated conditions such as heart disease.

Hypercholesterolemic diseases and conditions that can be treated using the fruit extracts of the invention and compositions derived therefrom include, but are not limited to, atherosclerosis, arteriosclerosis, xanthomatosis, hyperlipoproteinemias, and familial and hypercholesterolemia. Hypercholesterolemic diseases are known to be affected by changes in NOS activity and NO levels. As evidence that certain embodiments disclosed herein can affect cholesterol levels, presumably through signaling interactions described herein, animals were fed an extract from black raspberry and their blood lipid compositions analyzed. Briefly, blood analyses of animals (i.e., laboratory rats) fed black raspberry extract added at 5% to their diets were conducted to determine the effects of the extract on blood lipid levels. Blood samples were collected at 33 weeks, placed in heparinized tubes and analyzed by Antech (Alsip, Ill.) using standard techniques. Importantly, black raspberry extract consumption at 5% of the diet significantly reduced blood cholesterol levels from 248.76±44.63 mg/dl in the diet control group to 223.57±44.81 mg/dl in the group administered the extract. The berry extract had no effect on other blood lipid values. This same method can be applied to determine the cholesterol lowering activity of the strawberry extracts of the invention. Thus, berry extracts (e.g., black raspberry) of the invention have significant cholesterol lowering potential as demonstrated using a relevant animal model.

Thrombotic diseases and conditions that may be treated using berry extracts of the invention and compositions derived therefrom include, but are not limited to, pulmonary disease (for example, involving reduced conductance, compliance, or constriction), excessive fluid accumulation or pulmonary edema, respiratory distress, asthma, pulmonary vascular permeability, pulmonary vasoconstriction, pulmonary hypertension, pulmonary embolism, cardiac ischemia, myocardial infarction, cardiopulmonary bypass associated dysfunction, vasoconstriction, organ dysfunction, platelet dysfunction, cardiac disease, chronic obstructive arterial disease caused by arteriosclerosis, vasoconstriction, renal artery stenosis, myocardial infarction, stroke, deep vein thrombosis, peripheral arterial occlusion, and other blood system thromboses.

Antiatherogenic diseases and conditions that can be treated using berry extracts of the invention and compositions derived therefrom include, but are not limited to, atherosclerosis, arteriosclerosis, myocardial infarction, ischemia (i.e., myocardial ischemia, brain ischemia, and renal ischemia) and strokes.

Inflammatory diseases and conditions that can be treated using berry extracts of the invention and compositions derived therefrom include, but are not limited to, essential hypertension, hypertension of congestive heart failure, renal dysfunction caused by reduced myocardia output, endotoxemia, chronic liver disease or hypertension, pulmonary inflammation in asthma, lung injury (bronchitis, pneumonia, or acute); rheumatic diseases (for example, rheumatoid arthritis or systemic lupus erythematosus), inflammatory bowel disease (for example, ulcerative colitis), irritable bowel disease (such as villous adenoma), gastrointestinal disorders caused by excess acids, pepsin or bile salts, skin diseases or trauma (such as burns or acid or caustic injury), rheumatoid diseases.

Immunoregulatory diseases and diseases that can be treated using berry extracts of the invention and compositions derived therefrom include, but are not limited to, autoimmune diseases, for example, AIDS, chronic fatigue syndrome, graft rejections, and other viral diseases that impair the immune system.

It is understood that the extracts of the invention (and fractions thereof) are capable of inhibiting any of the diseases or conditions described herein through the modulation, for example, via its antioxidant activity, of one or more mechanisms. Such mechanisms include, modulation of a chemical carcinogen prior to its metabolism or contact with a cell; modulation of the metabolism of a carcinogen, modulation of a carcinogen metabolite (e.g., by scavenging or binding to the metabolite before it can cause oxidative damage of a lipid, protein, or genetic material); and/or modulation of a cellular pathway (e.g., signal transduction or gene transcription).

Modulation of Cellular Metabolic Activity by Fruit Extracts

The invention is further embodied in the modulation of specific cellular metabolic activity by the extract compositions of the invention. As such, a method is provided through which to treat cell dysplasia, moderate the effects of neoplastic lesions and provide for a direct or adjunctive therapy for the treatment of cancer. The extracts of the invention are shown by the detailed data provided in the Examples that follow to possess the capability of directly or indirectly modulating the activity of specific enzymes, for instance, COX-2 and iNOS, altering the expression of angiogenic growth factors, for instance VEGF, and modulating the production or accumulation of signaling molecules such as nitric oxide, along with NFkB, AP-1 P13K/AKt, MAPK, HFAT-1, ERK Y2, P38 and their associated kinases. In the discourse that follows, the nature and effects of these beneficial activities of the extracts of the invention are further explained.

The invention is embodied in a dietary fruit powder, e.g., lyophilized black raspberry powder that significantly inhibits VEGF-C expression and inhibits angiogenesis in neoplastic lesions, as demonstrated by a reduction in microvessel formation when BRB powder is added to the diet of rats with NMBA-induced angiogenesis in the esophagus. In addition, the down-regulation of VEGF-C by BRB was significantly correlated to the modulation of COX-2 and iNOS by BRB. Those skilled in the art of oncology will recognize that angiogenesis plays a critical role in carcinogenesis. A reduction in the rate of angiogenesis in pre-neoplastic or neoplastic lesions is expected to positively correlate with a decrease in the rate of progression of such lesions to cancer and metastasis. In particular, VEGF-C is known to be expressed at elevated levels in human esophageal SCC. The ability of BRB extracts and compositions derived from those extracts are thus predicted to have beneficial anti-angiogenic potential for squamous cell carcinomas in humans, and for human esophageal SCC in particular. Because dietary BRB extracts are delivered directly to tissues associated with esophageal SCC, inclusion of BRB or other fruit extracts or compounds possessing the beneficial activity according to the invention in the diet is a preferred embodiment of the invention. Considering the deadly nature of esophageal SCC, and the rate expected for such carcinomas to progress to metastasis, the invention is further embodied in a method of providing therapy or prophylaxis to those patients diagnosed with esophageal SCC or at high risk for developing that or similar diseases.

It is recognized that vascularization of neoplastic tissues is greater for SCC than in the normal esophageal tissues (See Kitadai, et al., Clin. Cancer Res., 4: 2195-2200 (1998)). Esophageal carcinogenesis is considered a stepwise process with lesions progressing from normal cells to hyperplasia to dysplasia and then to carcinoma. The stage at which an “angiogenic switch” occurs, however, had not been previously defined. It has been generally accepted by artisans that angiogenesis is essential for tumor growth and metastases, which are dependent upon the acquisition of adequate oxygen and nutrient through blood supply. Several cytokines and growth factors are known to promote angiogenesis including transformation growth factor-β (TGF-β), transformation growth factor-α (TGF-α), platelet-derived growth factor, basic fibroblast growth factor and VEGF. VEGF is recognized as the most potent mitogen for vascular endothelial cells, and overexpression of VEGF has been strongly associated with the angiogenesis in many human cancers including esophageal SCC. It is also recognized that expression of a particular cytokines or growth factor can induce the expression of other factors.

The specific nature of the biological activity of the compositions of the invention is demonstrated by the effect of the composition to modulate the microvessel density (MVD) in the whole esophagus including in hyperplastic and dysplastic tissues, with hyperplastic and dysplastic tissue regions generally being considered to be considered as the precancerous lesions with at least the potential to develop into cancers. Fruits of the Roseaceae family and in particular black raspberries of the genus Rubus have many known compounds which have antioxidant and anti-inflammatory activities, including the flavonoids, ellagitannins, vitamins and phytosterols compounds identified herein. The nature of the biological activities of the specific chemical constituents are more fully delineated as part of the present disclosure.

The correlation between VEGF activity and COX-2 activity, or VEGF activity and iNOS activity disclosed herein is novel with respect to esophageal SCC. When rats were treated with NMBA alone, there was a positive correlation between VEGF activity and COX-2 activity, but not between VEGF/iNOS activities or COX-2/iNOS activities. When rats were treated with NMBA+BRB extract, however, there were correlations among all these three genes: VEGF/COX-2 activity, VEGF/iNOS activity and COX-2/iNOS activity. This novel observation indicates that the BRB extract composition is modulating cellular activity in a previously unrecognized manner.

The regulation of VEGF expression is a complex process wherein numerous genes, enzymes, signaling molecules and pathways are involved in an interconnected biochemical matrix, with components known to include Ras, COX-2 and iNOS. It has been demonstrated that certain Ras mutations contribute to tumor angiogenesis by enhancing the production of VEGF (see, e.g., Rak, et al., “Mutant ras oncogenes upregulate VEGF/VPF expression: implications for induction and inhibition of tumor angiogenesis.” Cancer Res., 55: 4575-4580 (1995)).

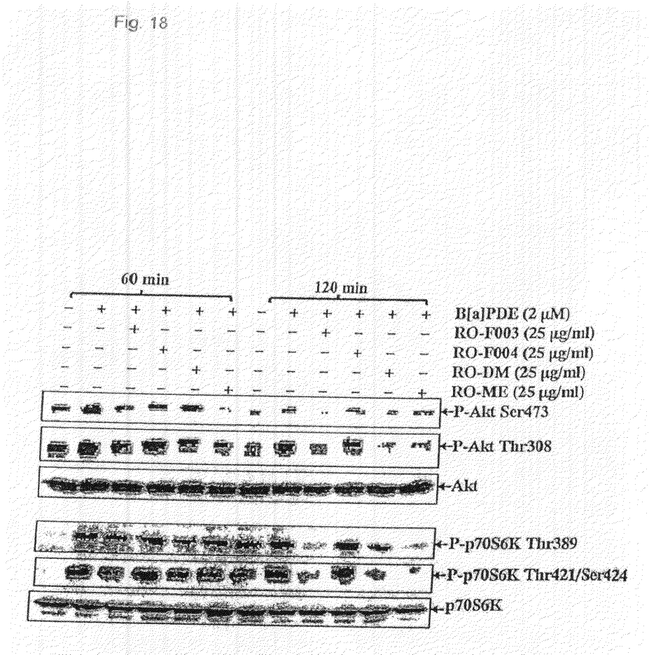

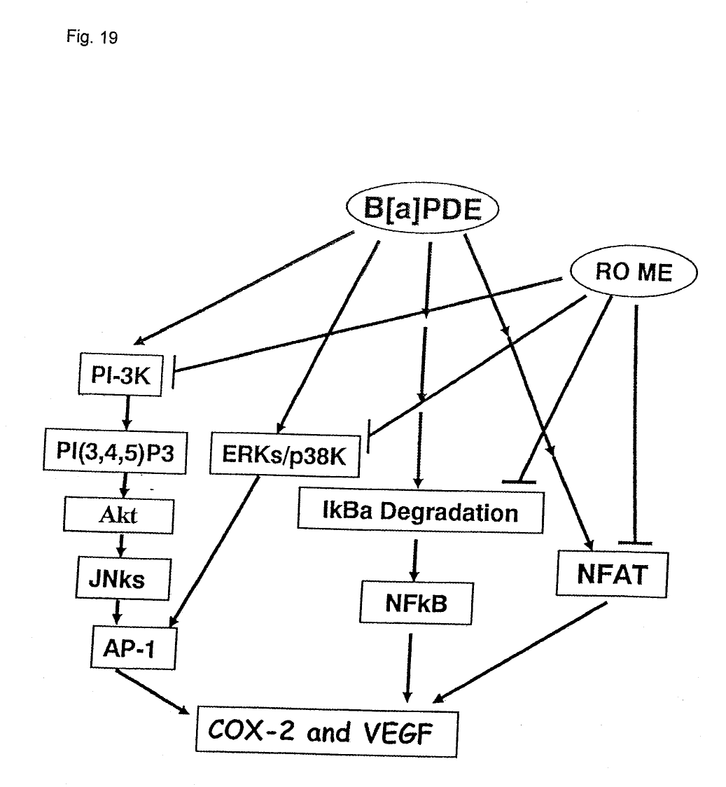

The precise mechanisms involved in the suppression of VEGF by BRB extracts and the suppression associated with the inhibition of COX-2 and iNOS by BRB extract, though incompletely understood, and are believed to be pleiotropic in nature, as shown in the diagram in FIG. 1. A putative explanation is that compounds from BRB extract inhibits VEGF expression through down-regulation of COX-2 and iNOS. COX-2 is an inducible enzyme, catalyzing the formation of prostanoids including prostaglandin E₂ (PGE₂) in response to certain stimuli such as growth factors, tumor promoters, hormones, and cytokines. The major contributions of COX-2 to carcinogenesis include enhancement of cell proliferation; increased resistance to apoptosis; and enhancement of angiogenesis. Overexpression of COX-2 occurs in various human cancers including esophageal SCC. The enhancement of angiogenesis by COX-2 occurs through stimulating interleukin-6 (IL-6), as modulated by PGE₂; inducing the up-regulation of the expression of VEGF by an increase of cAMP concentrations induced by binding of PGE₂ to the prostanoid receptor 2 (EP₂), thus leading to stimulation of the VEGF gene, and up-regulation of Bcl-2 expression that results in increased vascular endothelial cell survival. As an example, selective COX-2 inhibitors, such as NS-398 and JTE-522, are reported to effectively block angiogenesis by inhibiting VEGF expression. Furthermore, a positive correlation between COX-2 and VEGF has been previously reported in numerous human cancers including non-small cell lung cancer, colorectal cancer, head and neck squamous cell carcinoma, endometrial carcinoma, renal cell carcinoma, and ovarian cancer. The extensive roles of COX-2 in cancer development suggested that the therapeutic benefits of the extracts disclosed herein could modulate the activity of COX-2. Experiments demonstrate that B[a]PDE markedly induced COX-2 transcription and protein expression (FIGS. 18a and 18b), and that both RO-F003 and RO-ME fruit extract fractions strongly inhibited COX-2 expression. The RO-DM fraction, however, produced only a marginal effect, and the RO-F004 fraction had no effect, on COX-2 expression (FIG. 18a). Utilization of a range of different dose and time point studies demonstrates the inhibition dose range from 3.2-50 μg/ml of RO-ME as well as at all the time points observed (FIGS. 18b and 18c). Since COX-2 is a major target gene of AP-1, NFκB and NFAT, and black raspberry fraction RO-ME dramatically inhibits B[a]PDE-induced activation of all three transcription factors, it is reasonable to link RO-ME inhibition to reduction of COX-2 expression. Thus, the model biochemical pathway, as shown in FIG. 19 provides a schematic explanation of the molecular mechanisms that appear to be involved in providing the therapeutic benefits of the fruit extract fraction from Rubus, in that inhibition of COX-2 by Rubus extract is predicted to reduce VEGF expression, leading to a reduction of angiogenesis necessary for tumor expansion, and thus slowing the progression of oral cancer from dysplastic pre-cancerous cells to cancer and subsequent metastasis.

Similar in pattern to the activation of COX-2, iNOS is induced by certain cytokines, microbial products, and lipopolysaccharides to catalyze the formation of nitric oxide (NO) and citrulline from L-arginine. Increased NO production is associated with many disorders including cancer, for instance, through the reaction of NO with oxygen and super oxide, producing peroxynitrite with resulting DNA damage, and increased COX-2 expression. In addition, it is known that iNOS may indirectly promote tumor angiogenesis thorough the induction of COX-2 and induction of endothelial cell growth resulting from the elevation of NO level. Similar to the effect on COX-2, positive correlation between iNOS and VEGF activities has previously been reported in human cancers, such as colon, lung and gastric cancers. Thus, referring again to FIG. 1, BRB extract may directly or indirectly reduce VEGF expression or endothelial cell growth, slowing the progression of oral cancer. The therapeutic effects of BRB extracts may include an indirect down-regulation of VEGF through the inhibitory effect of BRB extracts on COX-2 and iNOS.

The compositions of the fruit extracts of the invention, including BRB extract, decrease the expression of COX-2 and iNOS as well as the level of their metabolites, PGE₂ and NO, in NMBA-treated rat esophagus. Indirect inhibition of VEGF and parallel inhibition of COX-2 and iNOS may operate through intermediate molecules upstream in the signal cascade through molecules controlling the expression of these genes, such as P13/Akt and nuclear factor-κB (NFκB) as modulated by BRB extract compositions. The phosphatidylinositol 3′-kinase (PI3K) signaling cascade plays a central role in regulating cell proliferation and survival by affecting the phosphorylation status of Akt, a downstream molecule in the PI3K cascade. The molecule pAkt is commonly used as the index for the activation of Akt. NFκB functions as a pivotal transcription factor to mediate the expression of many early response genes involved in carcinogenesis including COX-2 and iNOS (see, e.g., Barnes, P. J. and Karin, M., Nuclear factor-kB-a pivotal transcription factor in chronic inflammatory diseases. N. Engl. J. Med., 336: 1066-1071 (1997)). NFκB has two major subunits, p50 and p65. In normal cells, NFκB is sequestered in the cytoplasm in an inactive form through its association with its inhibitory protein, IκB-α. NFκB is activated by various signals, which include cytokines, mitogens, environmental and occupational particles, and bacterial products. The activation of NFκB may also controlled by the activation of Akt. Activation of NFκB results in a degradation of IκB-α by phosphorylation and translocation of NFκB to nucleus. The cellular levels of the molecules pp65 and p1κB-α both are commonly used as the index for the activation of NFκB.

In a further embodiment of the invention, BRB extracts modulated the expression of pAkt, pp65 and p1κB-α as determined by immunohistochemistry in normal esophagus and NMBA-treated esophagus. In normal rat esophagus, positive staining of pAkt and p1κB-α is only observed in macrophages not epithelial cells, and positive staining of pp65 is not been detected in normal esophagus. In contrast, in the esophagi treated with the tumorigen NMBA, extensive cytoplasmic staining of pAkt and p1κB-α and nucleus staining of pp65 in esophageal epithelium occurred, demonstrating an alteration of gene expression of these transactivating molecules. Surprisingly, by utilizing the BRB extract of the invention, the BRB extracts inhibited the activation of Akt and NFκB when rat tissues were treated with NMBA in vitro as above. Thus, the beneficial activity of fruit extracts, including BRB extracts, may provide a method to inhibit VEGF, COX-2 and iNOS through the suppression of upstream mediators, such as Akt and NFκB. Such beneficial activity may be distinct from a direct modulation of VEGF, COX-2 and iNOS.

To show the effects of the BRB extracts on the phosphorylation state of Akt, CI41 VEGF mass1 cells were seeded into each well of six-well plates, and cultured in 5% FBS-MEM at 37° C. After the cell density reached 70-80%, cells were first pretreated with black raspberry extract fractions (25 μg/ml) for 30 min, then exposed to B[a]PDE (2 μM) for 60 min or 120 min. Western blots were performed with either phosphospecific antibodies or non-phosphorylated antibodies against Akt and p70S6K. As shown in FIG. 18, the PI-3K/Akt pathway is involved in the inhibition of B[a]PDE-induced AP-1 activity. The pretreatment of CI 41 cells with Rubus occidentalis methanol extract (RO-ME) markedly inhibits B[a]PDE-induced PI-3K activation and phosphorylation of Akt at Thr308/Ser473 and p70S6K at Thr421/Ser424. Collectively, these and the other data disclosed herein indicate that Rubus fractions inhibit B[a]PDE-induced AP-1 activation through inhibiting activation of the PI-3K/Akt pathway.

The data included in the examples that follow clearly demonstrate the novel and unanticipated features of the invention that dietary fruit compositions, specifically BRB compositions, significantly inhibit VEGF-C expression and microvessel formation during tumorigenesis as demonstrated by the NMBA-induced rat esophageal tumorigenesis model. As angiogenesis is known to play a critical role in cancer progression, fruit compositions, specifically BRB compositions, are expected to provide an antiangiogenesis potential for utilization in a variety of therapies for hyperplasia, dysplasia, neoplasia and cancer. These beneficial activities associated with fruit compositions, specifically BRB compositions, are shown to include a beneficial correlation of the levels of VEGF, COX-2 and iNOS in the rats treated with dietary BRB. One possible mechanisms of these beneficial activities is that the modulation of angiogenesis by BRB is associated with an alteration of COX-2 and iNOS activity. Since existing anti-angiogenesis drugs have only modest effects derived by targeting only specific proteins, the fruit extract of the invention, fruit derived compositions, specifically BRB compositions, achieve promising beneficial effects by exhibiting pleiotropic effects on multiple pathways in cancer development. The invention is embodied in a beneficial activity that is different from existing antiangiogenic drugs in the nature of its broad effect on the mechanisms of angiogenesis. Thus, the administration of fruit powders, extracts and compositions derived from said substances, including raspberries, black raspberries, strawberries, and other related members of the Roseaceae family are expected to have beneficial applications in human.

Synergy with Other Components Derived From Berry Extracts and/or Exogenous Compounds

In another embodiment, berry extracts of the invention, or one or a combination of components derived therefrom, are administered to a subject with an additional (exogenous) compound, e.g., an anti-cancer agent such as a chemotherapeutic compound and/or in combination with, for example, radiotherapy for the treatment of cancer. Administration of berries or their fractions along with chemotherapeutic drugs can permit more long-term, low-dose treatment of cancer patients with chemotherapy. In addition, patients treated with radiotherapy can obtain some protection against the harmful effects of radiation on normal tissues since these effects can be attributed largely to oxidative damage.

Accordingly, the berry extracts of the invention and compositions derived therefrom (particularly those having antioxidant activity) can be used alone or in combination with chemotherapeutic agents (including radiotherapy) as potent anti-cancer agents.

Formulations and Methods of Administration

The berry extracts of the invention and compositions derived therefrom can be administered to a subject in any suitable form. For example, the extracts and compositions of the invention are sufficiently stable such that they can be readily prepared in a form suitable for adding to various foodstuffs including, for example, juice, fruit drinks, carbonated beverages, milk, nutritional drinks (e.g., Ensure™, Metracal™), ice cream, breakfast cereals, biscuits, cakes, muffins, cookies, toppings, bread, bagels, fiber bars, soups, crackers, baby formulae (e.g., Similac™), teas, salad dressings, cooking oils, and meat extenders. The berry extracts may also be delivered in the form of jellies, jams, or preserves.

In addition, berry extracts and compositions derived therefrom can be formulated as a pharmaceutical composition (e.g., a medicinal drug) for the treatment of specific disorders. In one embodiment, the fruit extract fraction is formulated into a salve for application to the skin in the prevention of skin cancer. In another embodiment, fruit extract fraction is formulated into a suppository that may be inserted into the rectum or vagina for treatment of dysplastic cells that may be present at those locations or be predicted to have a risk of being present at those locations. In another embodiment, berry extracts of the invention and compositions derived therefrom can be formulated as a dietary supplement. Suitable additives, carriers and methods for preparing such formulations are well known in the art.

One advantage of utilizing the organic solvent fractions described herein over simply adding freeze dried fruit to the diets of patients who could benefit from the pharmaceutically active components of the fruit extract fraction is a reduction in the quantity of free sugars that are present in unprocessed fruit pulp. In particular, free sugars such as fructose and sucrose are present in relatively high concentrations in unprocessed fruit. Thus, although patients could consume large quantities of black raspberry, for instance, and derive a benefit, not only would it be difficult to consume sufficient quantities of fruit to provide a maximal benefit, but also there would be substantial expense, and a substantial caloric load associated with high fruit consumption. For example, in a study of patients consuming a daily quantity of freeze dried raspberries believed to be sufficient to provide some benefit, patients routinely experienced a weight gain during the six month study, in some cases a substantial weight gain. The additional calories consumed as freeze dried berries contributed an additional 100 to 150 kcal per day to the diet. By extracting only those most beneficial components of the fruit extract fraction, and providing that composition to patients, most of the additional sugars and calories are removed, while making consumption of a therapeutically effective amount more practicable.

For example, pharmaceutical compositions may take the form of tablets, capsules, emulsions, suspensions and powders for oral administration, sterile solutions or emulsions for parenteral administration, sterile solutions for intravenous administration and gels, lotions and cremes for topical application, and suppositories for colorectal or cervical administration. The pharmaceutical compositions may be administered to humans and animals in a safe and pharmaceutically effective amount to elicit any of the desired results indicated for the compounds and mixtures described herein. In addition, the extracts of the invention may be used in cosmetics.

The pharmaceutical compositions of this invention typically comprise a pharmaceutically effective amount of a berry extract or fraction thereof containing, for example, a berry extract with antioxidant activity, and, if suitable, a pharmaceutically acceptable carrier. Such carriers may be solid or liquid, such as, for example, cornstarch, lactose, sucrose, olive oil, or sesame oil. If a solid carrier is used, the dosage forms may be tablets, capsules or lozenges. Liquid dosage forms include soft gelatin capsules, syrup or liquid suspension.

Therapeutic and prophylactic methods of this invention comprise the step of treating patients or animals in a pharmaceutically acceptable manner with the compositions and mixtures described herein.

The pharmaceutical compositions of this invention may be employed in a conventional manner for the treatment and prevention of any of the aforementioned diseases and conditions. Such methods of treatment and prophylaxis are well-recognized in the art and may be chosen by those of ordinary skill in the art from the available methods and techniques. Generally, dosage ranges may be from about 1 to about 1000 mg/day. However, lower or higher dosages may be employed. The specific dosage and treatment regimens selected will depend upon factors such as the patient's or animal's health, and the severity and course of the patient's (or animal's) condition and the judgment of the treating physician. In certain embodiments, a diet is formulated to include the freeze dried berry powders of the invention in a concentration from about 1% to about 20% by weight. In a preferred embodiment, the concentration is about 5% by weight. In another preferred embodiment, the concentration is about 10%. In yet another preferred embodiment, the concentration is about 15%. In still another embodiment, the concentration is about 20%.

The berry extracts of the invention and compositions derived therefrom also can be used in combination with conventional therapeutics used in the treatment or prophylaxis of any of the aforementioned diseases. Such combination therapies advantageously utilize lower dosages of those conventional therapeutics, thus avoiding possible toxicity incurred when those agents are used alone. For example, other nutrients or medications, for example, cholesterol lowering drugs, chemotherapeutic agents, and/or radiotherapy.

In foodstuffs, the berry extracts of the invention and compositions derived therefrom can be used with any suitable carrier or edible additive. For example, the berry extracts of the invention may be used in foodstuffs, such as baked goods (for example, breads, muffins, and pastries), and cereals. The berry extracts and compositions derived therefrom also can be emulsified and used in a variety of water-based foodstuffs, such as drinks, for example, juice drinks, sports drinks, and drink mixes. Advantageously, the above-mentioned foodstuffs may be included in low fat, low cholesterol, or otherwise restricted dietary regimens.

Pharmaceutical compositions, dietary supplements, and foodstuffs of the present invention can be administered to humans and animals such as, for example, livestock and poultry.

This invention is further illustrated by the following examples which should not be construed as limiting.

### EXAMPLES

### Example 1

### Definitions

The term “analog” as in “a compound or analog thereof”, is intended to include compounds that are structurally similar but not identical to the compound, but retain some or all of the anti-cancer properties of the compound.

As used herein the term “anti-cancer activity” or “anti-cancer properties” refers to the inhibition (in part or in whole) or prevention of a cancer as defined herein. Anti-cancer activity includes, e.g., the ability to reduce, prevent, or repair genetic damage, modulate undesired cell proliferation, modulate misregulated cell death, or modulate mechanisms of metastasis (e.g., ability to migrate).

The term “antioxidants” includes chemical compounds that can absorb an oxygen radical, e.g., ascorbic acid and phenolic compounds. The term “antioxidant activity” refers to a measurable level of oxygen radical scavenging activity, e.g., the oxygen radical absorbance capacity (ORAC) of an extract, fraction, or compound. The term “antioxidant responsive condition” includes any disease or condition that is associated with the presence of undesired oxidation, oxygen radicals, or other free radicals.

The term “berry” is intended to mean a succulent fleshy fruit in which the seeds are embedded in the pulp, such as, for instance, grape, cranberry, and blueberry; aggregate fruits with external seeds such as a strawberry (e.g., strawberries of the genus Fragaria, e.g., Fragaria ananassa), and aggregate fruits containing clustered berries (each with one or more seeds) such as a raspberry (e.g., a red or black raspberry, e.g., raspberries of the genus Rubus, e.g., Rubus occidentalis and Rubus urinus) and mulberry. The term berry as used herein also includes fruits which are not botanically considered berries, but which are commonly considered by consumers to be berries.

The plant taxonomic family Roseaceae contains many different genetically related plant species. With respect to the genus Rubus, while there are many species within the genus, those species are taxonomically very closely related. The members of the genus Rubus are predominantly characterized as caneberries, and although there are many species of caneberries, certain of either wild or domesticated species are known to artisans produce fleshy fruits which are harvestable as a food source. Other caneberries have not been selected to produce a large fleshy fruit, yet biochemically and taxonomically share many features. See also Wada, et al., J. Agric. Food Chem. (2002).

The term “berry extract” includes a berry extract isolated from its natural context (i.e., the fruit), e.g., concentrated freeze-dried berries (e.g., lyophilized). Preferably, “isolated berry extract” of the invention is enriched for the presence of increased antioxidant activity, for example, has a high oxygen radical absorbance capacity (ORAC) (e.g., a value at least about 5.0 per mg, and preferably, between at least about 5-10, more preferably, between at least about 10-15, most preferably, at least about 15 or greater), has increased levels of antioxidants, has a high vitamin content (e.g., vitamin A, vitamin E (tocochromonal) content, vitamin C (ascorbic acid), folic acid, other desirable components (e.g., carotenoids, phenolic compounds, phytosterols, and minerals), and is substantially free of undesired impurities, e.g., stems.

The term fruit extract refers to fruits which have been transformed in some manner, for example, pureed, freeze-dried and particularly by modifications resulting from freezing and dehydration resulting in a freeze-dried extract enriched for antioxidant activity and other beneficial compounds.” In general a fruit extract is defined to include a mixture of a wide variety of compounds from the originating fruit.

The term “fraction” refers to a composition that has been separated into pools of substituent components of the fractionated composition, with such fractionation being performed by a variety of means, including, but not limited to density, solubility, mobility and chromatographic methods. Further separation of a fraction by alternative means of fractionation may yield subfractions. The term “berry extract fraction” includes a berry extract that has been fractionated with a solvent and is, preferably substantially free of solvent (e.g., at least 80-90%, preferably 90-99%, more preferably greater than 99%, and most preferably greater than 99.7%) as determined by standard techniques (e.g., gas chromatography), and/or off-flavors (as determined by taste and smell).

The term “cancer” or “malignancy” are used interchangeably and include any neoplasm (e.g., benign or malignant), such as a carcinoma (i.e., usually derived from epithelial cells, e.g., skin cancer, and aerodigestive tract cancer, such as an oral, esophageal, or colon cancer) or sarcoma (usually derived from connective tissue cells, e.g., a bone or muscle cancer) or a cancer of the blood, such as a erythroleukemia (a red blood cell cancer) or leukemia (a white blood cell cancer). A “malignant” cancer (i.e., a malignancy) can also be metastatic, i.e., have acquired the ability to transfer from one organ or tissue to another not directly connected, e.g., through the blood stream or lymphatic system.

The term “cardiovascular disease” includes, for example, hypercholesterolemia, thrombotic disease, and artherogenic disease.

The term “anti-hypercholesterolemic activity” and “cholesterol lowering activity” refers to the ability to regulate cholesterol metabolism or reduce serum cholesterol levels in a subject. The term “hypercholesterolemia” refers to abnormally high serum levels of cholesterol, typically due to defective cholesterol metabolism in a subject or diet.

The term “carotenoid” includes, for example, α-carotene, β-carotene, zeaxanthin, and leutin.

The term “dietary supplement” includes a compound or composition used to supplement the diet of an animal or human.

The term “exogenous” means the component is derived or obtained from a source other than fruit. Exogenous compounds suitable for adding to a fruit or berry extract of the invention (or fractions thereof) include, for example, one or more pharmaceuticals, chemotherapeutic agents, and/or radiotherapy.

The term “foodstuff” includes any edible substance that can be used as or in food for an animal or human. Foodstuffs also include substances that may be used in the preparation of foods such as cooking oils or food additives. Foodstuffs also include dietary supplements designed to, e.g., supplement the diet of an animal or human.

The terms “health promoting”, “therapeutic” and “therapeutically effective” are used interchangeably herein, and refer to the prevention or treatment of a disease or condition in a human or other animal, or to the maintenance of good health in a human or other animal, resulting from the administration of a berry extract (or fraction thereof) of the invention, or a composition derived therefrom. Such health benefits can include, for example, nutritional, physiological, mental, and neurological health benefits.

The term “isolated” refers to the removal or change of a composition or compound from its natural context, e.g., the berry.

The term “mineral” includes, e.g., any mineral that is naturally present at some measurable level in the berry extracts (or fractions thereof) and includes, e.g., calcium, magnesium, potassium, zinc, and selenium.

The term “native” refers to the originating berry source.

The term “pharmaceutical composition” or “therapeutic composition” refers to a composition formulated for therapeutic use and may further comprise, e.g., a pharmaceutically acceptable carrier. The term “pharmaceutically effective amount” refers to an amount effective to achieve a desired therapeutic effect, such as lowering tumor incidence, metastasis, undesired lipid levels in the blood, preventing thrombosis, preventing or treating inflammatory diseases, reducing serum biomarkers of inflammation, immunoregulatory diseases, fever, edema, cancer, or signs of aging.

The phrase “prevention of disease” relates to the use of the invention to reduce the frequency, severity, or duration (of disease) or as a prophylactic measure to reduce the onset or incidence of disease.

The term “phenolic compound” includes a compound that has an aromatic acid having one or more hydroxyl groups on the benzene ring and is naturally present at some measurable level in the berry extract (or fraction thereof) and includes, for example, ellagic acid, ferulic acid, anthocyanin, including cyanidin and pelargonidin, quercetin, kaempferol, and analogs thereof.

The term “physically disrupting” includes any appropriate physical manipulation (e.g., by mechanical means, e.g., using a masher, juicer, pulper, or, e.g., by sonication) that breaks (e.g., decharacterizes) the fruit into, e.g., skin, seeds and juice, e.g., into a puree.

The term “phytosterol” includes any sterol e.g., that is naturally present at some measurable level in the berry extracts (or fractions thereof) and includes, for example, β-sitosterol, campesterol, stigmasterol, and analogs thereof.

The term “vitamin” includes, for example, vitamin A, vitamin E, vitamin C, folic acid, but also any other art recognized vitamins. The term “vitamin A” generally includes retinal, retinol, retinoic acid, or a combination thereof. The term “vitamin C” generally refers to ascorbic acid. The term “vitamin E” generally includes tocochromanol compounds such as tocopherol or tocotrienol compounds.

### Example 2

### Materials and Methods

Throughout the examples, the following materials and methods were used unless otherwise stated.

In general, the practice of the present invention employs, unless otherwise indicated, conventional techniques of chemistry, e.g., food chemistry. Other techniques for carrying out the invention, for example, for preparing fruit extracts (and fractions thereof) and performing animal or cell-based assays for determining the anti-cancer properties of an extract (or fractions thereof), can be found, for example, in: Carlton et al., Carcinogenesis, 22:441-446 (2001); Stoner et al., Tox. Sciences Supp. 95-100 (1999); Carlton et al., Cancer Letters, 159:113-117 (2000); Xue et al., Carcinogenesis, 22:351-356 (2001); Harris et al., Proc. Amer. Assoc. Can. Res., pg 177 (Abstract) (2001); Xue et al., Tox. Sciences Supp., 54:267 (Abstract) (2000); Kresty et al., Proc. Amer. Assoc. Can. Res., 40:59 (Abstract) (1999); Kresty et al., Proc. Amer. Assoc. Can. Res., 39:18 (Abstract) (1998); Stoner et al., Proc. Amer. Assoc. Can. Res., 38:367 (Abstract) (1997); Kresty, et al., Can. Res. 61:6112-6119 (2001); Huang et al. Proc. Natl. Acad. Sci. USA. 95:156-161, (1998), and Casto et al., Anticancer Research, 22:4005 (2002).

Preparation of Extracts and Fractions Thereof

Preparation of extracts by freeze-drying was carried out as described herein using standard techniques. Techniques for solvent extraction of desirable fractions of the extracts (referred to as extract fractions) were carried out as described herein and as diagrammed in the figures of the application. The alcohol fraction isolated using methanol (ME) is also isolated using ethanol (Et), including ethanol/H20 at 80:20, in some studies. The process of obtaining the alcohol extracts are diagrammed in FIG. 2 and FIG. 3 of the application. Ethanol is a preferred extraction vehicle for extracts intended for human administration.

Analysis of Extract/Fraction Antioxidant Activity

Antioxidant activity was typically measured as a function of the oxygen radical absorbance capacity (ORAC) of the sample which was determined using standard techniques. ORAC is defined as “a scalar value useful for comparing the antioxidant content of different foods or nutritional supplements” and is a method of measuring antioxidant capacities of foods developed at the National Institute on Aging. See also, for instance, Huang, D., et al., “Development and validation of oxygen radical absorbance capacity assay for lipophilic antioxidants using randomly methylated cyclodextrin as the solubility enhancer.” J. Agric. Food

Chem. 50: 1815-1821 (2002); and Prior R. L. and Cao, G. H., “Analysis of botanicals and dietary supplements for antioxidant capacity: A review.” J. AOAC Int. 83 (4): 950-956 (2000). Briefly, in the ORAC assay mixture, B-PE was used as a target of free radical change, AAPH as a peroxyl radical generator, and Trolox as a control standard, and fluorescence (at, e.g., the following wavelengths of 540 nm (excitation) and 565 nm (emission)), was measured after addition of AAPH to the extract or fraction (e.g., as described in Wang et al., J. Agric. Food Chem., 44:701-705 (1996); and Cao et al., J. Nutr. 128:2383-90 (1998)).

For measuring the antioxidant activity of an extract of the invention (or fraction thereof) in altering the levels of particular radicals, electron spin resonance (ESR) spin trapping measurements can be made using standard techniques (e.g., as described in Leonard et al., J. of Environ. Path., Tox., and One. 19:49-60 (2000)).

Analysis of Extract/Fraction Components

Typically, compounds from the berry extracts are analyzed using art-recognized techniques such as solvent extraction and HPLC analysis. Components of the extracts, for example, carotenoids, phenolic compounds, phytosterols can be extracted and analyzed using, for example, thin layer chromatography and high-performance liquid chromatography. For example, the material can be fractionated on thin-layer chromatography (TLC) plates where the individual bands that are subsequently resolved can be scraped and extracted with a chloroform/methanol solvent. These resultant samples can then be analyzed using, e.g., gas and high-performance liquid chromatography (HPLC).

Other methods known in the art may also be employed, in place of or in combination with, the methods described above for isolating berry extract components, particularly to “scale up” the quantity of the isolated components. For example, chromatographic techniques may be used for isolating components of the berry extracts of the invention, in sufficient and pure quantities, such that the component may be administered alone or as part of a composition or product described herein (e.g., foodstuffs, dietary supplements, pharmaceuticals, etc.).

In particular, gas liquid chromatography, gas solid chromatography, high pressure or high performance liquid chromatography (HPLC) (e.g., normal, reverse, or chiral), ion exchange chromatography, or size exclusion chromatography can be employed as described, for example, in Advances in Chromatography, Brown, Eds., Marcel Dekker, Pub. (1998); Basic Gas Chromatography, Harold et al., John Wiley & Sons, Pub. (1997); Column Handbook for Size Exclusion Chromatography, Wu, Ed., Academic Press, Pub. (1999); Fundamentals of Preparative and Nonlinear Chromatography, Guichon et al., Eds., Academic Press, Pub. (1994); Handbook of Process Chromatography: A Guide to Optimization, Scale-Up and Validation, Hagel et al., Eds., Academic Press, Pub. (1997); HPLC Methods for Pharmaceutical Analysis, Lunn et al., John Wiley & Sons, Pub. (1997); Practical High-Performance Liquid Chromatography, Meyer, Wiley-Liss, Pub. (1999); and Hecht, et al., Carcinogenesis (2006), each of which is incorporated by reference herein.

Animal Diet Preparation

Animal assays were carried out using art-recognized techniques as described in the examples, and for example, as described in: Carlton et al., Carcinogenesis, 22:441-446 (2001) and Carlton et al., Cancer Letters, 159:113-117 (2000). All of the experimental conditions were in accordance with NIH Guidelines and with protocols approved by The Ohio State University Animal Care and Use Committee.

For most studies, black raspberries (Jewel variety) were supplied by the Dale Stokes Berry Farm (Wilmington, Ohio) and shipped frozen to Van Drunen Farms (Momence Ill.) for freeze-drying. The gross composition of the lyophilized black raspberry composition was determined by Covance Laboratories (Madison, Wis.) as presented in Table 3, along with the composition of two other lyophilized black raspberry (LBR) extracts used in dietary and other studies. The LBR powder was mixed into a modified AIN-76A diet at 5% and 10% concentrations with the concentration of cornstarch adjusted to maintain an isocaloric diet among all experimental groups. Berry-containing and control diets were prepared every two weeks, 140-170 grams measured into pint rat feeding jars, and stored at 4° C. Two jars were placed into each cage, feeding jars were rotated with each jar being replaced every 5-8 days with fresh feed, and the before and after weights of the jars recorded.

Male Syrian Golden hamsters (Mesocricetus auratus), 3-4 weeks of age, were obtained from the Charles River Laboratories (Wilmington, Mass.). Three animals each were placed in plastic bottom cages with hardwood chip bedding and allowed to acclimate for one week. Food (AIN-76A, a modified semi-synthetic, high starch diet, Dyets Inc., Bethlehem, Pa.) and water were given ad libitum with the AIN-76A powdered diet provided in rat feeding jars. Animals were weighed weekly during berry extract and carcinogen treatment.

Male Fisher 344 rats, 4-5 weeks old, were obtained from Harlan Sprague Dawley (Indianapolis, Ind.). The animals were housed 3 per cage under standard conditions (20±2° C.; 50±10% relative humidity; 12 hour light/dark cycles). Typically, food and water were available ad libitum, and hygienic conditions were maintained by twice weekly cage changes and routine cleaning of the animal rooms. Beginning 2 weeks after acclimation to the animal facility, the rats were placed on a modified AIN-76A synthetic diet (Dyets Inc., Bethlehem, Pa.) containing 20% casein, 0.3% D, L-methionine, 52% cornstarch, 13% dextrose, 5% cellulose, 5% corn oil, 3.5% American Institute of Nutrition salt mixture, 1% American Institute of Nutrition vitamin mixture, and 0.2% choline bitartrate (Dyets, Inc., Bethlehem, Pa.). The diet was routinely stored at 4° C. prior to preparation of experimental diets.

Experimental diets containing 5% and 10% Black raspberry (BRB) were prepared fresh weekly and stored at 4° C. Berry powder was mixed into AIN-76A diet (modified by reducing the concentration of cornstarch by 5% to maintain an isocaloric diet) for 25 minutes with a Hobart mixer (Troy, Ohio). Fresh experimental and control diets were placed in glass feeding jars weekly.

Chemicals and Reagent Kits.

The agent azoxymethane was obtained from Sigma Chemical Co., were purified by HPLC to greater than 98% purity. The agent 7,12-dimethylbenz(a)anthracene (DMBA) was obtained from Sigma-Aldrich (Milwaukee, Wis.) and dissolved at an 0.2% concentration in dimethylsulfoxide (DMSO). DMSO was obtained from Fisher Scientific, Pittsburgh, Pa. or from Sigma Chemical Company (St. Louis, Mo.); 4-(methylnitrosamino)-1-(3-pyridyl)-1-butanone (NNK) was obtained from Toronto Research Chemicals, Ontario, Canada and benzo(a)pyrene (BaP) from Sigma Chemical Co., St. Louis, Mo. The BaP/NNK mixture was prepared at a 1% concentration in DMSO. NMBA was obtained from Ash Stevens (Detroit, Mich.) and determined to be 98% pure by high-performance liquid chromatography. PBIT and the Nitrate/Nitrite Colorimetric Assay kits were obtained from Cayman Chemical Company (Ann Arbor, Mich.). The QuantiTect SYBR Green reverse transcription-PCR(RT-PCR) kit was purchased from Qiagen Inc. (Valencia, Calif.). The Prostaglandin E2 Biotrak Enzyme Immunoassay System was purchased from Amersham Biosciences, Corp. (Piscataway, N.J.). CD34 antibody was obtained from BioGenex, Inc. (San Ramon, Calif.).

Induction of Tumors and Chemoprevention Protocol for Animal Models

Tumors were induced in animal models, as specifically described, or in a manner similar to the methods that follow. For instance, groups of 15 hamsters, 3-4 weeks of age, were treated with carcinogen according to a modification of the initial methods as described (Morris et al., J. Dental Res. 40:3-15 (1961)). Hamsters were lightly anesthetized with Isoflurane and the opening of the pouch made accessible by inserting a small metal pegboard hook at the side of the mouth and gently pulling the hook laterally away from the hamster to expose the interior surface of the pouch. Animals were treated by painting both surfaces of each pouch 3 times weekly for 8 weeks with an 0.2% solution of DMBA dissolved in DMSO using a No. 4 camel hair brush (Dachi et al., Cancer Res. 27:1183-1185 (1967)). Tumors of sufficient mass in the control groups (3-10 mm in greatest length) suitable for final analyses appeared in 70-77 days (10 to 11 weeks) after beginning DMBA treatment. Twelve to thirteen weeks from the beginning of berry treatment and following CO₂ euthanasia, tumors were harvested, processed for evaluation, and final histologic examination performed after fixing and staining. Hematoxylin and eosin stained hamster cheek pouches were evaluated and histologically characterized by a board-certified oral pathologist in the College of Dentistry at The Ohio State University.

³²P-Postlabeling Assays for DMBA Adduct Analysis from Animal Model Tissues

Assays for determining DMBA induced adduct formation were typically carried out as follows. Two groups of six hamsters each were treated with a 5% concentration of black raspberry extract in a modified AIN-76A diet for two weeks. One day after cessation of berry treatment, both cheek pouches of each group were painted with an 0.2% solution of DMBA in DMSO. Six control animals without berry treatment were painted with 0.2% DMBA+DMSO in the right cheek pouch or DMSO alone in the left pouch. Twenty-four and 48 hrs after DMBA or DMSO treatment, the animals were sacrificed by CO₂ euthanasia and the pouches quick frozen in liquid nitrogen.

DNA was isolated from the left and right cheek pouch tissue of each animal using a direct salt-precipitation method (Miller et al., Nucleic Acid Res. 16:1215 (1988); Schut et al., Cancer Lett. 67:117124 (1992)). ³²P-postlabeling assays for DMBA-DNA adducts were run under intensification conditions (Randerath et al., Carcinogenesis 6: 1117-1126 (1985)). The assay conditions were identical to those used before (50), except for the D3 solvent that was used for the initial separation of adducts (3.5 M lithium formate, 7.0 M urea, pH 3.5) and the D5 solvent (1.0 M magnesium chloride). DNA adduct levels were expressed as relative adduct labeling (RAL) values, after correction of the <RAL> values obtained under intensification conditions.

Induction of Dysplasia in Animal Models: Tissue Collection and Analysis

Carcinogen-induced tissue dysplasia studies were typically carried out as follows. Hamster cheek pouches were painted with 0.2% DMBA in DMSO or DMSO alone 3×/wk for 3 weeks or 3×/wk for 10 weeks with 1% BaP/NNK or DMSO alone. At 3 weeks (DMBA or DMSO) or at 4, 7 and 10 weeks (BaP/NNK or DMSO), cheek pouches from animals that were treated with carcinogen or solvent were harvested and cut longitudinally. One section of each cheek pouch was immediately frozen in liquid nitrogen and stored at −80° C. A second portion of the pouch was fixed in 10% neutral buffered formalin for no more than 8 hrs and paraffin embedded on edge in separate paraffin blocks.

Serial 4 μm sections were cut from formalin-fixed pouches and mounted on Superfrost Plus slides (Fisher Scientific, Pittsburgh, Pa.). A hematoxylin and eosin slide of each HCP was prepared and random tissue sections from each animal were scanned at 100× magnification by an oral pathologist. Each view in field was categorized into one of four histologic categories: normal epithelium, epithelial hyperplasia, low-grade dysplasia, or high-grade dysplasia. The classification scheme utilized was modified from criteria developed by Pozharisski et al. (Tumors of the Esophagus, IARC Scientific Publications, Lyon (1973), pp 87-100) with consideration toward the gross and microscopic descriptions of hyperplasia and dysplasia given in Robbins: Pathologic Basis of Disease. 5th edition.

The samples for the investigations on angiogenesis were obtained from a previous chemopreventive study. Male Fisher rats, 4-5 weeks old, were treated with either NMBA (0.25 mg/kg body weight) or a 1:4 mixture of dimethyl sulfoxide (DMSO):H₂0 (the solvent for NMBA) 3 times per week for 5 weeks. Starting from the 6^{th} week, the NMBA-treated rats were fed with either regular AIN-76A diet or AIN-76A diet containing 5% BRB. At week 25, the animals were sacrificed and esophageal tissues were collected. Half of the esophagus was stored in liquid nitrogen immediately and then transferred to a −80° C. freezer for molecular analysis. The other half esophagus was fixed in 10% neutral buffered formalin for 4 hours, and then transferred to phosphate buffered saline to make paraffin embedded blocks for immunohistochemical analysis.

Real-Time RT-PCR Analysis of iNOS and COX-2

Total cellular RNA was isolated from frozen esophageal tissues using TRIzol Reagent (GIBCO BRL, Gaithersburg, Md.) according to the manufacturer's instructions. After extraction, all RNA samples were analyzed for integrity of 18S and 28S rRNA by ethidium bromide staining of 1 μg of RNA resolved by electrophoresis on 1.2% agarose formaldehyde gels. One-Step Real-Time RT-PCR was performed in a GeneAmp 5700 sequence detection system (Perkin-Elmer Corp., Norwalk, Conn.) using the QuantiTect SYBR Green RT-PCR Kit, from QIAGEN Inc. (Valencia, Calif.) and the experimental protocol provided. A 50 μl reaction volume of total cellular RNA, QuantiTect RT Mix, QuantiTect SYBR Green RT-PCR Master Mix, and forward and reverse primers was reverse transcribed, and then amplified quantitatively by PCR. The iNOS and COX-2 mRNA expression was normalized against mRNA expression of the constitutive gene, hypoxanthine-guanine phosphoribosyltransferase (HPRT). Primers for VEGF, iNOS, COX-2 and HPRT were designed according to published sequences with Primer Express Software V 2.0 (Applied Biosystems, Foster City, Calif.) and were synthesized by Life Technologies, Inc. (Gaithersburg, Md.) or a comparable vendor. Each individual RNA sample for each gene was assayed in triplicate. After the performance of RT-PCR, all data were collected using SDS Sequence Detector Software (PE, Applied Biosystems, Foster City, Calif.).

One experimental protocol that is known to respond properly for this assay used reverse transcription (RT) performed at 50° C. for 30 minutes, followed by polymerase chain reaction (PCR) conditions of 94° C. for 15 seconds, 60° C. for 30 seconds, and 72° C. for 30 seconds for 40 cycles. The expression of VEGF-C and -D mRNA is then normalized against expression of HPRT. The gene expression is expressed as fold change calculated by 2^{-ΔΔC}_{T}. The C_{T} is defined as the PCR cycle number that crossed the threshold. The ΔC_{T} is calculated as “C_{TVEGF}−C_{THPRT}”. The ΔΔC_{T} is calculated as (C_{TVEGF}−C_{THPRT})_{NMBA alone}−(C_{TVEGF}−C_{THPRT})_{NMBA untreated} or (C_{TVEGF}−C_{THPRT})_{NMBA+BRB}−(C_{TVEGF}−C_{THPRT})_{NMBA untreated}.

Assay of iNOS and COX-2 Activity

Frozen rat esophagus samples to be assayed were weighed, homogenized in phosphate buffered saline and centrifuged. iNOS activity in the supernatant was measured using a nitrate/nitrite colorimetric assay kit according to the manufacturer's instructions. For example, 80 μl of supernatant for each sample was pi petted into a 96-well optical plate, and incubated with 10 μl of nitrate reductase and 10 μl of enzyme cofactor for three hours. Griess reagents [sulfanilamide and N-(1-naphthyl)ethylenediamine] were then added and the absorbance was measured at a wavelength of 550 nm using a SpectraMax™ M2 multi-detection reader (Molecular Devise Corp., Sunnyvale, Calif.). Standard solutions of sodium nitrate (0-35 μM) were used to create a standard curve. The final nitrite concentration was the sum of the nitrite plus the reduced nitrate in each sample and was taken as an index of iNOS activity.

COX-2 activity in dissected esophageal epithelium and papillomas was assayed by using Prostaglandin E₂ (PGE₂) Biotrak Enzymeimmunoassay System (Amersham Pharmacia Biotech, Piscataway, N.J.) to measure prostaglandin E₂ concentration. Frozen samples were homogenized in Tris-HCl buffer (pH 7.5) with 0.02 M EDTA and 5 mg/ml indomethacin. Total protein concentration for each tissue homogenate was determined using the DC Protein Assay (Bio-Rad, Hercules, Calif.). Optical density was measured at 450 nm using the SpectraMax™ M2 multi-detection reader. The PGE₂ level was normalized against protein concentration in the same sample.

Cell-Based Assays

Cell-based assays were carried out using art-recognized techniques as described in the examples, and for example, as described in Xue et al., Carcinogenesis, 22:351-356 (2001).

In studies featuring cell lines with reporter genes, typically mouse epidermal cells (i.e., JB-6 clone 41) were stably transfected with either an AP-1-luciferase reporter gene construct (P⁺1-1 cells), a NFκB-luciferase reporter gene construct (CI 41 NFκB mass1 cells), or a p53-luciferase reporter gene construct (CI 41 PG13 mass1 cells) (see, e.g., Huang et al., PNAS 94:11957-11962 (1997); Cancer Res. 57:2873-2878 (1997); and Int J Oncol 13:711-715 (1998)). These resultant cell lines (e.g., CI 41, P⁺1-1, CI 41 NFκB mass1 and CI 41 PG13 mass1) were cultured in Eagle's Minimal Essential Medium (Calbiochem, San Diego, Calif.) supplemented with 5% fetal bovine serum (FBS), 2 mM L-glutamine, and 25 μg of gentamicin/ml (Life Technologies, Inc., Rockville, Md. Cells were cultured at 37° C. in a humidified atmosphere of 5% CO₂ in air. The cultures were dissociated with trypsin and transferred to new 75 cm² culture flasks (Fisher, Pittsburgh, Pa.) from one to three times per week. The substrate for the luciferase assay was obtained from Promega (Madison, Wis.); BPDE was obtained from Sigma (St. Louis, Mo.); and the phospho-specific antibodies against various phosphorylated sites of ERKs, p38 kinase, JNKs, and IκBα were obtained from New England Biolaboratories (Beverly, Mass.). The radiolabel (±)-r-7, t-8-dihydroxy-t-9,10-epoxy-7,8,9,10-tetrahydro[1,3³H]benzo(a)pyrene ([³H]-BPDE, specific activity, 2210 mCi/mmol) was obtained from ChemSyn Science Laboratories (NCI Chemical Carcinogen Repository, Kansas City, Mo.).

AP-1 Activity Assay

The AP-1 activity assay was typically performed using confluent monolayers of P⁺1-1 cells cultured under standard conditions and subsequently incubated with different fractions of black raspberry extract dissolved in DMSO for 30 min at concentrations ranging from 1-100 m/ml. Cells were then exposed to BPDE at a final concentration of 2 μM. The cells were extracted with lysis buffer (Promega, Madison, Wis.) at various periods of time (6-48 h) after BPDE exposure, and the luciferase activity was determined by the Luciferase assay using a luminometer (Wallac 1420 Victor 2 multilable counter system) after the addition of lysis buffer. The results are expressed as AP-1 activity relative to control medium containing DMSO (0.1% v/v) only (Relative AP-1 activity).

NFκB and p53-Dependent Transcription Activity Assays

The same procedure as described above for measuring the effects of berry fractions on BPDE-induced AP-1 activity in P⁺1-1 cells was used for determining the effects of the same berry fractions on BPDE-induced NFκB activity in NFκB mass1 cells, and p53-dependent transcription activity in PG-13 mass1 cells. The results were expressed as either NFκB activity or p53-dependent transcription activity relative to control medium containing DMSO.

### Kinase Phosphorylation Assay

Immunoblots were performed with either phospho-specific antibodies or non-phosphorylated antibodies against various kinases, including ERKs, JNKs and p38 kinase, and also against IκBα. The protein band specifically bound to the primary antibody was detected using an anti-rabbit IgG-AP-linked and an ECF immunoblotting system (Amersham Biosciences, Piscataway, N.J.).

Immunohistochemistry of the Esophagus.

For immunohistochemistry of the esophagus, the whole esophagus was cut into three parts: upper, middle and lower. All three sections were embedded on edge in one block. The paraffin-embedded blocks were serially sectioned at 4 μm and mounted on SuperFrost Plus slides (Fisher Scientific, Pittsburgh, Pa.). As described in Chen, T. and Stoner, G. D. Mol. Carcinog., 40: 232-240 (2004), slides were deparaffinized in histoclear and rehydrated in graded ethanol (100% to 70%). Sections were incubated in order with 3% hydrogen peroxide, casein, goat serum, and avidin and biotin to decrease the nonspecific binding first, and then incubated with mouse monoclonal antibody against CD34 (1:20) for 30 minutes at room temperature. Antibody incubation was followed by 20 minutes incubation with a mouse absorbed link (goat anti-mouse biotinylated immunoglobin) and strepavidin-horseradish peroxidase label. The sections were developed with diaminobenzidine (DAB) chromogen and then counterstained with hematoxylin, dehydrated, and mounted. Reagents were as supplied by BioGenex, Inc., San Ramon, Calif.

Determination of esophageal microvessel density (MVD).

The esophageal MVD was measured by staining sections with antibody specific for CD34 expressed by vascular endothelial cells. Slides were viewed and photographed with a dual-headed Nikon microscope with a high-resolution spot camera, which was interfaced with computer-loaded image analysis software, Simple PCI Imaging Systems (Compix, Inc., Cranberry Township, Pa.). The criteria used to identify microvessels in immunostained sections were established by Folkman et al. (J. Natl. Cancer Inst., 82: 4-6 (1990)). The number of vessels were counted was on an ×200 microscope field. Any brown-staining endothelial cells or endothelial cell clusters that were clearly separate from adjacent blood vessels, tumor cells or other connective tissue elements were considered a single countable microvessel. The distinct clusters of stained endothelial cells that might be from the same vessel snaking its way in and out of the section were considered distinct and countable as a separate microvessel. Vessel lumens were not necessary for a structure to be defined as a microvessel, and red cells were not used to define a vessel lumen. We evaluated the entire esophagus and count microvessel staining positive for CD34 in all areas including normal epithelium, hyperplasia, dysplasia and papillomas allowing the counts to be more representative for each individual esophagus. MVD was calculated using the total number of microvessel in each esophagus dividing by the length of this esophagus, which was expressed microvessels/esophagus length (cm).

Statistical Analysis in Animal Models

Body weight, food consumption, tumor multiplicity (e.g., mean number of tumors/esophagus), and tumor volume data were collected for animals fed with control or experimental diets. Differences between berry fed groups and the control group in the number of tumors were analyzed using Kendall's tau statistics (equivalent to the Mann-Whitney test corrected for ties). In addition, the Fisher exact test was used to examine the dichotomy of having a high versus low number of tumors per animal. The DNA adduct data was evaluated for statistical significance using an ANOVA model accounting for harvest time and LBR. Since a proportionate, rather than absolute, change in response was expected, the response variable in the ANOVA model was on the log scale. In the PBIT and BRB feeding chemoprotective study, differences between groups along with the iNOS and COX-2 mRNA expression data, and the PGE₂ concentration results and 2^{-ΔΔC}_{T}, -ΔC_{T} and MVD values were analyzed for statistical significance using one-way ANOVA followed by Dunnet's multiple comparison test to identify individual differences when the ANOVA was significant. The Spearman's correlation coefficient was used to determine any correlation between the expression of VEGF, COX-2 and iNOS mRNA. Tumor incidence (percent of animals in each group with tumors) data was analyzed using the χ² test. Comparisons of the incidence of esophageal tumors in rats treated with NMBA or a combination of NMBA and PBIT were made using the Kruskal-Wallis test. Software used in this study was GraphPad Prism 4.0. Differences were considered statistically significant at P<0.05. All P values were 2-sided.

### Example 3

### Method of Preparing Berry Extracts

The following studies were performed to determine methods for isolating a fruit or berry extract having desirable properties or producing an extract being enriched for particular components.

Black Raspberry

In preferred embodiments, ripened black raspberries of the Jewel variety were purchased from the Stokes Raspberry Farm (Wilmington, Ohio). The berries were picked mechanically, washed, and placed in a −20° C. freezer within one hour of the time of picking. There are significant changes in the composition of berry extract from fruits that are not preserved by freezing as soon as practicable following harvest from the plants. Frozen berries were then shipped frozen to Van Drunen Farms (Momence, Ill.) for freeze-drying, and subsequently were ground into a powder as while kept cooled. The berry powder was shipped frozen to The Ohio State University where it was kept at −20° C. until used in experiments.

The powder prepared according to the method was analyzed for composition, including for concentration of several vitamins, minerals, carotenoids and simple polyphenols by Covance Laboratories and for anthocyanin composition as described in Tian, et al., Food Chemistry 94 465-468 (2006). Table 3 shows analysis of Black Raspberry extract from three separate lots, and demonstrates expected compositions of fruit handles according to the standard process ing steps employed with the invention. In general, so long as harvested berries are processed as described, the composition does not vary substantially from those compositional ranges shown in Table 3. For instance, the contents of berries harvested and processed in the year 2002 were found to vary no more than 20% from those of black raspberries obtained from the same source in previous years. Failure to adhere to the described protocol, wherein the fruits are frozen as soon as practicable, can result in substantial degradation of the beneficial phtyochemicals described herein, even though the overall quality of the fruits appears suitable for human consumption.

TABLE 3
Composition of Cultivars of Black Raspberry
Extracts used in Chemoprevention Studies

| | Identification of berry lot | | |
|---|---|---|---|
| Components^{a} | LBR98^{b} | LBR95^{c} | LBR97^{d} |

Minerals

| | | | |
|---|---|---|---|
| Calcium | 170.00 | 245.00 | 215.00 |
| Copper | 0.74 | 0.52 | 0.55 |
| Iron | 4.95 | 13.20 | 10.10 |
| Magnesium | 147.00 | 169.00 | 153.00 |
| Manganese | 5.85 | 3.60 | 4.68 |
| Phosphorus | 168.00 | 222.00 | 170.00 |
| Potassium | 1060.00 | 1200.00 | 1300.00 |
| Sodium | <10.00 | <10.00 | <10.00 |
| Zinc | 2.12 | 2.69 | 2.12 |
| Selenium | <5.00 | <5.00 | <5.00 |

Vitamins

| | | | |
|---|---|---|---|
| Folic Acid | 0.51 | 0.07 | 0.06 |
| Vitamin C | <1.00 | <1.0 | 4.14 |

Sterols

| | | | |
|---|---|---|---|
| β-sitosterol | 72.40 | 89.10 | 80.10 |
| Campesterol | 4.60 | 4.30 | 3.40 |
| Cholesterol | <1.00 | <3.00 | <1.00 |
| Stigmasterol | <3.00 | <1.00 | <3.00 |

Phenolics

| | | | |
|---|---|---|---|
| Ellagic Acid | 200.00 | 185.00 | 166.30 |
| Ferulic Acid | 21.00 | 32.40 | 17.60 |
| Anthocyanins | 1770 | NT | NT |
| p-Coumaric Acid | 6.72 | 7.94 | 9.23 |

Carotenoids

| | | | |
|---|---|---|---|
| α-carotene | <0.02 | <0.02 | <0.02 |
| β-carotene | 0.12 | <0.02 | <0.02 |
| Lutein | <0.02 | <0.02 | <0.02 |
| Zeaxanthin | <0.02 | <0.02 | <0.02 |

^{a}Concentration of components is expressed as mg/100 g of LBR: selenium is expressed as μg/100 g.
^{b}Lot used for inhibition of oral tumors in HCP.
^{c}Lot used for inhibition of esophageal tumors in rats (complete carcinogenesis bioassay, Kresty et al., Cancer Res. 61: 6112-6119 (2001)
^{d}Lot used for inhibition of esophageal tumors (post -initiation assay, ref. #46)

Freeze-dried (lyophilized) black raspberries (Rubus occidentalis) and strawberries (Fragaria ananassa) puree of pulp with seeds were also prepared as follows. Several hundred pounds of fresh, ripe black raspberries and strawberries were picked, washed, and stored frozen at −20° C. Berry puree, free of cap stems and seeds, was prepared by passing the whole berries through a pulper-finisher fitted with a screen having 0.020-inch perforations. The waste fraction was returned to the pulper three times to assure complete juicing of the harder white shoulders of the berries. The seed was pulverized and added to the puree. The puree containing pulverized seed was poured to a depth of approximately 1 inch into freeze-dryer trays lined with polyethylene film, and then frozen in a blast freezer. The frozen plates of puree were removed and stored at −20° C. for subsequent freeze drying.

Freeze-drying was accomplished by means of a Virtis model 50-SRC-5 Sublimator. The shelf temperature was 40° C. and the vacuum was 380 millitorr. One defrost cycle was required for each batch containing about 70 pounds of puree. Approximately three days are required to dry each batch of puree. When dry, the thickest portion of each plate of dried material was visually checked for remaining ice. If ice was found, freeze-drying was continued. When the product was found to be dry, it was packaged in doubled polyethylene bags, placed in carton boxes, and stored at −20° C.

The berry extracts were then used as the source material for further analysis and fractionation described herein.

### Example 4

### Analysis of the Components of Berry Extracts

In this example, the berry extracts, isolated using the methods of the invention described above, were subjected to a detailed analysis of its beneficial components.

In particular, samples of freeze-dried strawberries and black raspberries prepared as described above where analyzed for their overall antioxidant activity as well as the presence of selected vitamins, carotenoids, phenolic compounds, phytosterols and minerals. First, the overall antioxidant activity for each extract was determined using techniques described herein and results are shown in Table 4.

TABLE 4
Oxygen Radical Absorbance Capacity (ORAC)

| Extract | ORAC Value (per mg) |
|---|---|
| Strawberry | 15.36* |
| Black Raspberry | 16.09 |

*= estimate based on Wang et al. Agr. Found. 44: 701-705 (1996).

The content of selected vitamins, carotenoids, phenolic compounds, phytosterols, and minerals, was then determined for several extract samples of each harvest lot. Strawberry extracts (prepared either from fresh strawberries or strawberries that were frozen at −20° C. for either 24 hours or several months following harvest) were analyzed and results are shown in Table 5. All strawberry extract components in Table 5 were well preserved for a period of at least one year after the berries were freeze-dried and maintained at either −20° C. or at refrigerator temperature (4° C.). One exception is Vitamin C composition. The Vitamin C composition in fresh strawberries is well preserved in freeze-dried material if the berries are freeze-dried within 24 hours following harvest from the field. In contrast, when the strawberry fruits are stored frozen in an intact state at −20° C. for several months following harvest, the Vitamin C content was markedly reduced compared to fruits that were harvested, frozen and then freeze dried after 24 hours. Thus, the degradation of Vitamin C in strawberries when the berries are stored for several months at −20° C. before freeze-drying indicates a loss of quality during storage of fresh-frozen fruits. As a number of the constituents of fruit are labile, and may degrade similarly to that of Vitamin C, it is preferable that the fruit be field chilled before significant degradation of active constituents may occur. Chilling within four hours is preferred, and chilling within two hours of picking is even more preferred. By the time a reduction in quality of gross fruit characteristics is noticeable, significant degradation of individual active constituents is likely to have occurred.

TABLE 5
Components of Strawberry Extracts

| | | | |
|---|---|---|---|
| | | Freeze-Dried | Freeze-Dried |
| | Fresh | Fresh | Fresh-Frozen |
| | Strawberries | Strawberries | Strawberries |
| Component | (mg/kg)^{a} | (mg/100 g)^{a} | (mg/100 g)^{b} |

Vitamins

| | | | |
|---|---|---|---|
| Vitamin A | 425.00 | 267.00 | —^{c} |
| Vitamin E | 5.86 | 4.95 | —^{c} |
| Vitamin C | 348.50 | 371.00 | 141.00 |
| Folic Acid | 0.60 | 0.59 | 0.37 |

Carotenoids

| | | | |
|---|---|---|---|
| α-carotene | <0.02 | <0.02 | 0.03 |
| β-carotene | 0.25 | 0.16 | <0.02 |
| Zeaxanthin | <0.02 | <0.02 | <0.02 |
| Lutein | 0.17 | 0.11 | <0.02 |

Phenolic compounds

| | | | |
|---|---|---|---|
| Ellagic acid | —^{c} | 67.00 | 140.00 |
| Ferulic acid | <2.50 | <2.5 | <2.5 |

Phytosterols

| | | | |
|---|---|---|---|
| β-sitosterol | 39.10 | 40.70 | 27.20 |
| Campesterol | <3.00 | <3.00 | <3.00 |
| Stigmasterol | <3.00 | <3.00 | <3.00 |

Minerals

| | | | |
|---|---|---|---|
| Calcium | 92.65 | 72.40 | 160.00 |
| Magnesium | 109.65 | 91.90 | 124.00 |
| Potassium | 1445.00 | 1110.00 | 1640.00 |
| Zinc | 0.93 | 0.63 | 0.99 |
| Selenium | <0.01 | <0.01 | 11.00 |

Fiber

| | | | |
|---|---|---|---|
| Fiber | ~5% of total wt. | ~45% of total wt. | ~45% of total wt. |

^{a}11/00 harvest. Fresh strawberries were frozen at −20° C. immediately after purchase from a store. Some were kept frozen and the remaining berries were freeze-dried. Both the fresh berries and freeze-dried berries were analyzed for various components
^{b}10/95 harvest. Stored frozen several months before freeze drying. Then stored in a refrigerator for one year before analysis.
^{c}not analyzed
^{d}approximate value

In Table 6, the content of selected vitamins, carotenoids, phenolic compounds, phytosterols, and minerals, in black raspberry extracts is shown. Some of these data were presented in Table 3. In both samples, the black raspberries were stored frozen at −20° C. for at least six months before they were freeze-dried. As indicated, the vitamin C content of the black raspberries is low suggesting that it degraded during storage at −20° C. The contents of the other berry components is well preserved for more than one year when stored at refrigerator temperature (4° C.).

In particular, the oxygen radical absorbance capacity (ORAC) assay was demonstrates the presence of antioxidant activity. Both freeze-dried black raspberry and strawberry extracts were tested for antioxidant activity using the ORAC assay and the ORAC values for both fruit types was elevated. Thus, an enrichment of 5 fold or even 10 fold of the ORAC value can serve as an indicator that the therapeutic value, including anti-cancer activity, of a fruit extract has increased over that available in the naturally occurring fruit. While a five fold increase in ORAC value per milligram of material provided would not necessarily be directly correlated with a 5 fold increase in anti cancer activity, it is known that in simple extractions, the antioxidant activity, as demonstrated by ORAC values, is correlated with therapeutic value of the extract.

In another approach for determining the antioxidant activity of the berry extracts of the invention, electron spin resonance technology was used. Each fraction was evaluated for its ability to quench singlet oxygen and hydroxide ion (electron spin resonance (ESR)). The fractions exhibit varying abilities to quench these free radicals, and overall, are highly active when compared to control compounds with high levels of antioxidant activity.

TABLE 6
Components of Black Raspberry Extracts

| | | |
|---|---|---|
| | Black Raspberries | Black Raspberries |
| Substance | (mg/100 g)^{a} | (mg/100 g)^{b} |

Vitamins

| | | |
|---|---|---|
| Vitamin A | —^{c} | —^{c} |
| Vitamin E | —^{c} | 10.80 |
| Vitamin C | < 1.00 | <0.10 |
| Folic Acid | 0.07 | 0.013 |

Carotenoids

| | | |
|---|---|---|
| α-carotene | <0.02 | <0.02 |
| β-carotene | <0.02 | 0.012 |
| Zeaxanthin | <0.02 | <0.04 |
| Lutein | <0.02 | 0.03 |

Phenolic Compounds

| | | |
|---|---|---|
| Ellagic acid | 175.00 | 200.00 |
| Ferulic acid | 32.40 | 21.00 |
| Anthocyanins | —^{c} | 1770.00 |

Phytosterols

| | | |
|---|---|---|
| β-sitosterol | 89.10 | 72.40 |
| Campesterol | 4.30 | 4.60 |
| Stigmasterol | <3.00 | <3.00 |

Minerals

| | | |
|---|---|---|
| Calcium | 245.00 | 167.00 |
| Magnesium | 169.00 | 147.00 |
| Potassium | 1200.00 | 1060.00 |
| Zinc | 2.70 | 2.12 |
| Selenium | <5.00 | <0.01 |

Fiber

| | | |
|---|---|---|
| Fiber | ~45% of total | ~45% of total |
| | freeze dried wt. | freeze dried wt. |

^{a}12/95 harvest. Stored frozen several months before freeze-drying. Then
stored in refrigerator for one year before analysis.
^{b}7/98 harvest. Stored frozen several months before freeze-drying. Then
stored in refrigerator for one year before analysis.
^{c}not analyzed

For each fruit extract tested, beneficial compounds such as vitamins (e.g., Vitamin E, Vitamin C, and folic acid); carotenoids (e.g., α-carotene, β-carotene, zeaxanthin, lutein); phenolic compounds (e.g., ellagic acid, ferulic acid, and anthocyanins); phytosterols (e.g., β-sitosterol, campesterol, and stigmasterol, and analogs thereof); and minerals (e.g., calcium, magnesium, potassium, zinc, and selenium) were detected. The raspberry extracts of the invention are particularly enriched for the presence of antioxidant activity.

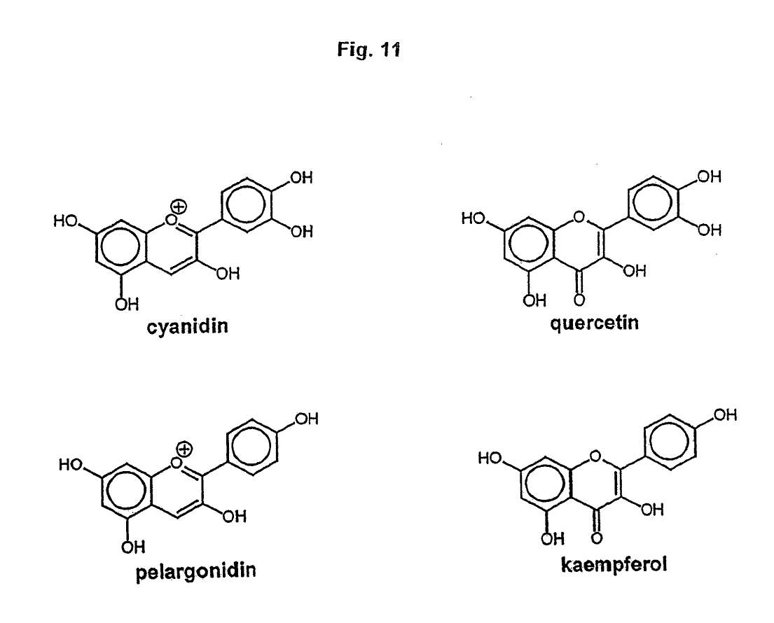

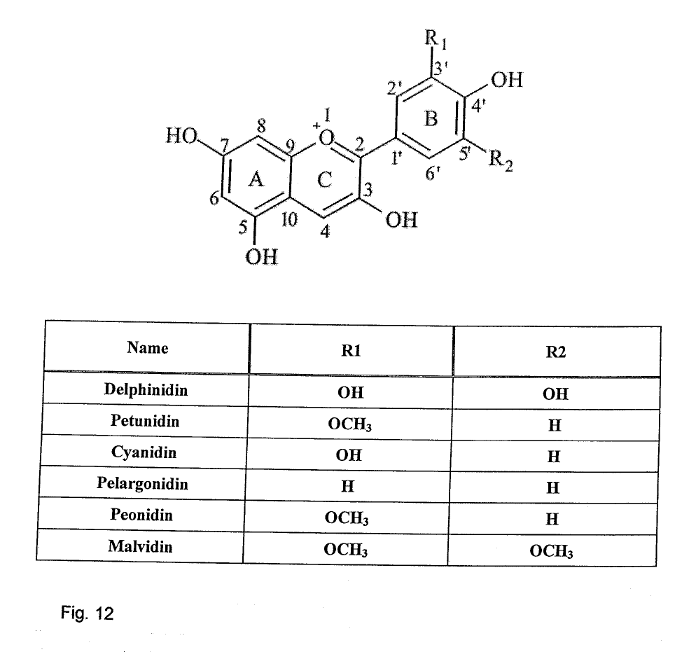

In addition, upon further fractionation of the black raspberry extracts in particular, several bioactive components were identified. Specifically, the methanol extract of freeze-dried black raspberries was further studied, as this fraction had the most activity in inhibition of cellular transformation and down regulation of AP-1 and NFκB activities, as discussed herein. Analysis of this fraction by HPLC with UV detection, using a C18 reverse-phase system, gave the chromatogram illustrated in FIG. 11. Using diode-array detection, UV spectra were obtained on all peaks. Liquid chromatographyelectrospray ionization-negative ion-mass spectrometry (LC-ESI-MS) analysis of this fraction, with selected ion monitoring for the glycosides of 4 flavonoids known to be present in raspberries: cyanidin, quercetin, pelargonidin, and kaempferol, was carried out. The structures of these compounds are illustrated in FIG. 12.

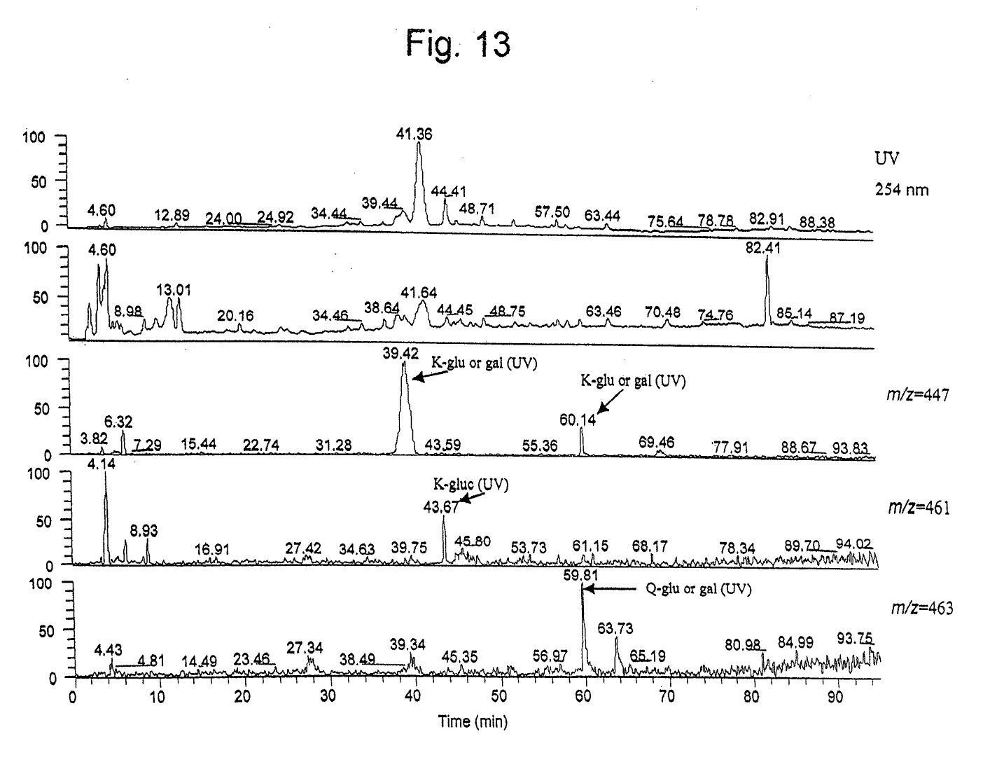

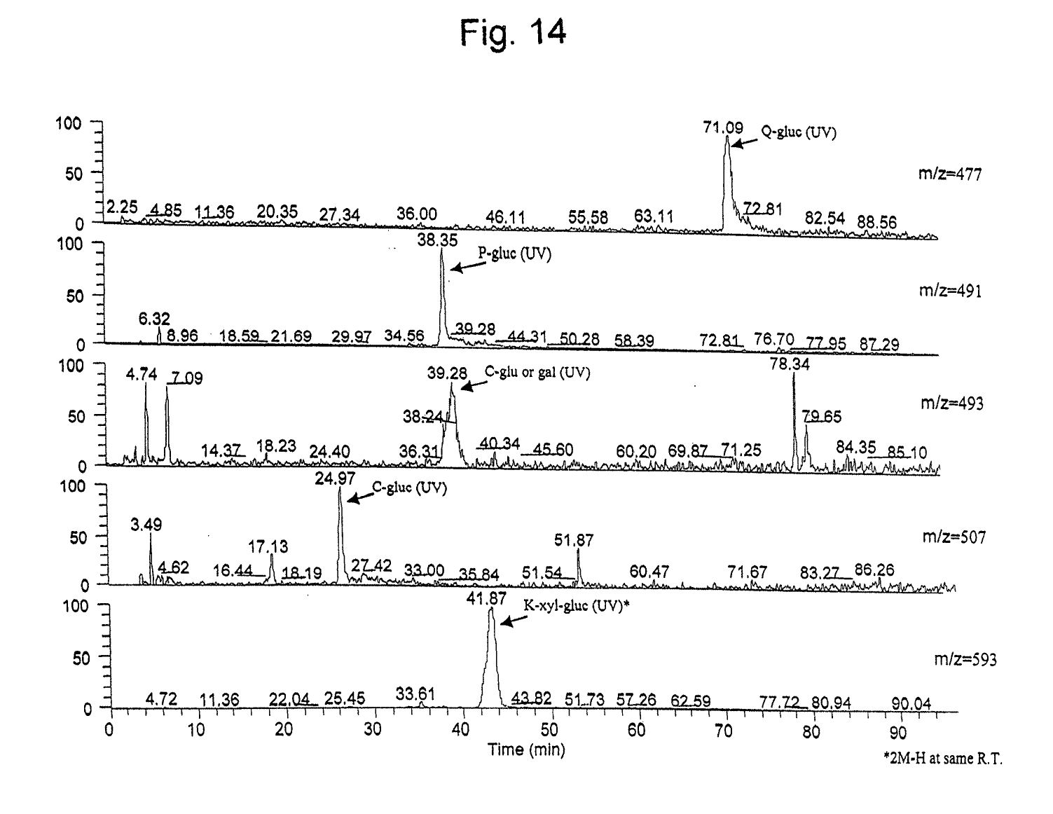

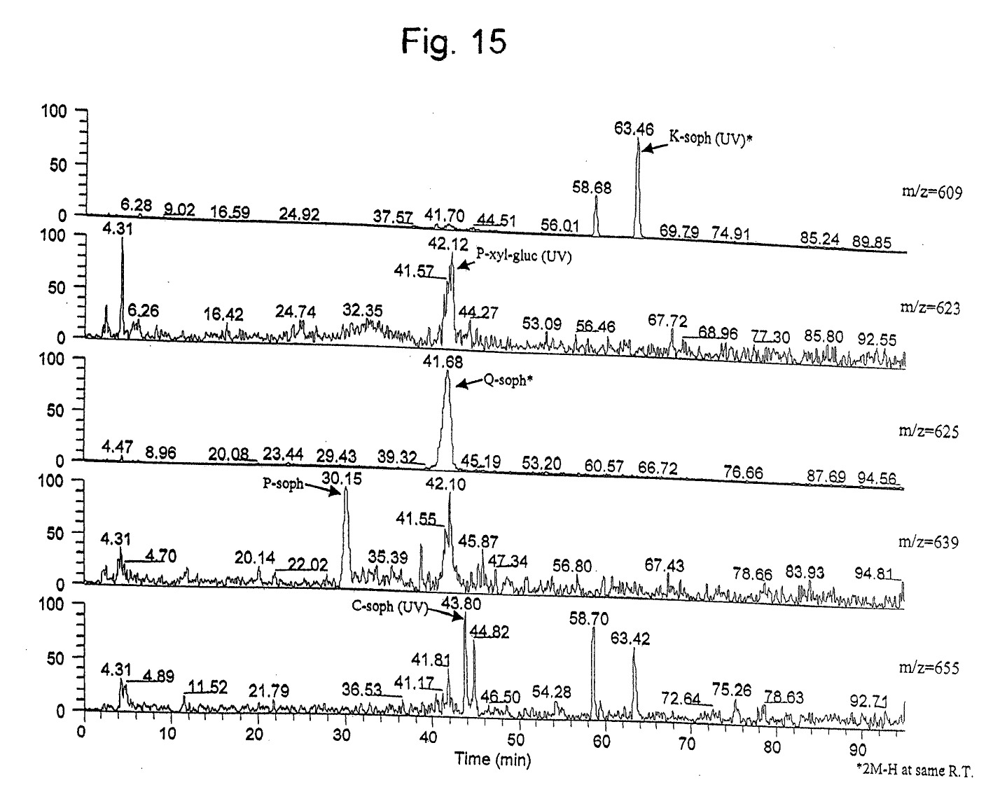

Analyses of standards demonstrated that M−1 peaks and 2M−1 peaks would be obtained under these conditions. In addition, the following known sugar conjugates of cyanidin, quercetin, pelargonidin, and kaempferol: glucoside, galactoside, glucuronide, sophoroside, and xylosylglucuronide were selected for ion monitoring analysis as shown in FIGS. 13-15. The top panel of FIG. 13 shows the UV trace, and the second panel shows the chromatogram obtained when monitoring total ion current. The third panel shows the chromatogram obtained by monitoring m/z 447, M-1 of kaempferol glucoside and galactoside. The peaks marked K-glu or gal (UV) also had UV spectra and MS consistent with kaempferol glucoside. Similarly, the fourth panel shows the results of selected ion monitoring for m/z 461, which is M-1 of kaempferol glucuronide. The peak marked K-gluc (UV) had UV and MS consistent with kaempferol-glucuronide. The fifth panel shows selected ion monitoring for m/z 463, M-1 of quercetin glucoside or galactoside. The peak marked Q glu or gal (UV) had UV and MS consistent with queretin glucoside or galactoside. FIGS. 14 and 15 show similar data, where P refers to pelargonidin, C refers to cyanidin and Q refers to quercitin.

Collectively, these data demonstrate the presence of desirable flavonoids in the active fraction of freeze-dried black raspberries.

### Example 5

### Cell Based Study Demonstrating Anti-Cancer Properties of Alcohol Fruit Extract Fractions

The following studies were performed to examine the anti-cancer properties of the berry extracts of the invention and fractions derived therefrom.

Briefly, black raspberry extract fractions (RU-F001, RU-F003, RU-F004, RU-F005, RU-DM, RU-ME) and strawberry extract fractions (FA-F001, FA-F003, FA-F004, FA-F005, FA-DM, FA-ME) isolated as described above were analyzed for anti-transformation activity in the Syrian hamster embryo (SHE) cell transformation model using benzo(a)pyrene (B[a]P) as the chemical carcinogen. None of the extract fractions by themselves produced an increase in morphological transformation. For assessment of chemopreventive activity, SHE cells were treated with each extract fraction at doses ranging from 2-100 microgram per milliliter and B[a]P (10 microgram per milliliter) for seven days. The RU-ME and FA-ME extract fractions isolated as described above produced a dose-dependent decrease in transformation as compared to B[a]P treatment only.

The raspberry extract fraction (RU-ME) and strawberry extract fraction (FA-ME) were further examined using a 24 hour co-treatment with B[a]P or a 6 day treatment following a 24 hour treatment with B[a]P. Both extract fractions significantly reduced B[a]P-induced transformation when co-treated with B[a]P for 24 hours. These results indicate that the methanol fractions from black raspberry extracts and strawberry extracts inhibit cell transformation through interference of the uptake, activation and/or detoxification of B[a]P and/or intervention of DNA binding and DNA repair.

### Example 6

### Fruit Extract Fractions Inhibit the Formation of DNA Adducts

The following studies were performed to examine the ability of the fruit extracts fractions to inhibit the formation of DNA adducts in vivo. In this example, the hamster cheek pouch (HCP) animal model as described above was used to evaluate the ability of black raspberries to inhibit the formation of DNA adducts in the check pouches of animals treated with the cancer inducing agent, DMBA. Under intensification conditions and using the ³²P-postlabeling technique, a total of four DNA adducts could be detected in the cheek pouches of animals treated with DMBA. After running the assay under standard (ATP-saturating) conditions, intensification factors for adducts 1, 3, and 4 were found to be 37.7, 8.1, and 10.5, respectively. A minor adduct (#2) was not detectable under standard assay conditions as it amounted to only 1.2-4.3% of the total intensified adducts (<RAL> values), except for four separate samples where it constituted 7.1-9.8% of the total. Of the total corrected adducts (RAL values), adducts 1, 3, and 4 constituted 38.8-59.0%, 21.3-35.9%, and 17.8-29.0%, respectively, of the adduct burden. For quantitative comparisons, total RAL's (sum of adducts 1, 3, and 4) and sum of specific adducts were used. The 5% berry diet inhibited DMBA adducts by 29% and 55% (mean total adduct levels) at 24 and 48 hr (Table 7 below) with a statistical significance of p=0.07. Similar differences between berry and DMBA control groups for the formation of other adducts was observed.

TABLE 7
Inhibition of DMBA Adducts by Lyophilized Black Raspberries

| | | DNA adducts (RAL × 10⁷)^{c} | | | |
|---|---|---|---|---|---|
| | | Adduct | Adduct | Adduct | |
| Treatment^{a} | Harvest^{b} | 1 | 3 | 4 | Total SEM^{d} |
| 5% LBR + | 24 hr | 17.95 | 12.24 | 9.52 | 39.7 +/− 10.7 |
| DMBA | | | | | |
| Control + | | 26.50 | 15.88 | 13.09 | 55.5 +/− 16.2 |
| DMBA | | | | | |
| Control + | | 0.9441 | 0.4079 | 0.3467 | 1.70 +/− 0.34 |
| DMSO | | | | | |
| 5% LBR + | 48 hr | 10.84 | 6.87 | 5.52 | 23.2 +/− 11.8 |
| DMBA | | | | | |
| Control + | | 24.02 | 14.11 | 13.10 | 51.2 +/− 3.8 |
| DMBA | | | | | |
| Control + | | 0.5226 | 0.2129 | 0.2102 | 0.95 +/− 0.11 |
| DMSO | | | | | |

^{a}Hamsters were given 5% LBR or AIN-76A control diet for 48 hr prior to DMBA challenge: DMBA was given as a single dose by painting the HCP with 0.2% DMBA in DMSO.
^{b}Twenty-four and 48 hr after DMBA treatment, cheek pouches were harvested from euthanized animals and immediately frozen in LN₂.
^{c}Relative Adduct Labeling under intensification conditions (ref. 51). A minor DMBA adduct (#2) is not shown in the table, as it represented only 1.2-4.3% of the total intensified adducts.
^{d}Total adduct burden. Sum of RAL of adducts 1, 3, and 4 ± standard error of mean.
^{e} Overall difference between 5% berry treated and DMBA control animals was only marginally significant.

This study indicates one mechanism by which the berry extracts of the invention reduce cancer, i.e., tumor burden, is that the extracts (LBR) inhibit the formation of pro-mutagenic adducts formed by DMBA. In short term bioassays, feeding of both 5% and 10% berries prior to a single carcinogen treatment with 0.25 mg/Kg NMBA resulted in 73% and 80% reductions in O⁶-methylguanine adducts in esophageal tumorgenesis (Kresty et al., Cancer Res. 61: 6112-6119, 2001.). In the HCP, when hamsters were given 5% LBR for two weeks prior to DMBA challenge, three major adducts (adducts 1, 3, 4) were found to be inhibited by 29% when analyzed 24 hr after DMBA treatment. When analyzed 48 hr after DMBA treatment, the inhibition of DMBA-DNA adducts by 5% berries was greater than 50%. Therefore, the observed decrease in HCP tumors can be explained, in part, by the inhibition of DNA adduct formation.

In summary, the chemoprevention studies presented herein show that incorporation of black raspberries in the diet will inhibit tumor formation in the oral mucosa of mammals. As the present disclosure demonstrates, the therapeutic benefit is enhanced by incorporating a purified version of the fruit extract into compositions that will limit the inclusion of unnecessary calories present in the fruit sugar, and obviate the need to consume large quantities of fruit of lyophilized fruit.

### Example 7

### In Vivo Method for Determining the Anti-Cancer Properties of Fruit Extracts on Tobacco Related Oral Cancer

The following example demonstrates the ability of the fruit extracts to inhibit the formation of cancer caused by tobacco use. In order to develop an oral cancer model that would mimic the conditions found in human oral mucosa after being exposed to exogenous tobacco carcinogens (e.g., polycyclic aromatic hydrocarbons (PAHs) and nitrosoamines), the cheek pouch of a model animal (i.e., hamsters, hamster cheek pouch (HCP); 2-3 animals per group) was painted with a reduced total dose of DMBA (0.2% DMBA in DMSO, 3×/wk for three weeks) or with a 1% BaP/NNK mixture (3×/wk for 10 wk). Twenty-four hours after the final DMBA treatment or at 4, 7, and 10 wk of BaP/NNK treatment, hamsters were sacrificed and the HCPs were divided longitudinally into two sections, one for quick freezing and the second for histological examination. Control tissues, that were treated only with DMSO solvent, had a normal histologic appearance with a normal orthokeratin pattern and no evidence of a hyperproliferative or inflammatory response upon histological examination. Histopathologic examination of sections taken 24 hrs after the last DMBA treatment showed morphologic changes ranging from a mild inflammatory response to areas of focal dysplasia, as evidenced by abnormal cell maturation, increased mitotic figures, and cellular pleomorphism.

Control hamster cheek pouch epithelium treated for 10 weeks with the DMSO vehicle, showed a uniform histology characterized by a 3-4 epithelial cell thickness, lack of defined epithelial rete ridges, hyperorthokeratosis, and un-inflamed connective tissue upon histological examination. In contrast, within 7 weeks after three times/week of 1% NNK/BaP topical application, the surface epithelium showed a slight basilar hyperplasia, increased thickness of the spinous layer (acanthoid), and a mild chronic inflammatory cell infiltrate in the superficial connective tissue upon histological examination. Ten weeks after NNK/BaP application, the experimental animals showed histologic evidence of epithelial dysplasia similar to the dysplastic epithelial progression in human oral mucosa, i.e., maturational perturbations began in the basilar third of the hamster epithelium. These tissues, upon histological examination, also evidenced tear-dropped shaped epithelial rete ridges in conjunction with basilar hyperplasia, consistent with moderate epithelial dysplasia.

Accordingly, the above animal model is suitable for determining the cancer inhibiting properties of the extracts described herein. In particular, for evaluating the ability of black raspberry extracts to inhibit oral cavity tumors caused by long term tobacco use. Male Syrian Golden hamsters, 3-4 weeks of age, can be fed 5% and 10% lyophilized black raspberries (LBR) in the diet for two weeks prior to treatment (and/or during or after treatment) with a cancer inducing agent as described above. Diets comprising 5% and 10% lyophilized black raspberries (LBR) prepared as described above and determined to comprise the following components as indicated above (Tables 3, 5) can be used. The cancer agent can be applied to the oral cavities of the animals for eight weeks after which the animals were sacrificed 12-13 weeks from the beginning of treatment and the number and volume of tumors (mm³) can be determined and/or histological examination is conducted on tissue samples of the oral cavity. Significant differences in the number, volume, or incidence of tumors or the degree of tissue dysplasia determined by histological examination are evaluated in animals fed a fruit extract and or a fruit extract fraction as compared to control animals

Accordingly, the chemoprevention studies above, using a mixture of the tobacco-associated carcinogens, BaP and NNK, provide the ability to evaluate the anti-cancer properties of the berry extracts of the invention in a well-defined animal system that mimics the pathologic condition of former tobacco users.

The following studies are performed to examine the ability of the fruit extract fractions to inhibit the formation of cancer caused by tobacco use. In order to develop an oral cancer model that would mimic the conditions found in human oral mucosa after being exposed to exogenous tobacco carcinogens (e.g., polycyclic aromatic hydrocarbons (PAHs) and nitrosoamines), the cheek pouch of a model animal (i.e., hamsters, hamster cheek pouch (HCP); 2-3 animals per group) was painted with a reduced total dose of DMBA (0.2% DMBA in DMSO, 3×/wk for three weeks) or with a 1% BaP/NNK mixture (3×/wk for 10 wk). Twenty-four hours after the final DMBA treatment or at 4, 7, and 10 wk of BaP/NNK treatment, hamsters were sacrificed and the HCPs were divided longitudinally into two sections, one for quick freezing and the second for histological examination. Control tissues, that were treated only with DMSO solvent, had a normal histologic appearance with a normal orthokeratin pattern and no evidence of a hyperproliferative or inflammatory response upon histological examination. Histopathologic examination of sections taken 24 hrs after the last DMBA treatment showed morphologic changes ranging from a mild inflammatory response to areas of focal dysplasia, as evidenced by abnormal cell maturation, increased mitotic figures, and cellular pleomorphism.

Control hamster cheek pouch epithelium treated for 10 weeks with the DMSO vehicle, showed a uniform histology characterized by a 3-4 epithelial cell thickness, lack of defined epithelial rete ridges, hyperorthokeratosis, and un-inflamed connective tissue upon histological examination. In contrast, within 7 weeks after three times/week of 1% NNK/BaP topical application, the surface epithelium showed a slight basilar hyperplasia, increased thickness of the spinous layer (acanthoid), and a mild chronic inflammatory cell infiltrate in the superficial connective tissue upon histological examination. Ten weeks after NNK/BaP application, the experimental animals showed histologic evidence of epithelial dysplasia similar to the dysplastic epithelial progression in human oral mucosa, i.e., maturational perturbations began in the basilar third of the hamster epithelium. These tissues, upon histological examination, also evidenced tear-dropped shaped epithelial rete ridges in conjunction with basilar hyperplasia, consistent with moderate epithelial dysplasia.

Accordingly, the above animal model is suitable for determining the cancer inhibiting properties of the extracts described herein. In particular, for evaluating the ability of black raspberries to inhibit oral cavity tumors caused by long term tobacco use. Male Syrian Golden hamsters, 3-4 weeks of age, can be fed 5% and 10% lyophilized black raspberries (LBR) in the diet for two weeks prior to treatment (and/or during or after treatment) with a cancer inducing agent as described above.

Diets comprising 5% and 10% lyophilized black raspberries (LBR) prepared as described above and determine to comprise the following components as indicated above (Tables 3, 5) can be used.

The cancer agent can be applied to the oral cavities of the animals for eight weeks after which the animals were sacrificed 12-13 weeks from the beginning of treatment and the number and volume of tumors (mm³) can be determined and/or histological examination is conducted on tissue samples of the oral cavity. Significant differences in the number, volume, or incidence of tumors or the degree of tissue dysplasia determined by histological examination are evaluated in animals fed a berry extract as compared to control animals

Accordingly, the chemoprevention studies above, using a mixture of the tobacco-associated carcinogens, BaP and NNK, provide the ability to evaluate the anti-cancer properties of the berry extracts of the invention in a well-defined animal system that mimics the pathologic condition of former tobacco users.

### Example 8

### Cell Based Method Demonstrating the Anti-Colon Cancer Activity of Fruit Extract Fractions

The following disclosure demonstrates the anti-cancer properties of the fruit extract fractions in human colon carcinoma. In this example, the growth inhibitory effects of anthocyanin-rich black raspberry extracts on the growth of normal and cancerous human colon cell lines were examined. In particular, anthocyanin-rich extracts from black raspberries (Rubus occidentalis) were investigated for their inhibitory properties on the proliferation of normal colon cell lines and cancerous colon cell lines (HT-29). As shown in Table 8, All extracts (i.e., fractions DM, F001, F003, F004, and ET) inhibited the proliferation of the human colon cancer cell line, HT-29, within 24 h of administration of the extract. Notably, colon cancer cells were more susceptible to growth inhibition by anthocyanin-rich extracts at concentrations of 5 to 50 μg/ml than normal human colon cells. Cell cycle analyses indicated that progression through the cell cycle was altered in extract-treated cells as compared to untreated controls.

These findings indicate that the anthocyanin-rich berry extracts of the invention can inhibit the growth of human colon cancer cells.

TABLE 8
Growth Inhibition of Human Colon Carcinoma
Cells by Extract Fractions

| % Inhibition | | | Fraction-in |
|---|---|---|---|
| 24 h | 72 h | 6 d | μg/ml |
| 0 | 0 | 0 | DM-0 |
| 42.23 | 9.031 | 6.843 | DM-05 |
| 42.72 | 24.215 | 2.87 | DM-25 |
| 45.63 | 33.639 | 7.947 | DM-50 |
| 0 | 0 | 0 | F001- 0 |
| 3.07 | −0.386 | 33.483 | F001- 05 |
| 16.667 | 42.6 | 30.562 | F001-25 |
| 40.789 | 37.58 | 35.506 | F001-50 |
| 0 | 0 | 0 | F003- 0 |
| 13.0653 | 47.607 | 8.913 | F003- 05 |
| 4.5226 | 50.453 | 18.004 | F003-25 |
| 3.518 | 48.124 | 24.777 | F003-50 |
| 0 | 0 | 0 | F004-0 |
| 37.433 | 12.87 | 29.448 | F004-05 |
| 47.594 | 16.86 | 40.286 | F004-25 |
| 44.92 | 19.949 | 37.014 | F004-50 |
| 0 | 0 | 0 | ET-0 |
| 32.738 | −6.7 | 29.4334 | ET-05 |
| 33.929 | 5.583 | 30.189 | ET-25 |
| 35.119 | 16.005 | 38.868 | ET-50 |

### Example 9

### Cell Based Method Demonstrating the Anti-Oral Cancer Activity of Fruit Extract Fractions

The following disclosure demonstrates the anti-oral cancer properties of the fruit extract fractions of the invention in human oral carcinoma. In particular, using a panel of normal, premalignant and malignant oral epithelial cell lines, the cellular (growth inhibiting and cytotoxic) effects of phytochemicals found in black raspberry fractions was determined (i.e., F001, F003, DM, and ME/Et, see FIGS. 2A and 2B). The fruit extract representing ˜55% of the total berry components (F001) did not affect the growth or induce cytotoxicity in the oral cell lines. However, partitioning and chromatography of the F001 extract yielded three fractions which exhibited varying degrees of growth inhibition in the oral cell lines. The water soluble F003 fraction exhibited no growth inhibiting effects to any the cell lines. The F001 berry fraction pardoned into chloroform (F003) was selectively growth inhibitory to the premalignant oral cell line. Following silica gel column chromatography of the F001 extract, the fractions eluting with dichloromethane (DM) and methanol/ethanol (Me/Et) were selectively growth inhibitory to the premalignant and malignant cell lines. The extracts or fractions were not observed to be cytotoxic to the cells, indicating that phytochemicals in the DM, ethanol and methanol extracts are growth inhibitory without eliciting cytotoxicity. Coinciding with the selective growth inhibiting effects of DM and Et, the number of premalignant and malignant cells increased in the G₂/M phase of the cell cycle. Ellagic acid, a major component of berries, was tested and found to be a potent, non-selective, inhibitor of oral human cell growth. These studies demonstrate that non-toxic doses of berry extracts, and components thereof, such as ellagic acid, are capable of inhibiting the proliferation of human oral precancerous and cancer cells.

### Example 10

### Effects of Black Raspberries Supplements on Angiogenesis

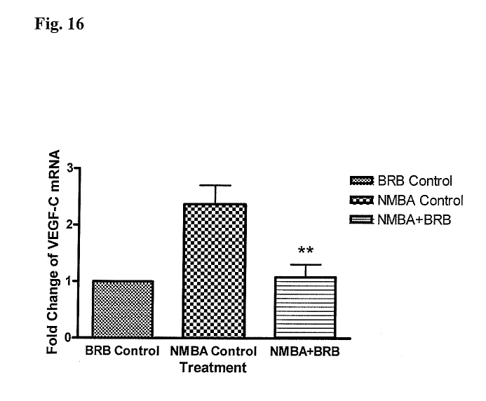

Supplementation of the animal diet with BRB composition down-regulates VEGF mRNA Expression and alters the density of microvessels and microvessel development. As shown in FIG. 16 both VEGF-C and VEGF-D mRNA were assessed by Real-Time RT-PCR to demonstrate the anti-angiogenic effects of dietary BRB supplements. The VEGF-D mRNA expression of rat esophagus were not substantially altered after rats were treated with NMBA. In contrast, VEGF-C mRNA expression was significantly elevated in rats treated with NMBA when compared to untreated rats. When rats were fed BRB supplements after NMBA treatment, the expression of VEGF-C mRNA was reduced approximately 50% of the level in animals treated with NMBA only in animals treated with NMBA plus BRB supplements.

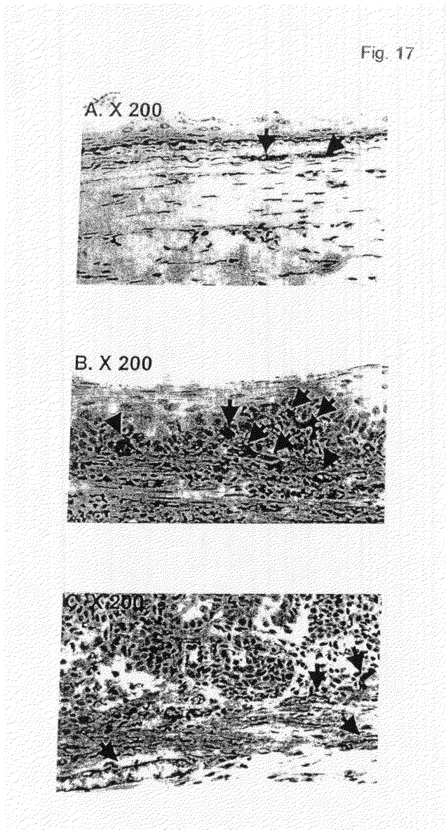

The altered VEGF-C expression resulting from the supplementation of the animal diet with the BRB composition is correlated with an alteration in microvessel differentiation. FIG. 17 shows microvessel density staining pattern in subepithelial plexus of normal epithelium (A), NMBA-treated epithelium (B) and NMBA+BRB-treated epithelium (C); While normal esophageal epithelium contained few capillaries, in rats treated with NMBA only, the esophageal epithelium microvessels were more frequent and more densely packed. In contrast, rats fed a diet supplemented with a BRB composition had comparatively fewer microvessels when challenged with NMBA. The quantification of vascularization revealed a significant reduction in MVD from 53.7±5.6 microvessels/cm in animals treated with NMBA only to 22.6±2.6 microvessels/cm in animals treated with NMBA plus BRB (P<0.0001).

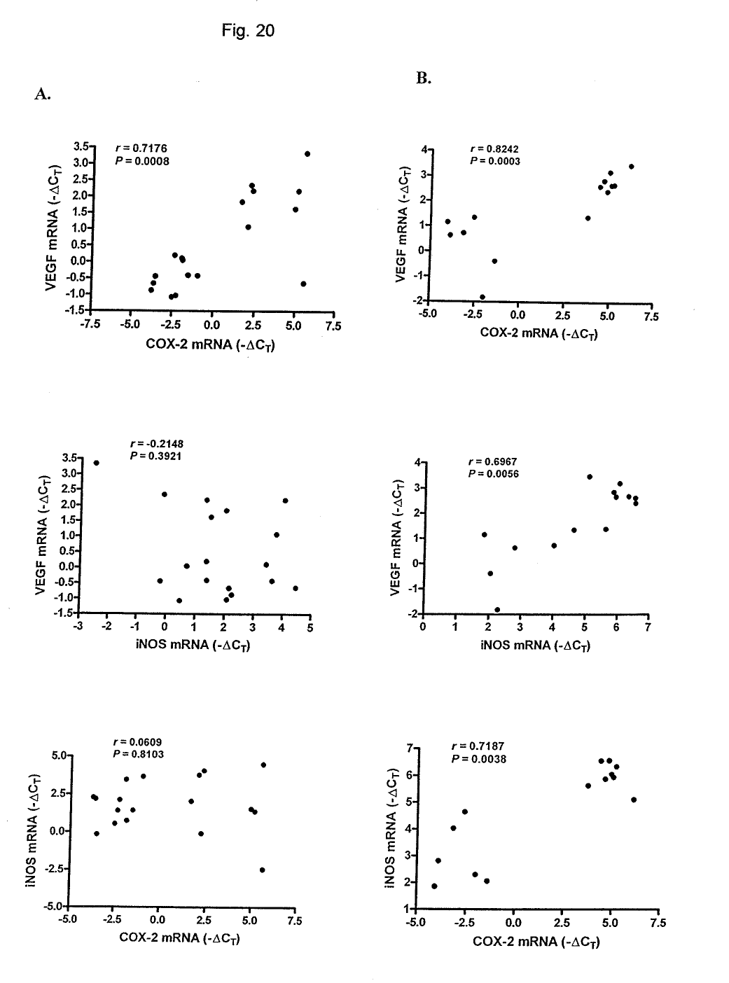

A further correlation between the expression of VEGF, COX-2 and iNOS occurs during NMBA-induced esophageal tumorigenesis. To better elucidate the pleiotropic effects of the supplementation of the animal diet with BRB composition, the correlation between down-regulation the mRNA expression of VEGF, COX-2 and iNOS was analyzed using Spearman's rank correlation coefficient. As shown in FIG. 20 (panel A), in rats treated with NMBA only, the expression of VEGF was correlated with the expression of COX-2 (r²=0.72, P<0.001) but not with the expression of iNOS. In rats treated with NMBA+BRB (FIG. 20 panel B), however, the expression of VEGF was correlated with both the expression of COX-2 (r²=0.86, P<0.001) and iNOS (r²=0.81, P<0.005). Moreover, the expression of COX-2 and iNOS is also correlated (r²=0.72, P<0.005).

### Example 11

### Inhibitory Effects of Black Raspberry Composition on Inducible Nitric Oxide Synthase and Cyclooxygenase-2 in Esophageal Carcinogenesis

The effect of PBIT treatment on increased expression of the inducible isoform of nitric oxide synthase (iNOS) and of cyclooxygenase-2 (COX-2) in the development of NMBA-induced tumors in the rat esophagus led us to postulate that the inhibition of NMBA-induced tumors in the rat esophagus by BRB may also have been associated with down-regulation of iNOS and COX-2. In order to test an inhibitory effect of BRB extract and BRB extract fraction, we conducted a study to determine if dietary freeze-dried BRB extract inhibits tumor development and progression by down-regulating iNOS and COX-2 activities. The results of this study demonstrates that modulation of these two enzymes by BRB is associated with inhibition of esophageal carcinogenesis in the rat.

Experimental Protocol:

Following a two-week acclimation period to the animal facility, 150 rats were randomized into four experimental groups and placed on AIN-76A diet. Rats were treated with NMBA (0.25 mg/kg b.w.) three times per week for 5 weeks. Black raspberries were administered at 5% of the diet following NMBA treatment and for the duration of the bioassay. Rats in Group 1 were injected s.c. with 0.2 ml of a solution of 20% DMSO in water, the solvent for NMBA, three times per week for five weeks. Animals in Group 2 were fed AIN-76A diet containing 5% BRB for the duration of the bioassay. Rats in Groups 3 and 4 were injected s.c. with 0.2 ml of NMBA (0.25 mg/kg body weight) in 20% DMSO:H₂O three times per week for five weeks. Three days following the final NMBA treatment, all rats in Groups 3 and 4 were given AIN-76A diet containing 5% BRB for the duration of the bioassay. Food consumption and body weight data were recorded weekly. At 9 and 15 weeks, 5 rats from Groups 1 & 2 and 10 rats from Groups 3 & 4; and, at 25 weeks, 15 rats from Groups 1 & 2 and 30 rats from Groups 3 & 4, were euthanized by CO₂ asphyxiation and subjected to gross necropsy. The esophagus of each rat was excised and opened longitudinally. Tumors larger than 0.5 mm in a single dimension were counted, mapped, and measured by length, width and height. Tumor volume was calculated using the formula for a prolate spheroid: length×width×height×π/6. After the tumor data were recorded, the esophagus was cut longitudinally into two parts. Tumors were removed from the esophagus and frozen in liquid nitrogen. The epithelium was stripped of the submucosal and muscularis layers and frozen in liquid nitrogen separately. All samples were stored at −80° C. until analysis.

TABLE 9
Experimental design for bioassay with black raspberries (BRB)

| | | | | Dose Admin. | |
|---|---|---|---|---|---|
| Gr. | Treatment | # of Rats | Volume (ml) | (mg/kg b.w.) | Diet |
| 1 | DMSO + | 25 | 0.2 | 0 | Control |
| | H₂O^{a} | | | | AIN- 76A |
| 2 | None | 25 | 0 | 0 | AIN-76A + |
| | | | | | 5% BRB |
| 3 | NMBA | 50 | 0.2 | 0.25 | Control |
| | | | | | AIN-76A |
| 4 | NMBA | 50 | 0.2 | 0.25 | AIN-76A + |
| | | | | | 5% BRB |

^{a}DMSO + H₂O, vehicle for NMBA.
^{b} 0.25 mg/kg body weight NMBA injected s.c. three times per week for 5 weeks.

General Observations.

The mean body weights and food consumption in rats (Groups 1-4) treated with either NMBA or 20% DMSO in water and fed either control or berry containing diets were not significantly different throughout the bioassay. There were no observable gross or histopathological changes in the lungs, liver, kidneys, small intestine and colon of rats treated with BRB only. All tumor specimens removed from the esophagus at necropsy were found to be papillomas by histopathological examination. At weeks 9 and 15 of the bioassay, freeze-dried BRB did not exhibit a significant effect on tumor development. By week 25, none of the DMSO treated rats (Group 1) or the rats fed 5% BRB alone (Group 2) had developed esophageal tumors (Table 10). For those groups treated with NMBA, dietary BRB extract added to the animal diet reduced the incidence of esophageal tumors from 96% in rats treated with NMBA only (Group 3) to 89% in rats treated with NMBA+5% BRB (Group 4). Tumor volume was reduced from an average of 5.68±0.86 mm³ in NMBA treated rats to an average of 4.75±1.11 mm³ in NMBA treated rats given a diet supplemented with BRB extract. While, the reductions in tumor incidence and volume did not reach the significance of (P<0.05), in contrast, addition of dietary BRB extract significantly reduced tumor multiplicity from 3.78±0.41 tumors per esophagus in NMBA treated rats to 2.23±0.21 tumors per esophagus in NMBA treated rats given a diet supplemented with BRB extract (P<0.001).

TABLE 10
Effect of black raspberries (BRB) on post-initiation events of
NMBA-induced tumorigenesis in the rat esophagus at 25 weeks

| | | | | Tumor | Tumor | Tumor Volume |
|---|---|---|---|---|---|---|
| | | | No. of | Incidence | Multiplicity | (mm³)^{a} |
| Group | NMBA | Diet | Rats | (%) | Mean ± S.E. | Mean ± S.E. |
| 1 | − | Control | 10 | 0 | 0 | 0 |
| | | AIN-76A | | | | |
| 2 | − | AIN-76A + | 15 | 0 | 0 | 0 |
| | | 5% BRB | | | | |
| 3 | + | Control | 28 | 96.4 | 3.78 ± 0.41 | 5.68 ± 0.86 |
| | | AIN-76A | | | | |
| 4 | + | AIN-76A + | 29 | 89.66 | 2.23 ± 0.21^{b} | 4.75 ± 1.11 |
| | | 5% BRB | | | | |

^{a}Tumor volume calculated as length × width × depth × π/6 assuming a prolate spheroid shape.
^{b}Significantly lower than Group 3 as determined by analysis of variance (P < 0.001).

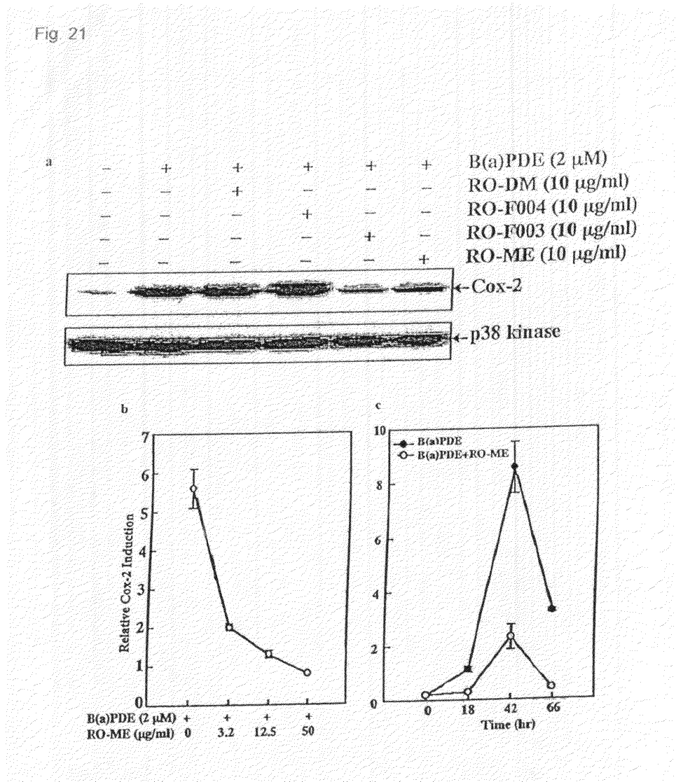

The reduction in tumor multiplicity is associated with a reduction in iNOS and COX-2 mRNA expression. To determine whether the inhibition of tumor development in the rat esophagus by BRB is associated with modulation of either iNOS and/or COX-2 mRNA expression, Real-Time RT-PCR was performed on samples of esophageal epithelium and papillomas collected from NMBA-treated rats fed either the control or the 5% BRB diets. Histopathologically, esophagi collected from solvent-treated rats were classified as normal, and those from NMBA-treated rats after the removal of papillomas were classified as preneoplastic (containing areas of hyperplasia and dysplasia). Papillomas were defined as exophytic lesions larger than 0.5 mm in a single dimension, and they were removed from the esophagus and stored separately. As shown in FIG. 20, BRB suppressed the expression of iNOS mRNA in preneoplastic lesions by 95% (P<0.01) and in papillomas by 60% (P<0.05). The effects of BRB on COX-2 mRNA expression is shown in FIG. 21. BRB reduced the expression of COX-2 mRNA in preneoplastic tissues by 60% (P<0.01) but did not reduce the expression levels in papillomas.

Freeze-dried BRB composition also affected iNOS and COX-2 activities in esophageal tissues, as shown by measuring total nitrate and nitrite levels and prostaglandin E₂ (PGE₂) levels, respectively, using an enzyme immunoassay. BRB decreased total nitrate and nitrite levels from 4.40±0.52 to 4.17±0.83 μM(not significant) in preneoplastic lesions, and from 7.66±1.65 to 4.04±1.24 μM in papillomas (47% reduction; P<0.05) (Table 11). Similar inhibitory effects on COX-2 activity were observed. PGE₂ levels were reduced in preneoplastic lesions from 3.05±0.59 μg/mg protein in rats treated with NMBA only to 1.22±0.13 μg/mg protein in rats treated with NMBA+BRB (60% reduction; P<0.01). In addition, BRB treatment suppressed PGE₂ production in papillomas from 18.10±4.40 μg/mg protein in rats treated with NMBA only to 6.98±1.68 μg/mg protein in rats treated with NMBA+BRB (61% reduction; P <0.05).

TABLE 11
Modulation of iNOS and COX-2 activities in esophagus
by black raspberries (BRB) in NMBA-treated rats

| | Preneoplasia | | Papilloma | |
|---|---|---|---|---|
| Diet | iNOS^{a} | COX-2^{b} | iNOS | COX-2 |
| Control | 4.40 ± 0.52^{c} | 3.05 ± 0.59 | 7.66 ± 1.65 | 18.10 ± 4.40 |
| AIN-76A | | | | |
| AIN-76A + | 4.17 ± 0.83 | 1.22 ± 0.13^{e} | 4.04 ± 1.24^{d} | 6.98 ± 1.68^{d} |
| 5% BRB | | | | |

^{a}iNOS activity is expressed as μM.
^{b}COX-2 activity is expressed as μg/mg protein.
^{c}Mean ± SE.
^{d}Significantly lower than rats fed control diets as determined by analysis of variance (P < 0.05).
^{e}Significantly lower than rats fed control diets as determined by analysis of variance (P < 0.01).

While the invention has been described with reference to preferred embodiments, those skilled in the art will understand that various changes may be made and equivalents may be substituted for elements thereof without departing from the scope of the invention. In addition, many modifications may be made to adapt a particular situation or material to the teachings of the invention without departing from the essential scope thereof. Since certain changes may be made in the above compositions and methods without departing from the scope of the invention herein involved, it is intended that all matter contained in the above descriptions and examples or shown in the accompanying drawings shall be interpreted as illustrative and not in a limiting sense. In this application all units are in the metric system and all amounts and percentages are by weight, unless otherwise expressly indicated. All terms not specifically defined herein are considered to be defined according to Dorland's Illustrated Medical Dictionary, 27th edition, or if not defined in Dorland's dictionary then in Webster's New Twentieth Century Dictionary Unabridged, Second Edition. The disclosures of all of the citations, including patents and patent applications provided are being expressly incorporated herein by reference. The disclosed invention advances the state of the art and its many advantages include those described and claimed.

1. An isolated organic solvent fruit extract fraction having a therapeutically effective amount of activity in modulating undesired signal transduction activity useful for reducing the frequency, duration or severity of a neoplastic disease or condition in a subject, said fruit extract being derived from a plant of one or more of the genera Fragaria or Rubus.

2. A fruit extract fraction of claim 1, suitable for modulating the activity of one or more of the molecule NF-Kβ, the molecule AP-1, the molecule Akt, the molecule iNOS, and the molecule VEGF

3. The fruit extract fraction of claim 1, wherein said disease or condition is selected from the group consisting of a malignancy, a neoplasia, a cardiovascular disease, a thrombotic disease, an atherogenic disease, an inflammatory disease or condition, an immunological disease, a neurological disease, a dermatological disease, an opthalmological disease, or aging.

4. The fruit extract fraction of claim 1, wherein said disease or condition treated is selected from the group consisting of a malignancy, cancers of the aerodigestive tract in animals, oral cancer, laryngeal cancer, pharyngeal cancer, esophageal cancer, esophageal squamous cell carcinoma, esophageal adenocarcinoma, stomach cancer, colon cancer, epithelial dysplasia of the esophagus, development of Barrett's esophagus, oral leukoplakia, erythroplakia, and colonic polyps.

5. The fruit extract fraction of claim 3, wherein the neoplasia is an aerodigestive tract cancer.

6. The fruit extract fraction of claim 5, wherein said aerodigestive tract cancer is oral, esophageal, or colon cancer.

7. The fruit extract fraction of claim 1, wherein said fruit extract fraction is derived from a plant of the genus Rubus.

8. The fruit extract fraction of claim 1, wherein the fruit is one or more of strawberry, raspberry, red raspberry, black raspberry, ligonberry, cloudberry, blackberry and blackberry.

9. The fruit extract fraction of claim 8, wherein the fruit is a black raspberry.

10. The fruit extract fraction of claim 1, wherein said amount of activity useful for modulating undesired signal transduction activity is present in an amount at least about 100% greater than present in a native fruit.

11. The fruit extract fraction of claim 1, wherein the fraction has antioxidant activity with an oxygen radical absorbance capacity value per milligram of fraction of one or more of at least about 5.0, 10.0, 15.0, and 20.0.

12. The fruit extract fraction of claim 1, further comprising one or more of a carotenoid, a phenolic compound, a phytosterol, a mineral, and an antioxidant.

13. The fruit extract fraction of claim 12, wherein said phenolic compound is one or more of an ellagic acid, a ferulic acid, an anthocyanidin, an anthocyanin, a cyanidin, a pelargonidin, a quercetin, a kaempferol, and analogs thereof.

14. The fruit extract fraction of claim 13, wherein said phenolic compound is an anthocyanin.

15. A foodstuff comprising a food fortified with a fruit extract fraction according to claim 1, and capable of delivering a chemopreventative agent to the gastrointestinal tract of a patient.

16. A dietary supplement comprising a consumable supplement fortified with a fruit extract fraction according to claim 1, capable of delivering a chemopreventative agent to the gastrointestinal tract, and with less than one half the free sugar of the native fruit.

17. A pharmaceutical composition comprising a fraction according to claim 1, or one or more substitutents of said fraction derived therefrom, and a pharmaceutically acceptable carrier therefor.

18. A method for treating or preventing a disease or condition in a subject comprising the step of administering to said subject a therapeutically-effective amount of a foodstuff, dietary supplement or pharmaceutical composition fortified with an organic solvent fruit extract fraction having a therapeutically effective amount of activity in modulating undesired signal transduction activity useful for reducing the frequency, duration or severity of a disease or condition in a subject, said fruit extract being derived from a plant of one or more of the genera Fragaria or Rubus.

19. The method of claim 18, wherein said disease or condition is selected from the group consisting of a malignancy, a cardiovascular disease, a thrombotic disease, an atherogenic disease, an inflammatory disease or condition, an immunological disease, a neurological disease, a dermatological disease, an opthalmological disease, or aging.

20. The method of claim 19, wherein the malignancy is an aerodigestive tract cancer.

21. The method of claim 20, wherein said aerodigestive tract cancer is oral, esophageal, or colon cancer.

22. The method of claim 21, wherein said subject has, or is at risk for acquiring, a malignancy.

23. A fruit extract fraction product prepared according to process comprising,
a) harvesting fruit from a plant of one or more of the genera Fragaria or Rubus and chilling said fruit to about 4° C. within four hours;
b) physically disrupting an amount of chilled fruit;
b) maintaining the disrupted fruit at a low temperature of less than about 4° C. until fractionated;
c) removing an amount of water content from the disrupted fruit by sublimation under a vacuum of less than about 400 millitorr;
d) adding to the fruit extract an organic solvent to produce an extract/solvent mixture; and
e) removing the solvent portion of the extract/solvent mixture thereby producing isolated fruit extract fraction substantially free of solvent,
wherein the activity of the fruit extract fraction is has at least about a three fold increase in anti-dysplastic activity compared to the undisrupted fruit as measured by the activity of the fruit extract fraction in inhibiting one or more of AP-1, NFKB, Akt, COX-2, and VEGF.

24. The product of claim 23, wherein the fruit is selected from one or more of strawberry, raspberry, red raspberry, black raspberry, ligonberry, cloudberry, blackberry and combinations thereof.

25. The product of claim 24, wherein the fruit is black raspberry.

26. The product of claim 23, wherein said vacuum is at least about 200 millitorr.

27. The product of claim 23, wherein the low temperature is less than about −20° C.

28. The product of claim 23, wherein the organic solvent is selected from the group consisting of dichloromethane, methanol, ethanol, acetone, and combinations thereof.

29. The product of claim 28, wherein the organic solvent is about a 1:1 combination of dichloromethane and methanol.

30. The product of claim 28, wherein the organic solvent is about a 1:1 combination of acetone and methanol.

31. The product of claim 28, wherein the organic solvent is about an 80:20 combination of ethanol and water.

32. The product of claim 23, wherein said product is useful for reducing the frequency, duration or severity of a disease or condition in a subject disease or condition in a subject and said disease or condition is selected from the group consisting of a malignancy, a neoplasia, a cardiovascular disease, a thrombotic disease, an atherogenic disease, an inflammatory disease or condition, an immunological disease, a neurological disease, a dermatological disease, an opthalmological disease, or aging.

33. The isolated fruit extract fraction of claim 23, in a form suitable for use in one or more of a foodstuff, a dietary supplement, and a pharmaceutical composition.

34. A method of producing a fruit extract fraction comprising,
a) harvesting fruit and chilling to about 4° C. within four hours;
b) physically disrupting an amount of chilled fruit;
b) maintaining the disrupted fruit at a low temperature of less than about 4° C. until fractionated;
c) removing an amount of water content from the disrupted fruit by sublimation under a vacuum of less than about 200 millitorr;
d) adding to the fruit extract an organic solvent to produce an extract/solvent mixture; and
e) removing the solvent portion of the extract/solvent mixture thereby producing isolated fruit extract fraction substantially free of solvent,
wherein the activity of the fruit extract fraction is has at least about a five fold increase in anti-dysplastic activity compared to the undisrupted fruit as measured by the activity of the fruit extract fraction in inhibiting one or more of AP-1, NFKB, Akt, COX-2, and VEGF.

35. The product of claim 34, wherein the fruit is selected from one or more of strawberry, raspberry, red raspberry, black raspberry, ligonberry, cloudberry, blackberry and combinations thereof.

36. The product of claim 34, wherein the organic solvent is selected from the group consisting of dichloromethane, methanol, ethanol, acetone, and combinations thereof.

37. The method of claim 36, wherein the organic solvent is about an 80:20 combination of ethanol and water.
